(19) United States
(12) Patent Application Publication
(10) Pub. No.: US 2024/0043394 A1
(43) Pub. Date: Feb. 8, 2024

(54) PROTEASOME ENHANCERS AND USES THEREOF

(71) Applicant: Board of Trustees of Michigan State University, East Lansing, MI (US)

(72) Inventors: Jetze J. Tepe, East Lansing, MI (US); Sophia Staerz, Lansing, MI (US)

(21) Appl. No.: 18/041,193

(22) PCT Filed: Aug. 10, 2021
(86) PCT No.: PCT/US2021/045448

§ 371 Date: Feb. 9, 2023

Related U.S. Application Data
(60) Provisional application No. 63/064,262, filed on Aug. 11, 2020.

(51) Int. Cl. C07D 279/28; C07D 265/38; C07D 265/36; C07D 209/88; C07D 471/04; C07D 473/40; C07D 233/64; C07D 235/26; C07D 207/327; C07D 295/192

(52) U.S. Cl. CPC C07D 279/28; C07D 265/38; C07D 265/36; C07D 209/88; C07D 471/04; C07D 473/40; C07D 233/64; C07D 235/26; C07D 207/327; C07D 295/192

(57) ABSTRACT

Described herein are chlorpromazine, methods for making such compounds, and the use of such compounds in the treatment of cancer, an inflammatory disease or condition or neurodegenerative diseases, such as Parkinson's disease, Alzheimer's disease, Huntington's disease, and ALS.

### CROSS-REFERENCE TO RELATED APPLICATIONS

This application claims the benefit of U.S. Provisional Appl. Ser. No. 63/064,262, filed Aug. 11, 2020, the entirety of which is incorporated by reference as if fully set forth herein.

### STATEMENT OF GOVERNMENT SUPPORT

This invention was made with government support under NS111347, AG061306 and GM092715 awarded by the National Institutes of Health. The government has certain rights in the invention.

### BACKGROUND

The regulation of protein synthesis, degradation, folding, trafficking and aggregation within a cell are collectively known as proteostasis. Proteostasis is maintained by a wide array of cellular machinery that work to ensure that proteins are present in the proper location, amounts and form to perform their respective functions. When one of the pathways involved with proteostasis becomes dysregulated there can be disastrous effects on the cell and even on neighboring cells. One increasingly prevalent example of this is seen in neurodegenerative diseases, such as Parkinson's disease (PD), Alzheimer's disease (AD), Huntington's disease (HD) and amyotrophic lateral sclerosis (ALS). In these neurodegenerative diseases, accumulation of specific aggregation-prone proteins (hereafter referred to as intrinsically disordered proteins (IDPs)) leads to toxic signaling and disruption of proteostasis caused by their uncontrolled aggregation and oligomerization (hereafter, aggregation and oligomerization are used interchangeably). For example, the IDP α-synuclein (α-syn) and its oligomers are associated with the pathogenesis of PD. IDPs are named for their lack of tertiary structure allowing them to adopt numerous conformations and interact with multiple binding partners. IDPs are generally short-lived signaling proteins or transcription factors that are highly bound to other cellular components keeping free cytosolic levels low. Additionally, unbound IDPs are readily degraded by the 20S proteasome, the default protease responsible for IDP digestion. The accumulation of IDPs seen in neurodegenerative diseases can begin as a result of one of several disruptions (e.g. mutations, changes in expression, oxidative stress, aging, proteasome impairment, etc.) to their normal regulation. While α-syn may not be the sole cause of PD, there is strong evidence supporting its key role in the disease, including familial forms of PD resulting from mutations in the SNCA gene. Elevated monomeric α-syn levels are also known to cause apoptosis-inducing aggregation in neurons. Additionally, oligomeric forms of α-syn and other IDPs have recently been shown to directly inhibit the proteasome, further disrupting its ability to regulate IDPs concentrations. These data collectively suggest that the accumulation of α-syn and formation of oligomeric species of the IDP play a critical role in the progression of PD. Due to a lack of defined binding pockets, IDPs such as α-syn, and their aggregation are difficult to target through traditional small molecule drug design. There are currently no effective treatments to hinder the progression of neurodegenerative diseases that are associated with IDP accumulation.

### BRIEF DESCRIPTION OF THE FIGURES

The drawings illustrate generally, by way of example, but not by way of limitation, various embodiments discussed herein.

FIG. 1 is plots of fold increase in proteolytic degradation of an equimolar mixture of three fluorogenic peptide probes (Suc-LLVY-AMC, Z-LLE-AMC and Boc-LRR-AMC) treated 20S or 26S proteasome in the presence of a concentration range (0-80 μM) of either CPZ, compound 2, or compound 19, compared to vehicle control. This data was collected in triplicate (n=3).

FIGS. 2A-2F are cartoons of preferred docking site of CPZ analogs, utilizing Autodock Vina and PyMol, in the α1-2 intersubunit pocket of the 20S proteasome (FIG. 2A); a cartoon of the location of two amino acid residues of interest, Y159 and C161 (FIG. 2B); and cartoons of predicted binding interaction of compound 2 (FIG. 2C), compound 7 (FIG. 2D), compound 9 (FIG. 2E), and compound 19 (FIG. 2F), viewed using BIOVIA Discovery Studio 2020.

FIG. 3 shows degradation of α-synuclein by purified 20S proteasome treated with 0.1 mM, 0.5 mM, 1 mM, and 5 mM of compound 2, 8, or 19 with the previously reported TCH-165 as a positive control. Quantification of α-synuclein degradation by each compound was done in triplicate. One-way ANOVA statistical analysis was used to determine statistical significance (ns=not significant, *p<0.05, *p<0.01, ***p<0.001, ****p<0.0001).

FIGS. 4A-4B show representative Western blot (1 of 4) of A53T α-synuclein in Hek-293T cells treated with vehicle (DMSO), BTZ, TCH-165, compound 19, compound 2 or inactive control 8 (FIG. 4A); and quantitation of A53T α-syn remaining (using A53T α-syn/β-actin ratio of 4 separate experiments) for treatments normalized to vehicle control (100%). Error bars denote standard deviation. One-way ANOVA statistical analysis was used to determine statistical significance (ns=not significant, *p<0.05, **p<0.01, ***p<0.001, ****p<0.0001) (FIG. 4B).

### SUMMARY

The disclosure relates to small molecules that enhance proteasome function and restore the activity of impaired proteasomes. Small molecule proteasome enhancers prevent the toxic accumulation of aggregation-prone proteins and prevent neuronal cell death caused by aggregation-prone proteins. The disclosure therefore relates to the use of small molecules as therapeutic agents to treat neurodegenerative diseases. Neurodegenerative diseases include, but are not limited to Alzheimer's disease (AD) and other dementias, Parkinson's disease (PD) and PD-related disorders, Prion disease, Motor neuron diseases (MND), Huntington's disease (HD), Spinocerebellar ataxia (SCA), Spinal muscular atrophy (SMA).

### DESCRIPTION

Currently, there are no available therapeutics to prevent or slow down the progression of neurodegenerative diseases, such as Alzheimer's and Parkinson's. Currently, there are no available therapeutics to prevent or slow down the progression of neurodegenerative diseases, such as Alzheimer's and Parkinson's. The disclosure relates to a chemotype that has been shown herein to be a biologically active enhancers of mammalian proteasomes. The chemotype described herein is based on chlorpromazine and derivatives thereof.

The disclosure relates to chlorpromazine and derivates thereof, and their use to, among other things, prevent or slow down the progression of neurodegenerative diseases. The disclosure therefore relates to compounds of the formula (I):

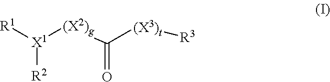

or a pharmaceutically acceptable salt, polymorph, prodrug, solvate or clathrate thereof, wherein:
R¹ is hydrogen, alkyl, cycloalkyl, aryl, heterocyclyl or heteroaryl;
R² is hydrogen, alkyl, cycloalkyl, aryl, heterocyclyl or heteroaryl;
R³ is aryl or heteroaryl (e.g., tricyclic heteroaryl, such as phenothiazine and carbazole; and bicyclic heteroaryl, such as indole);
X¹ is N or CR⁵, wherein R⁵ is absent (e.g., when X¹ is alkenyl), hydrogen, alkyl, heterocyclyl or aryl;
X² is alkyl or alkenyl;
X³ is alkyl or alkenyl;
g is 0 or 1; and
t is 0 or 1;
provided that g and t are not simultaneously 0. In some examples, g is 0 and t is 1. In other examples, g is 1 and t is 0.

One example of a compound of the formula (I) is a compound of the formula (Ia):

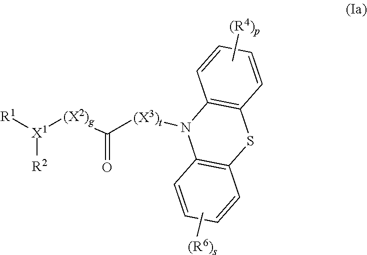

or a pharmaceutically acceptable salt, polymorph, prodrug, solvate or clathrate thereof, wherein:
R¹ is hydrogen, alkyl, cycloalkyl, aryl, heterocyclyl or heteroaryl;
R² is hydrogen, alkyl, cycloalkyl, aryl, heterocyclyl or heteroaryl;
R³ is aryl or heteroaryl (e.g., tricyclic heteroaryl, such as phenothiazine and carbazole; and bicyclic heteroaryl, such as indole);
X¹ is N or CR⁵, wherein R⁵ is absent (e.g., when X¹ is alkenyl), hydrogen, alkyl, heterocyclyl or aryl;
X² is alkyl or alkenyl;
X³ is alkyl or alkenyl;
g is 0 or 1; and
t is 0 or 1;
provided that g and t are not simultaneously 0;
R⁴ is alkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R⁶ is alkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
p is 0, 1, 2, 3 or 4; and
s is 0, 1, 2, 3 or 4.

Yet another example of a compound of formula (I) are compounds of the formulae (Ib) and (Ic):

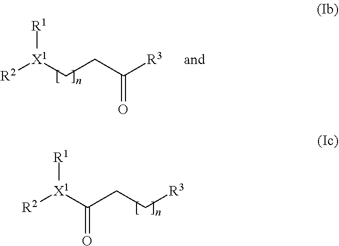

or a pharmaceutically acceptable salt, polymorph, prodrug, solvate or clathrate thereof,
wherein:
n is 0, 1 or 2.

Yet another example of a compound of formula (I) are compounds of the formulae (Id) and (Ie):

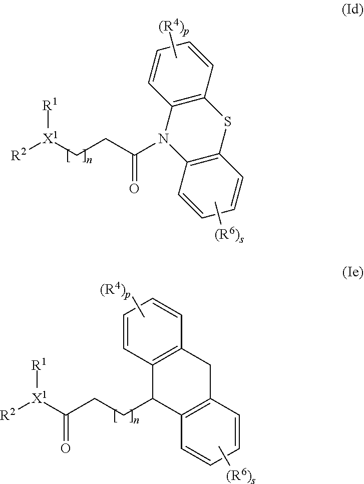

or a pharmaceutically acceptable salt, polymorph, prodrug, solvate or clathrate thereof,
wherein:
n is 0, 1 or 2.

Yet another example of a compound of formula (I) is a compound of the formula (If):

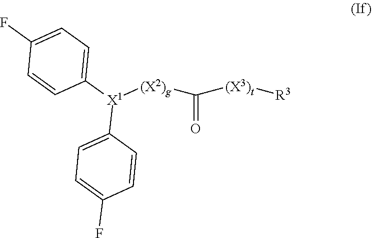

or a pharmaceutically acceptable salt, polymorph, prodrug, solvate or clathrate thereof, wherein:
R³ is aryl or heteroaryl (e.g., tricyclic heteroaryl, such as phenothiazine and carbazole; and bicyclic heteroaryl, such as indole);
X¹ is N or CR⁵, wherein R⁵ is absent (e.g., when X¹ is alkenyl), hydrogen, alkyl, heterocyclyl or aryl;
X² is alkyl or alkenyl;
X³ is alkyl or alkenyl;
g is 0 or 1; and
t is 0 or 1;
provided that g and t are not simultaneously 0. In some examples, g is 0 and t is 1. In other examples, g is 1 and t is 0.

One example of a compound of the formula (I) is a compound of the formula (Ig):

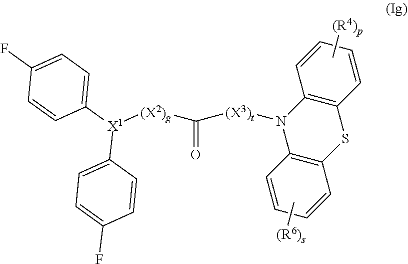

or a pharmaceutically acceptable salt, polymorph, prodrug, solvate or clathrate thereof, wherein:
X¹ is N or CR⁵, wherein R⁵ is absent (e.g., when X¹ is alkenyl), hydrogen, alkyl, heterocyclyl or aryl;
X² is alkyl or alkenyl;
X³ is alkyl or alkenyl;
g is 0 or 1; and
t is 0 or 1;
provided that g and t are not simultaneously 0. In some examples, g is 0 and t is 1. In other examples, g is 1 and t is 0.

Yet another example of a compound of formula (I) are compounds of the formulae (Ih) and (Ii):

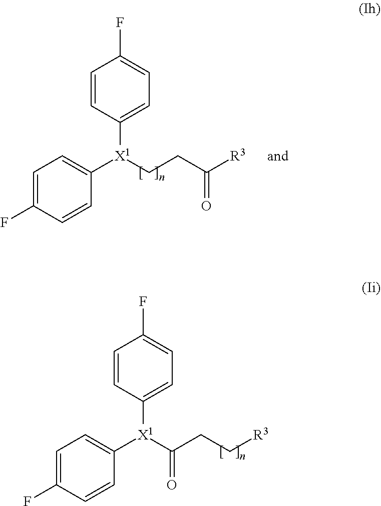

or a pharmaceutically acceptable salt, polymorph, prodrug, solvate or clathrate thereof,
wherein:
n is 0, 1 or 2.

Yet another example of a compound of formula (I) are compounds of the formulae (Ij) and (Ik):

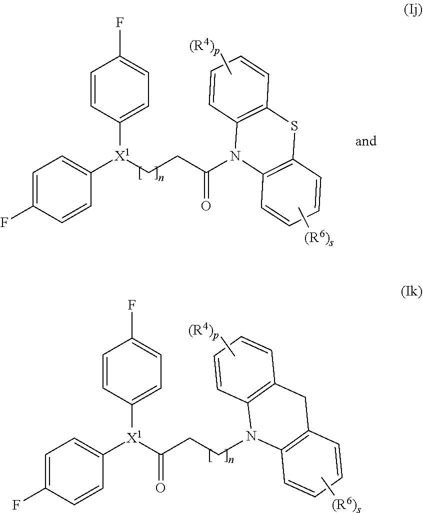

or a pharmaceutically acceptable salt, polymorph, prodrug, solvate or clathrate thereof,
wherein:
n is 0, 1 or 2.

Yet another example of a compound of formula (I) are compounds of the formula (II):

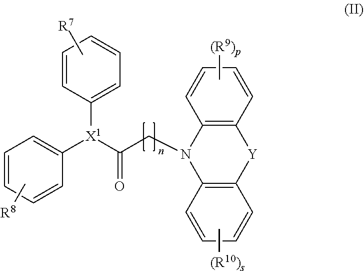

or a pharmaceutically acceptable salt, polymorph, prodrug, solvate or clathrate thereof,
wherein:
n is 0, 1 or 2;
Y is S(O)ₓ, wherein x is 0, 1 or 2, O, CH₂, or Y is N—R¹¹, wherein R¹¹ is a carbon with at least one halo (e.g., one to three halo, such as CHF₂, CCF₃, CCl₃), alkyl, aryl, acyl or heterocyclyl;
R⁷, R⁸, R⁹, and R¹⁰ are each independently halo, a carbon with at least one halo (e.g., one to three halo, such as CHF₂, CCF₃, CCl₃), alkyl, aryl, acyl or heterocyclyl;
p is 0, 1, 2, 3 or 4; and
s is 0, 1, 2, 3 or 4.

In the compounds of the formulae (I) and (Ia)-(Ik), the alkyl, cycloalkyl, aryl, heterocyclyl, and heteroaryl groups of R¹ can be unsubstituted or substituted as described herein. For example, when the alkyl, cycloalkyl, aryl, heterocyclyl, or heteroaryl groups of R¹ are substituted, they can be substituted with halo (e.g., Cl, Br, and F), amino, OR¹², wherein R¹² is hydrogen, alkyl, cycloalkyl, aryl or arylalkyl, S(O)ₓ, wherein x is 0, 1 or 2, acyl, amido or heterocyclyl.

In the compounds of the formulae (I) and (Ia)-(Ik), the alkyl, cycloalkyl, aryl, heterocyclyl, and heteroaryl groups of R² can be unsubstituted or substituted as described herein. For example, when the alkyl, cycloalkyl, aryl, heterocyclyl, or heteroaryl groups of R² are substituted, they can be substituted with halo (e.g., Cl, Br, and F), amino, OR¹², wherein R¹² is hydrogen, alkyl, cycloalkyl, aryl or arylalkyl, S(O)ₓ, wherein x is 0, 1 or 2, acyl, amido or heterocyclyl.

In the compounds of the formulae (I) and (Ia)-(Ik), the aryl or heteroaryl groups of R³ can be unsubstituted or substituted as described herein. For example, when the aryl or heteroaryl groups of R³ are substituted, they can be substituted with alkyl, cycloalkyl, aryl, heteroaryl, halo (e.g., Cl, Br, and F), amino, OR¹², wherein R¹² is hydrogen, alkyl, cycloalkyl, aryl or arylalkyl, S(O)ₓ, wherein x is 0, 1 or 2, acyl, amido or heterocyclyl.

An example of a compound of the formula (I) includes, but is not limited to, a compound of the formula:

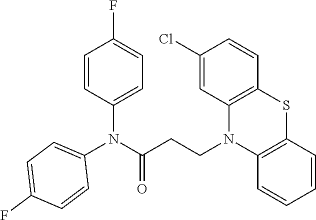 [SMILES: O=C(CCN1c2ccccc2Sc2ccc(Cl)cc21)N(c1ccc(F)cc1)c1ccc(F)cc1]

pharmaceutically acceptable salts, polymorphs, prodrugs, solvates or clathrates thereof.

The disclosure also relates to compounds of the formula (III):

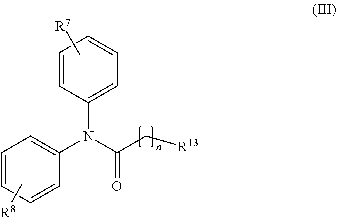

or a pharmaceutically acceptable salt, polymorph, prodrug, solvate or clathrate thereof,
wherein:
n is 0, 1 or 2;
R⁷ and R⁸ are each independently halo, a carbon with at least one halo (e.g., one to three halo, such as CHF₂, CCF₃, CCl₃), alkyl, aryl, acyl or heterocyclyl; and
R¹³ is a heterocyclyl group of the formula:

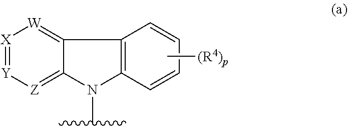

wherein W is N or C—R¹⁴; X is N or C—R¹⁴; Y is N or C—R¹⁴; and Z is N or C—R¹⁴; wherein R¹⁴ is hydrogen, an electron withdrawing group, alkyl, cycloakyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R⁴ is alkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4;

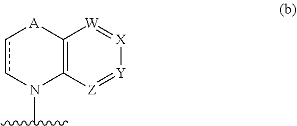

wherein the dashed line can represent a double bond;
A is S(O)ₓ, wherein x is 0, 1 or 2; O; C(R¹⁴)₂, wherein each R¹⁴ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or A is N—R¹¹, wherein R¹¹ is a carbon with at least one halo (e.g., one to three halo, such as CHF₂, CCF₃, CCl₃), alkyl, aryl, acyl or heterocyclyl;

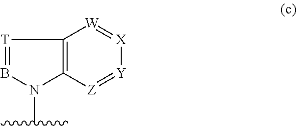

wherein T is CR¹⁴, wherein each R¹⁴ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or T is N; and
B is C—R¹⁴, wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or aryalkyl, S(O)ₓ, wherein x is 0, 1 or 2, acyl, amido or heterocyclyl; or B is N;

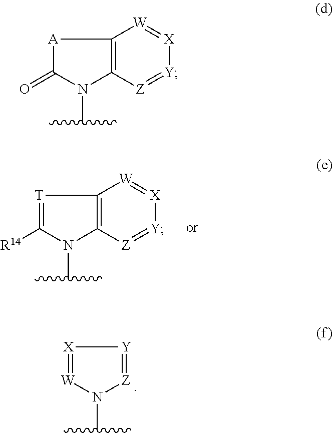

As used herein, the term “electron withdrawing group” is any atom or group that draws electron density from neighboring atoms towards itself, e.g., by resonance or inductive effects. Suitable electron withdrawing groups include, but are not limited to, halo, cyano, fluoroalkyl (e.g., CF₃), perfluoroalkyl, carboxy (CO₂H), aminocarbonyl, alkoxycarbonyl, aryloxycarbonyl, halocarbonyl (e.g., CF₃C(O)—), formyl (—C(O)H), acyl, S(O), SO₂, alkoxysulfonyl (e.g., RSO₂—), aryloxysulfonyl (e.g., aryl-O—SO₂—), perfluoroalkylsulfonyl (e.g., CF₃SO₂—), alkylsulfonyl (e.g., alkyl-SO₂—), azo (—N₃), alkenyl, alkynyl, dialkylphosphonato (e.g., (alkyl-O)₂—P(O)—), diarylphosphonato (e.g., (aryl-O)₂P(O)—), or combinations thereof.

In compounds of the formula (III), when R¹³ is a heterocyclyl of the formula (a), Y can be N. Alternatively, when R¹³ is a heterocyclyl of the formula (a), at least two of W, X, Y, and Z is N. Thus, for example, the compounds of the formula (III) can be compounds of the formulae:

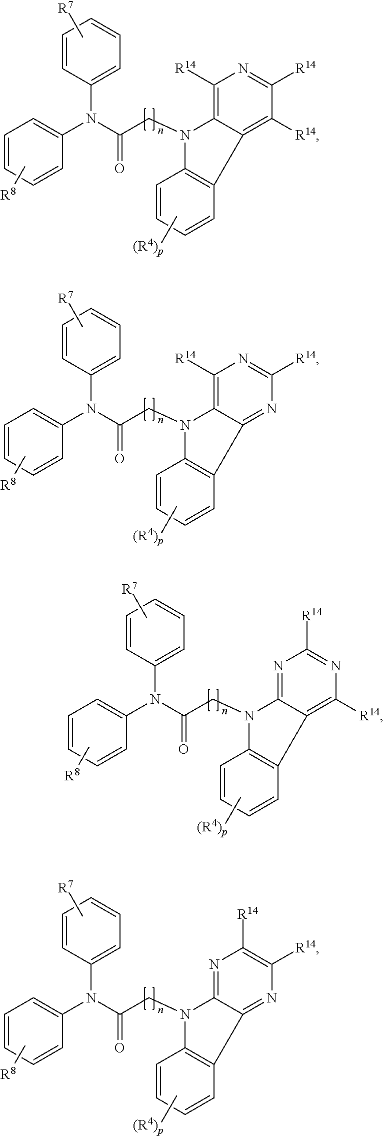

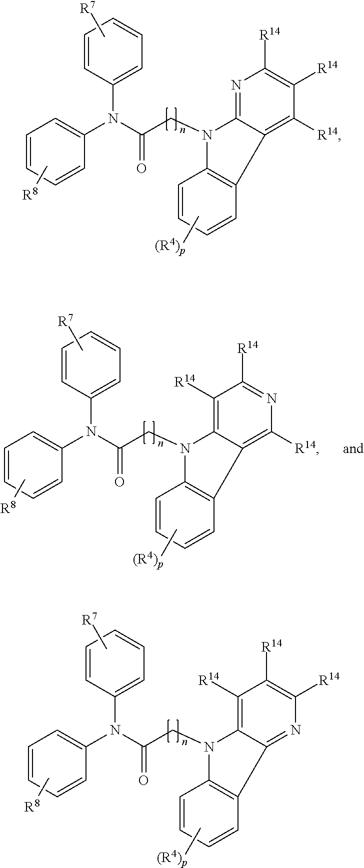

or a pharmaceutically acceptable salt, polymorph, prodrug, solvate or clathrate thereof.

In compounds of the formula (III), when R¹³ is a heterocyclyl of the formula (b), Y can be N. Alternatively, when R¹³ is a heterocyclyl of the formula (b), at least two of W, X, Y, and Z is N. Thus, for example, the compounds of the formula (III) can be compounds of the formulae:

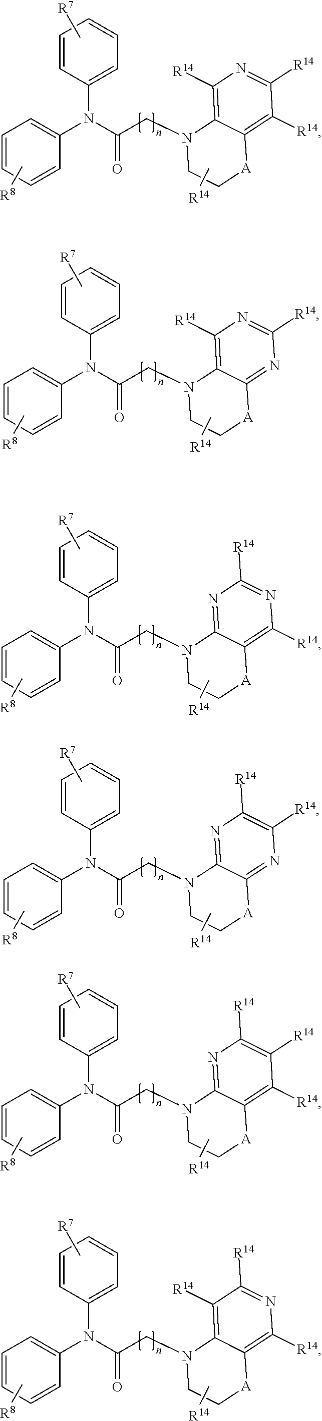

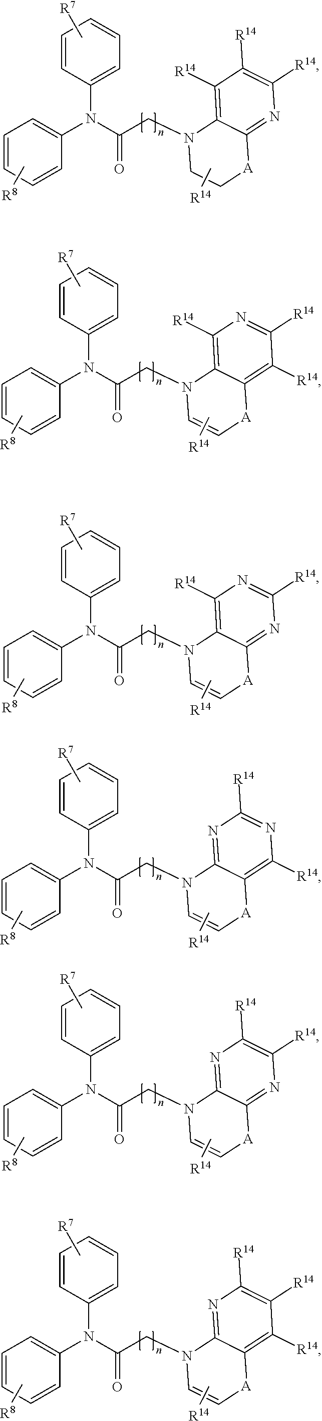

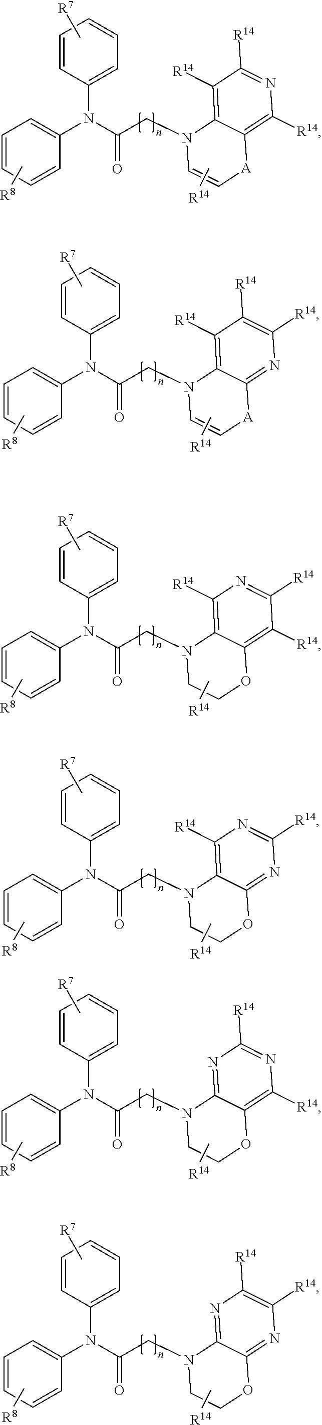

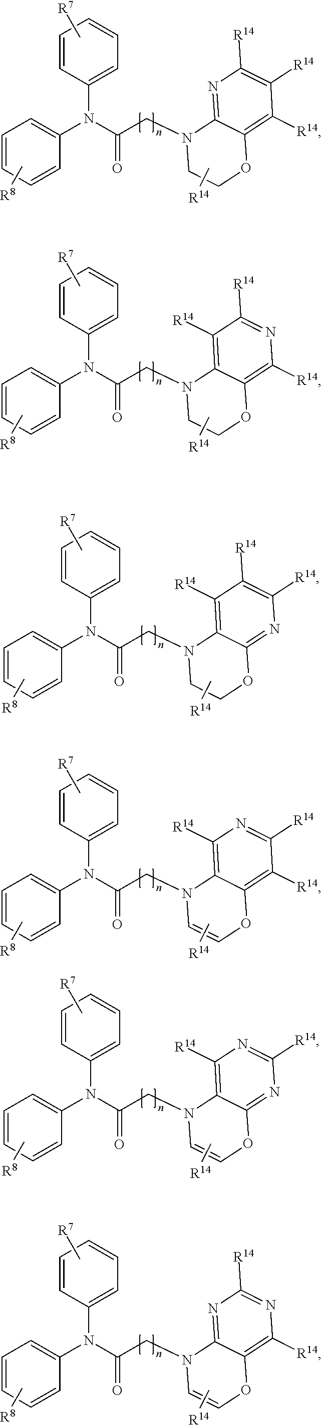

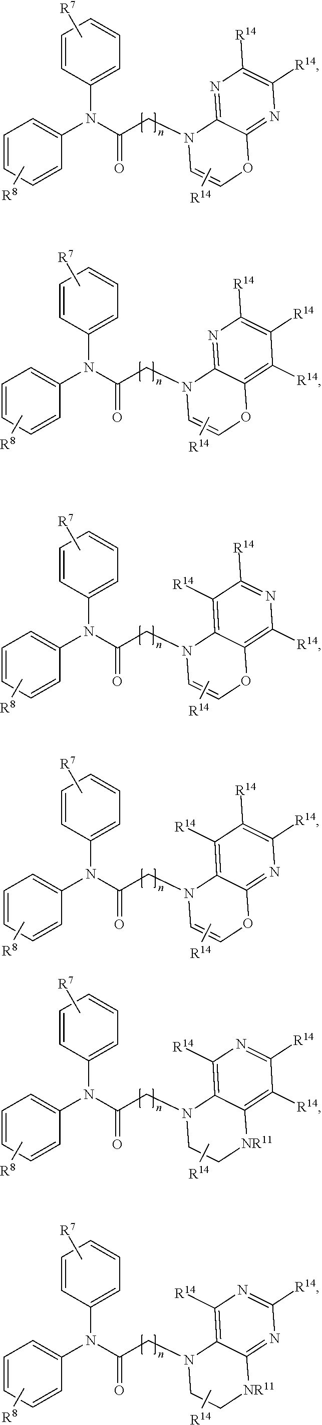

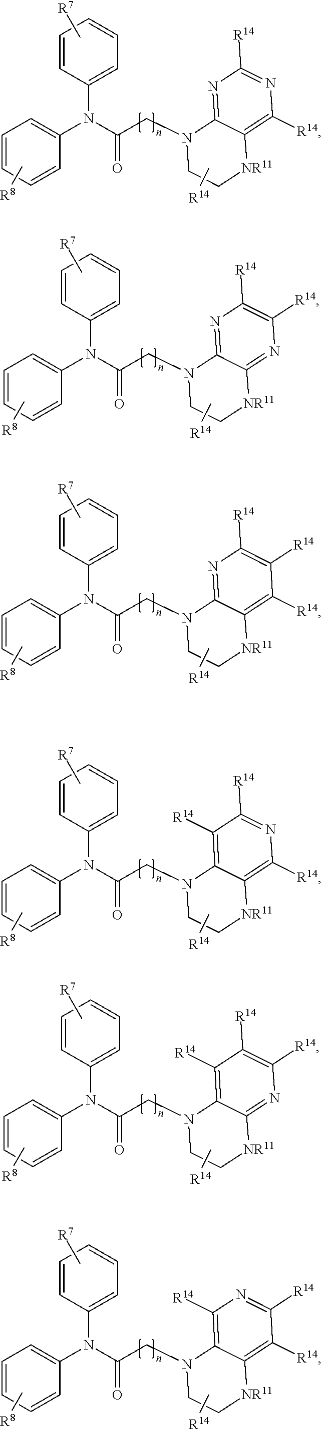

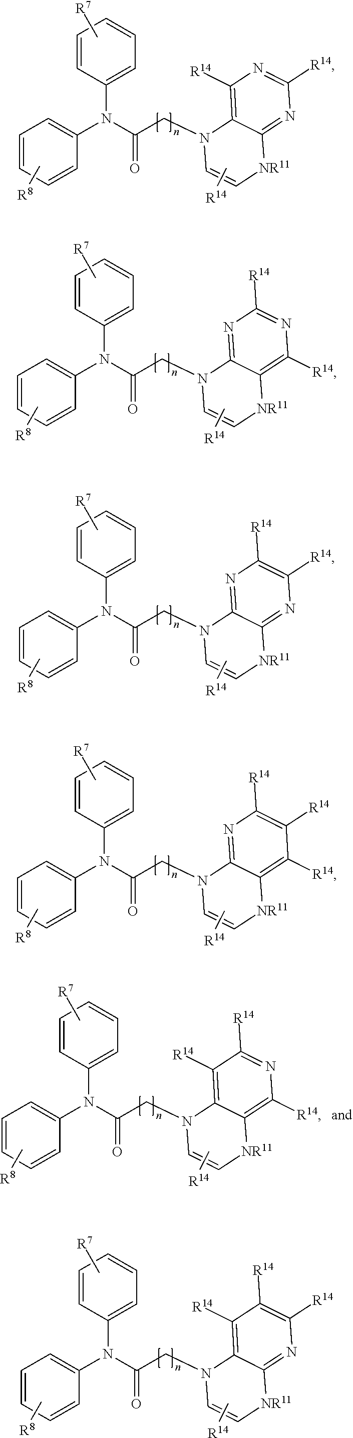

or a pharmaceutically acceptable salt, polymorph, prodrug, solvate or clathrate thereof.

In compounds of the formula (III), when R¹³ is a heterocyclyl of the formula (c) or (e), Y can be N. Alternatively, when R¹³ is a heterocyclyl of the formula (c) or (e), at least two of W, X, Y, and Z is N. Thus, for example, the compounds of the formula (III) can be compounds of the formulae:

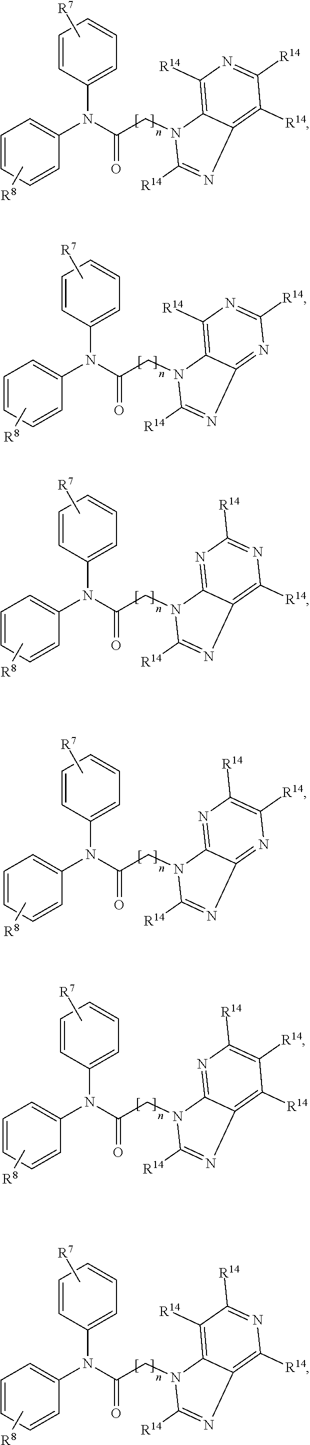

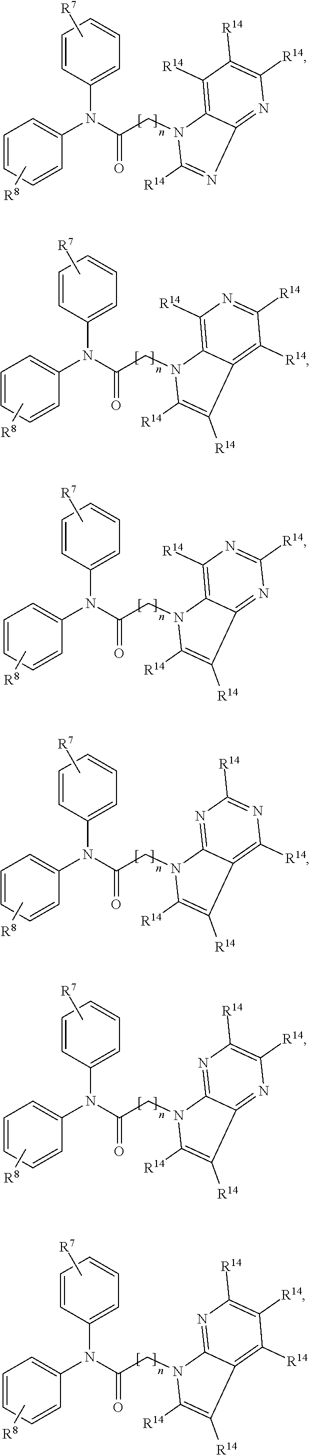

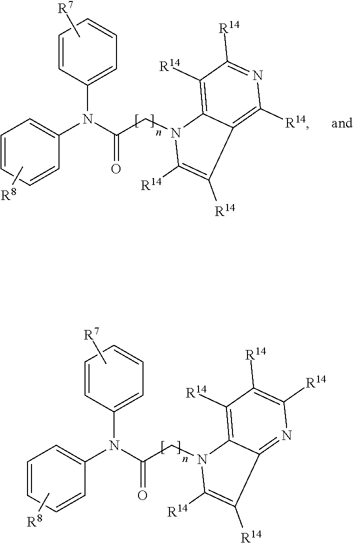

or a pharmaceutically acceptable salt, polymorph, prodrug, solvate or clathrate thereof.

In compounds of the formula (III), when R¹³ is a heterocyclyl of the formula (d), Y can be N. Alternatively, when R¹³ is a heterocyclyl of the formula (d), at least two of W, X, Y, and Z is N. Thus, for example, the compounds of the formula (III) can be compounds of the formulae:

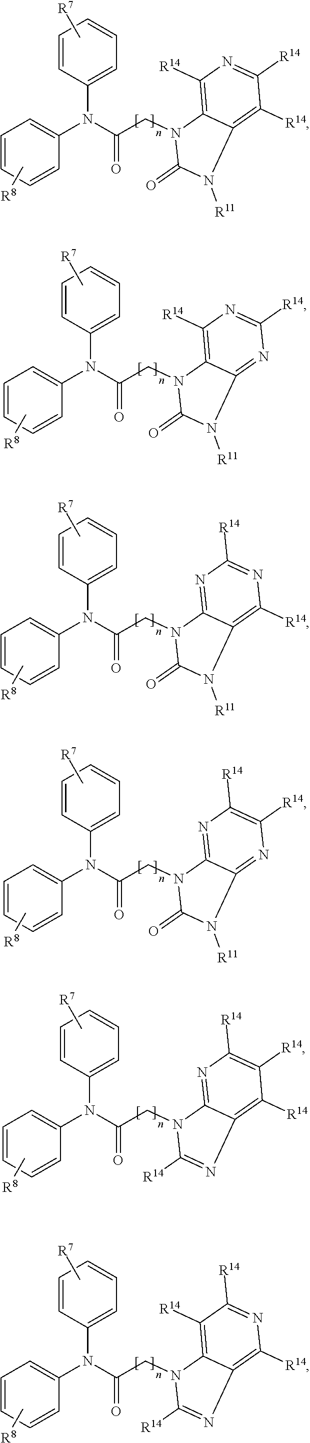

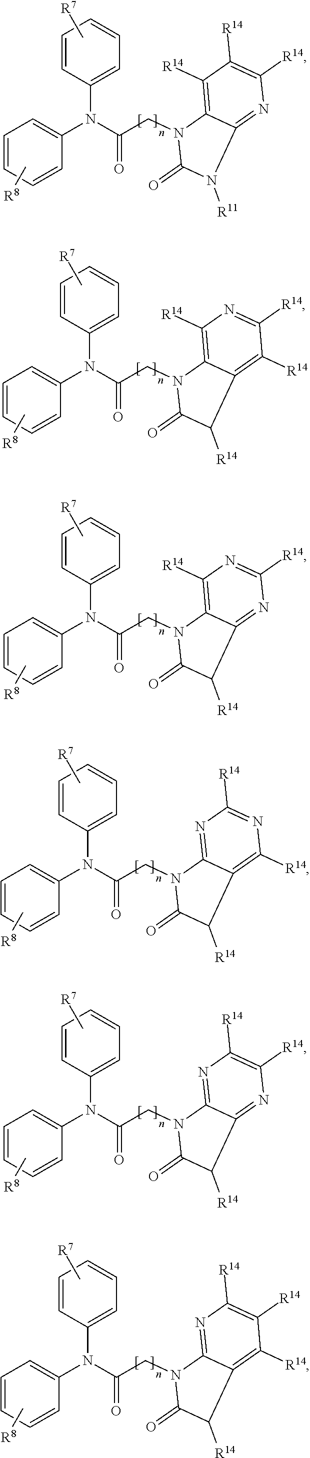

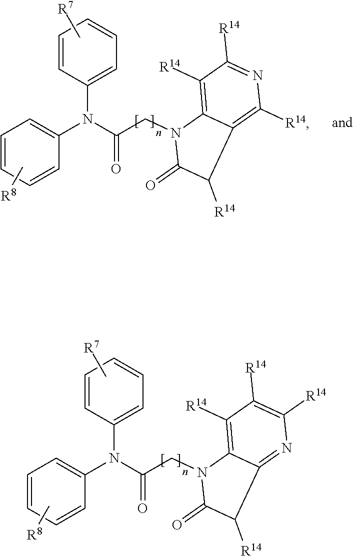

or a pharmaceutically acceptable salt, polymorph, prodrug, solvate or clathrate thereof.

In compounds of the formula (III), when R¹³ is a heterocyclyl of the formula (f), Y can be N. Alternatively, when R¹³ is a heterocyclyl of the formula (f), at least two of W, X, Y, and Z is N. Thus, for example, the compounds of the formula (III) can be compounds of the formulae:

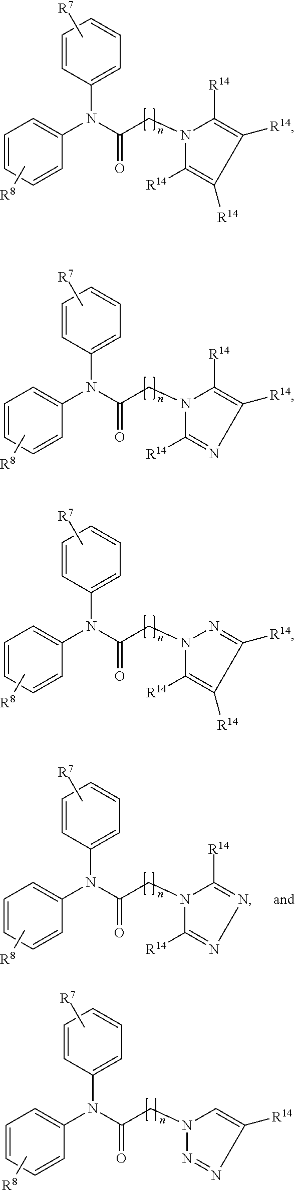

or a pharmaceutically acceptable salt, polymorph, prodrug, solvate or clathrate thereof.

The disclosure also relates to compounds of the formula (IV):

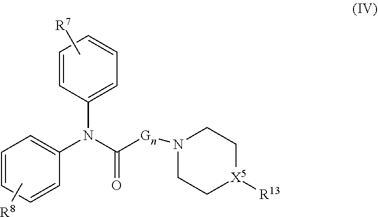

or a pharmaceutically acceptable salt, polymorph, prodrug, solvate or clathrate thereof,
wherein:
n is 0, 1 or 2;
each G is independently alkyl (e.g., CH₂) or C(O);
X⁵ is N or CR⁵, wherein R⁵ is hydrogen, alkyl, heterocyclyl or aryl;
R⁷ and R⁸ are each independently halo, a carbon with at least one halo (e.g., one to three halo, such as CHF₂, CCF₃, CCl₃), alkyl, aryl, acyl or heterocyclyl; and
R¹³ is a heterocyclyl group of the formula:

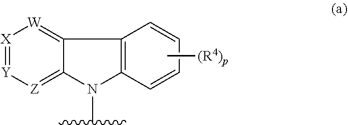

wherein W is N or C—R¹⁴; X is N or C—R¹⁴; Y is N or C—R¹⁴; and Z is N or C—R¹⁴; wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R⁴ is alkyl, cycloalkyl, aryl, heteroaryl, halo, amino, OR⁷, wherein R¹ is hydrogen, alkyl, cycloalkyl, aryl or arylalkyl, S(O)ₓ, wherein x is 0, 1 or 2, acyl, amido or heterocyclyl; and p is 0, 1, 2, 3 or 4;

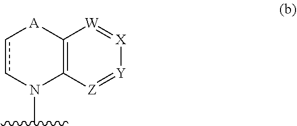

wherein A is S(O)ₓ, wherein x is 0, 1 or 2; O; C—R¹⁴, wherein R¹⁴ is hydrogen, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or A is N—R¹¹, wherein R¹¹ is a carbon with at least one halo (e.g., one to three halo, such as CHF₂, CCF₃, CCl₃), alkyl, aryl, acyl or heterocyclyl;

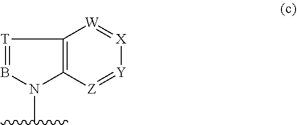

wherein T is CR¹⁴, wherein each R¹⁴ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or T is N; and
B is C—R¹⁴, wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or B is N;

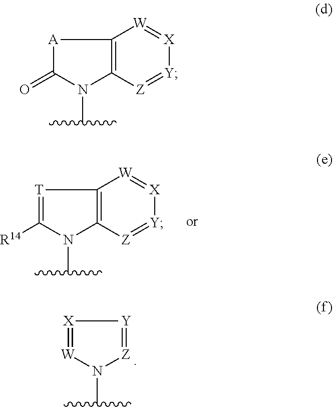

In compounds of the formula (IV), when R¹³ is a heterocyclyl of the formula (a), Y can be N. Alternatively, when R¹³ is a heterocyclyl of the formula (a), at least two of W, X, Y, and Z is N. Thus, for example, the compounds of the formula (IV) can be compounds of the formulae:

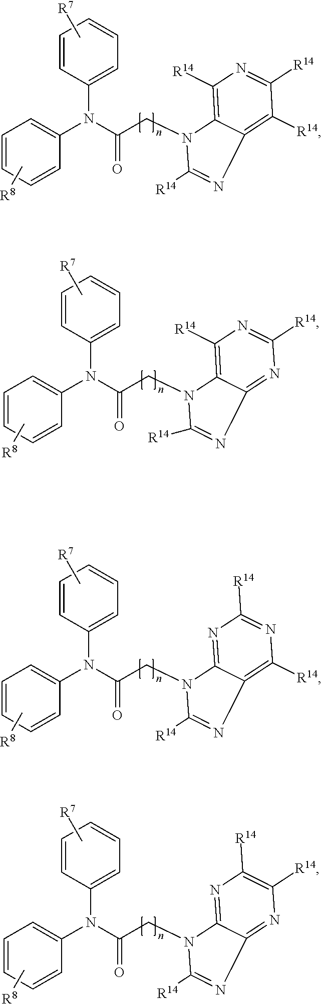

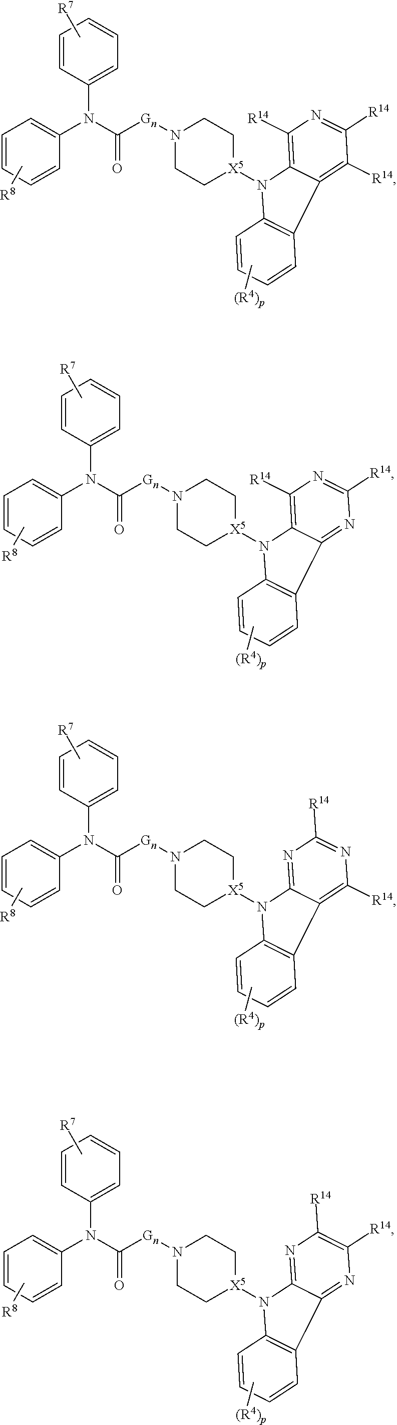

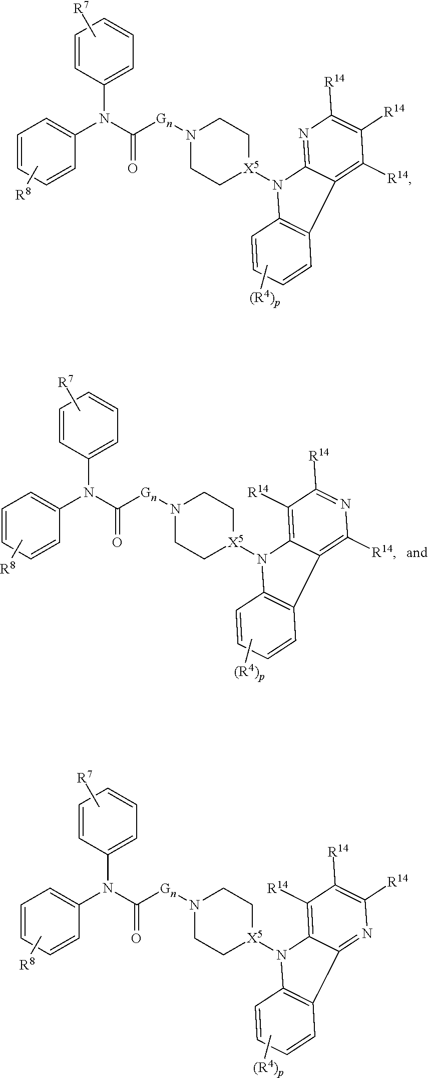

or a pharmaceutically acceptable salt, polymorph, prodrug, solvate or clathrate thereof.

In compounds of the formula (IV), when R¹³ is a heterocyclyl of the formula (b), Y can be N. Alternatively, when R¹³ is a heterocyclyl of the formula (b), at least two of W, X, Y, and Z is N. Thus, for example, the compounds of the formula (IV) can be compounds of the formulae:

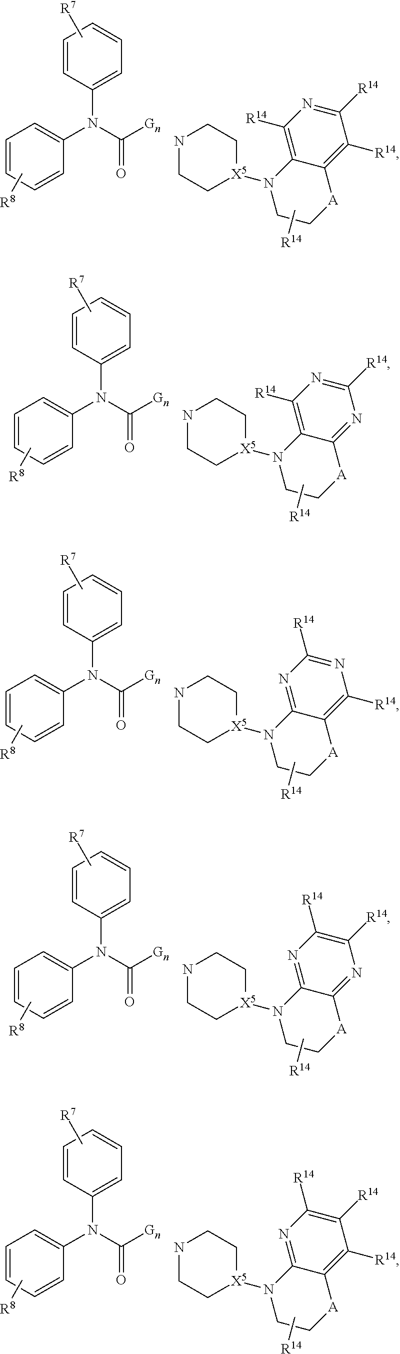

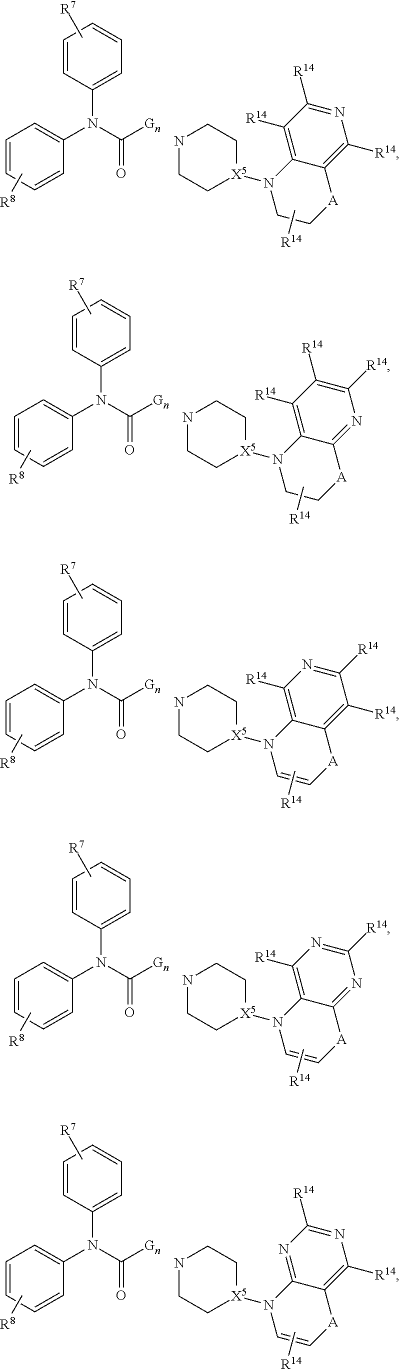

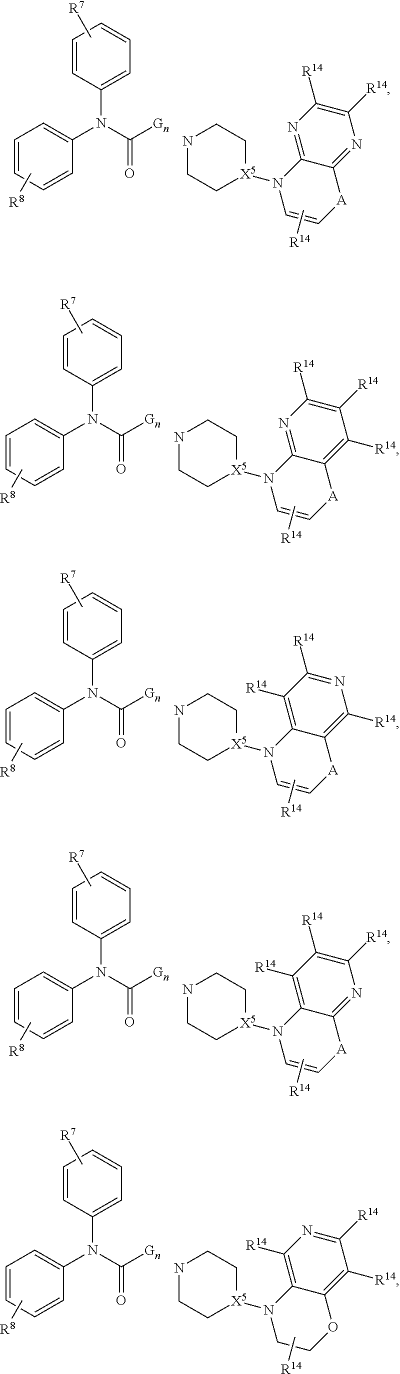

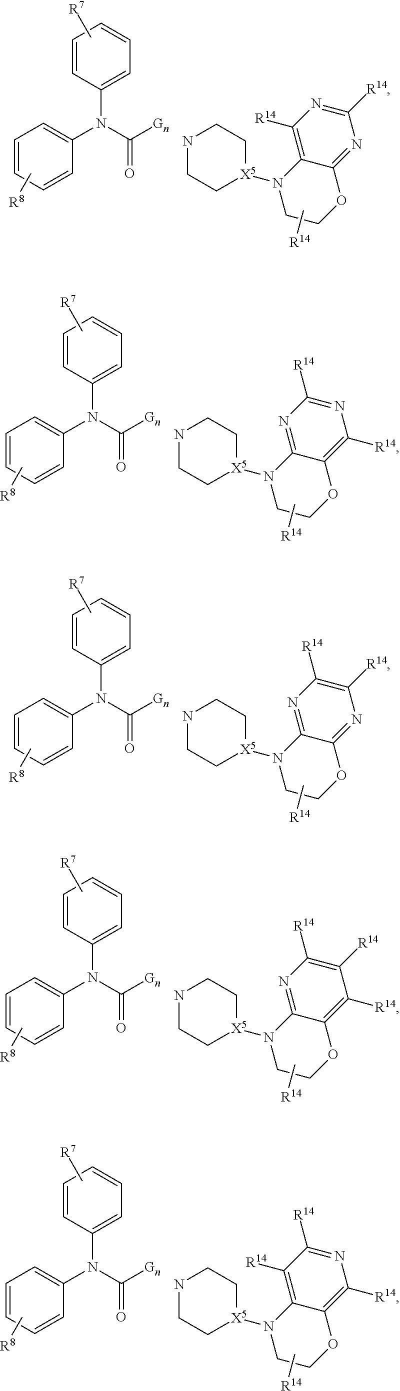

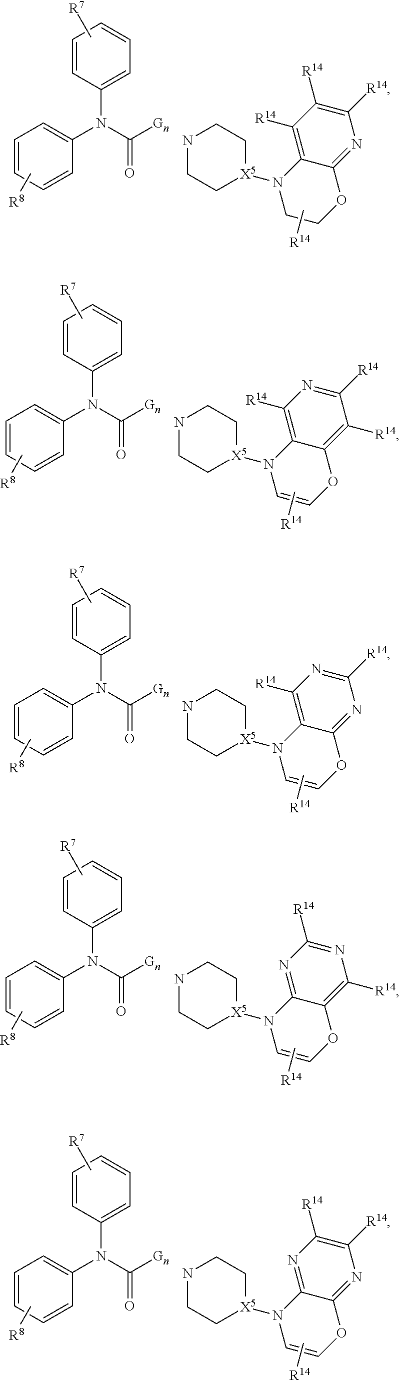

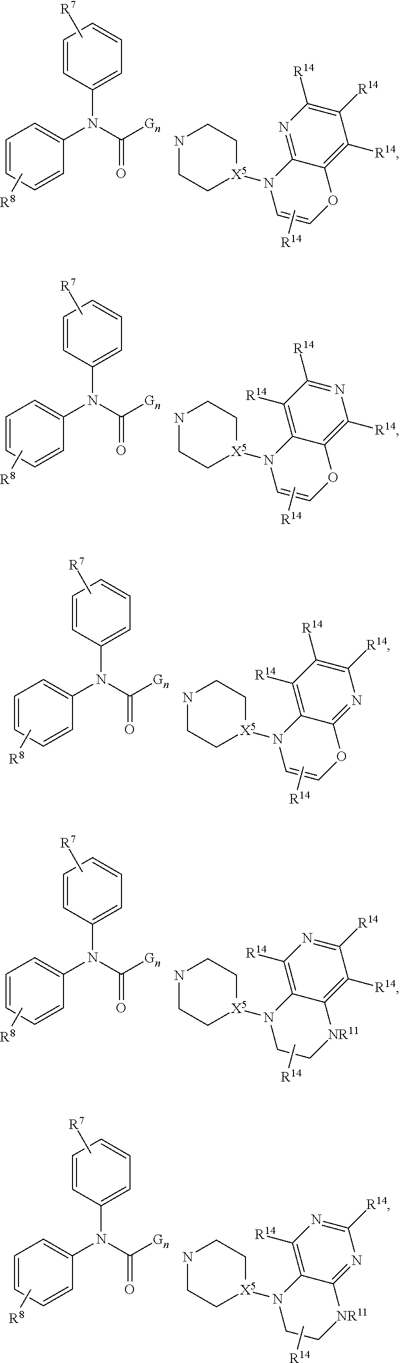

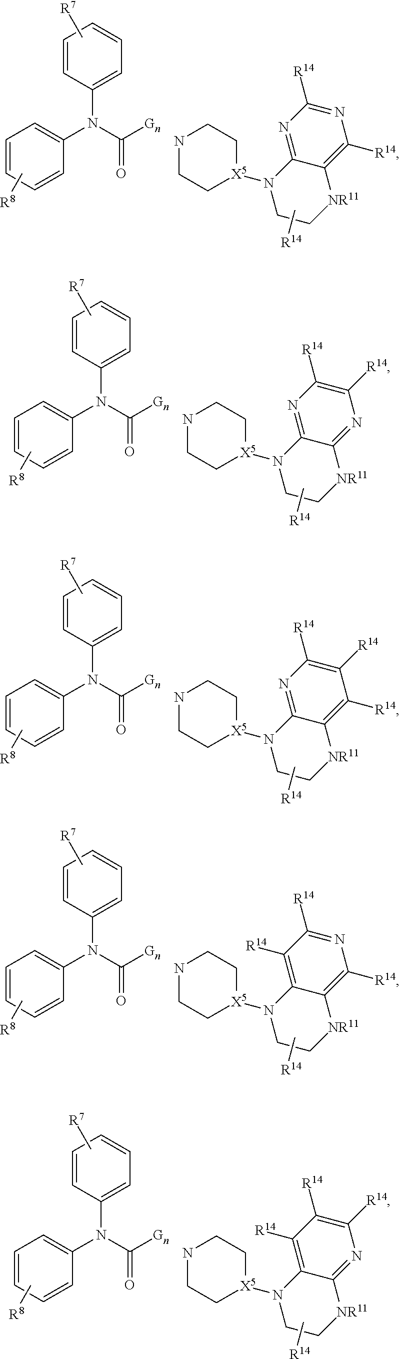

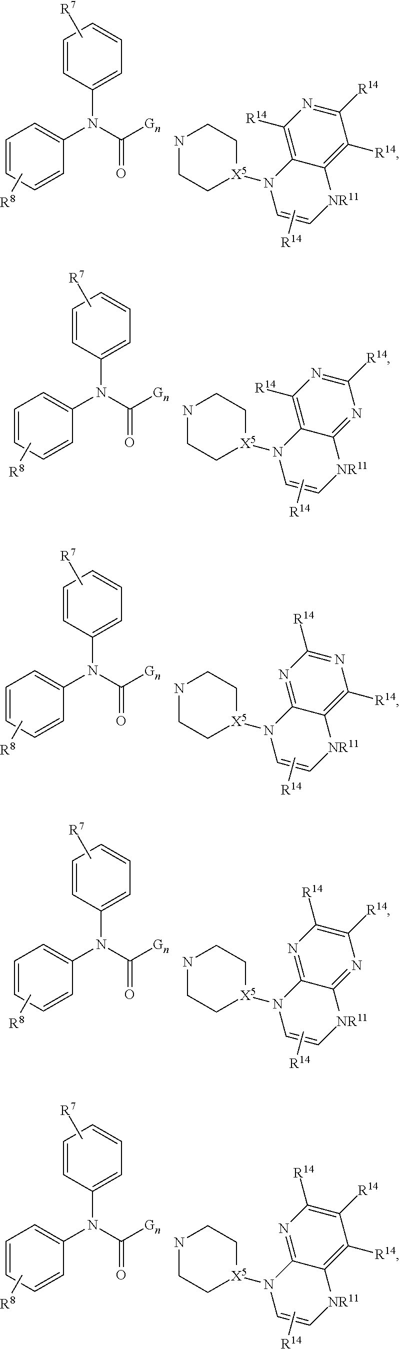

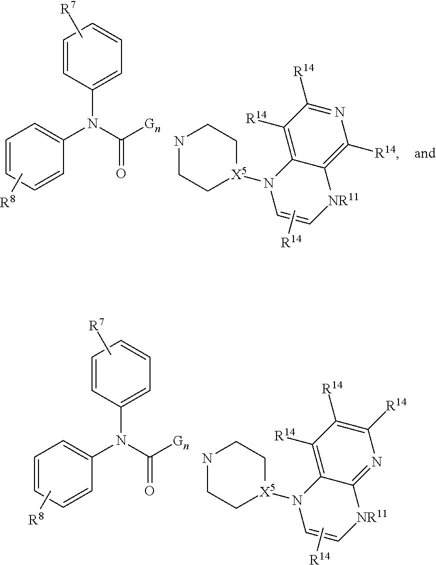

or a pharmaceutically acceptable salt, polymorph, prodrug, solvate or clathrate thereof.

In compounds of the formula (IV), when R¹³ is a heterocyclyl of the formula (c) or (e), Y can be N. Alternatively, when R¹³ is a heterocyclyl of the formula (c) or (e), at least two of W, X, Y, and Z is N. Thus, for example, the compounds of the formula (IV) can be compounds of the formulae:

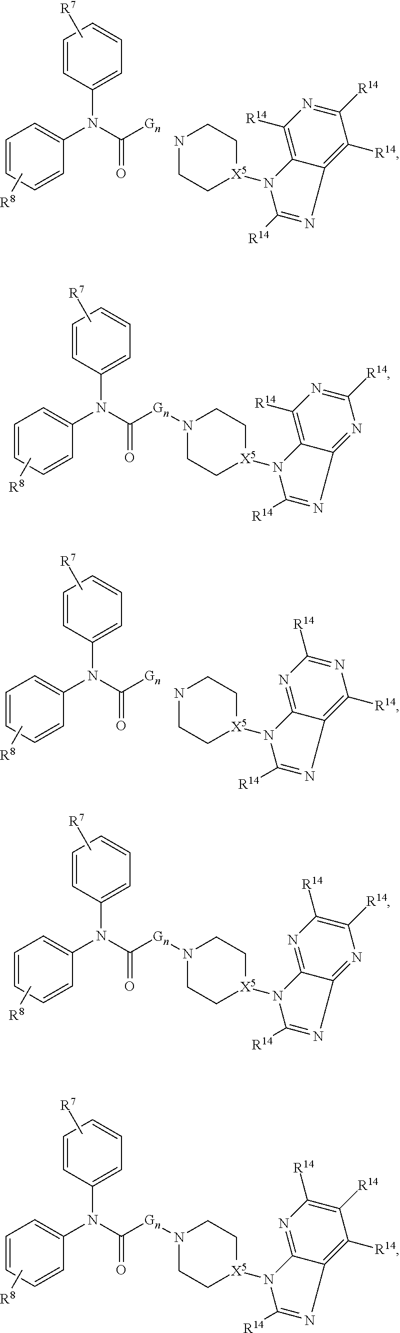

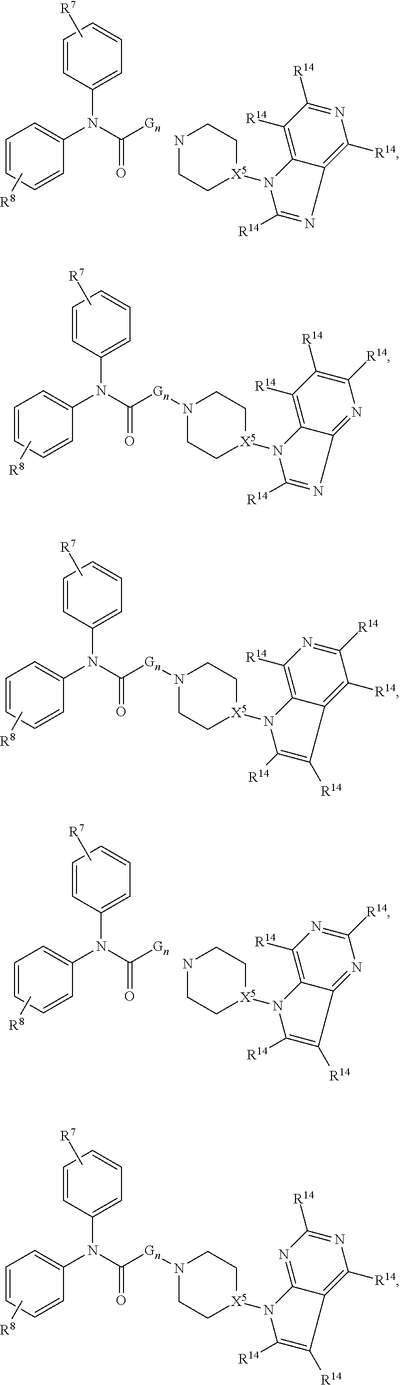

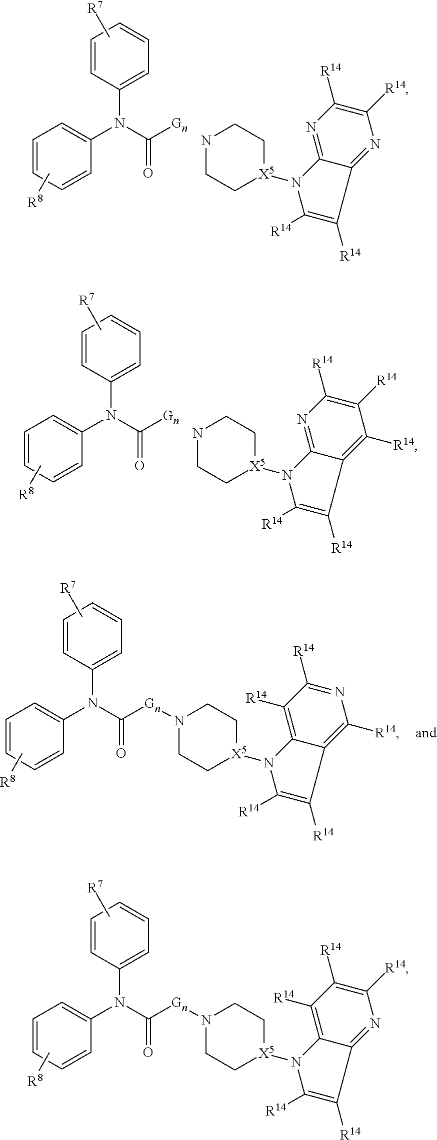

or a pharmaceutically acceptable salt, polymorph, prodrug, solvate or clathrate thereof.

In compounds of the formula (IV), when R¹³ is a heterocyclyl of the formula (d), Y can be N. Alternatively, when R¹³ is a heterocyclyl of the formula (d), at least two of W, X, Y, and Z is N. Thus, for example, the compounds of the formula (IV) can be compounds of the formulae:

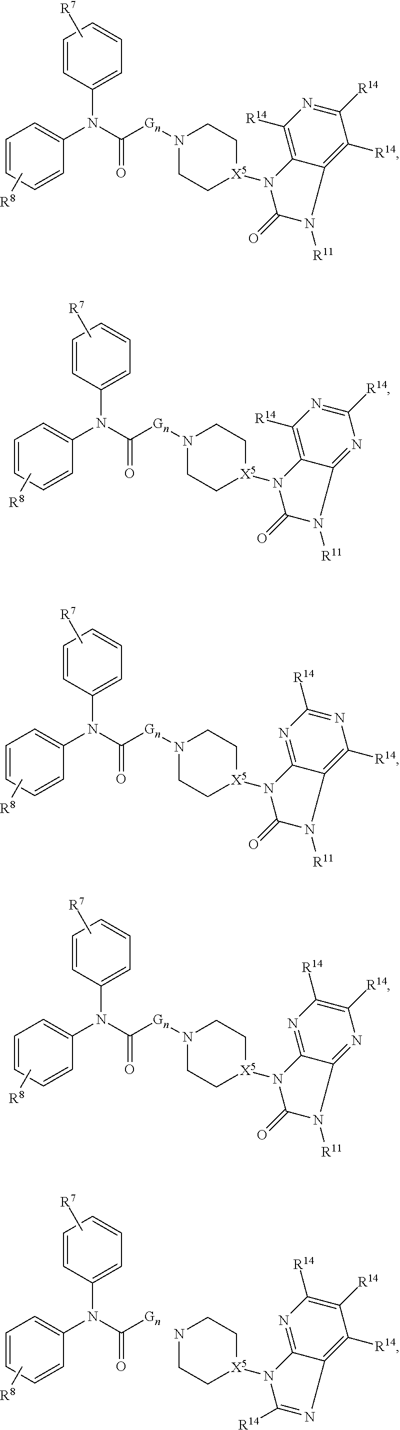

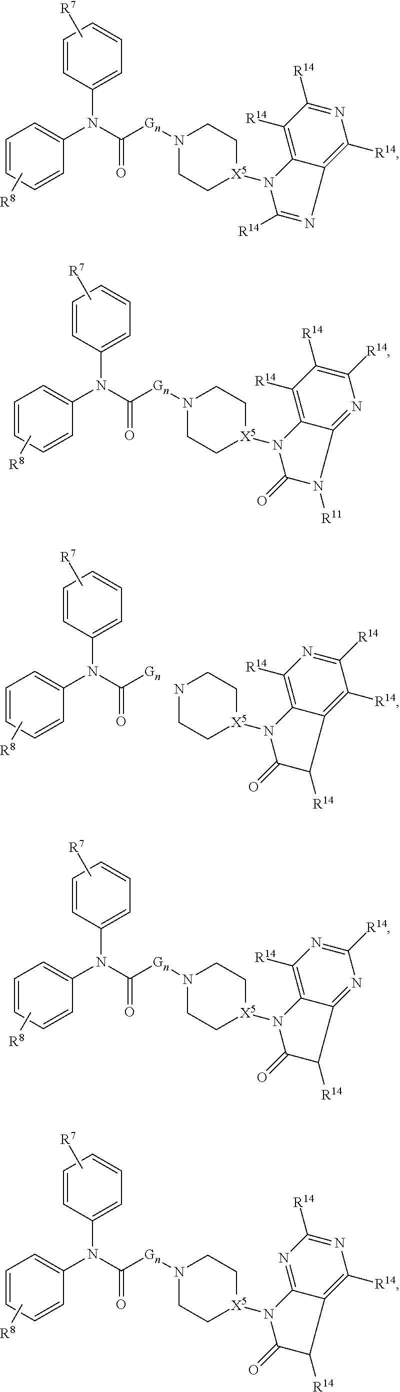

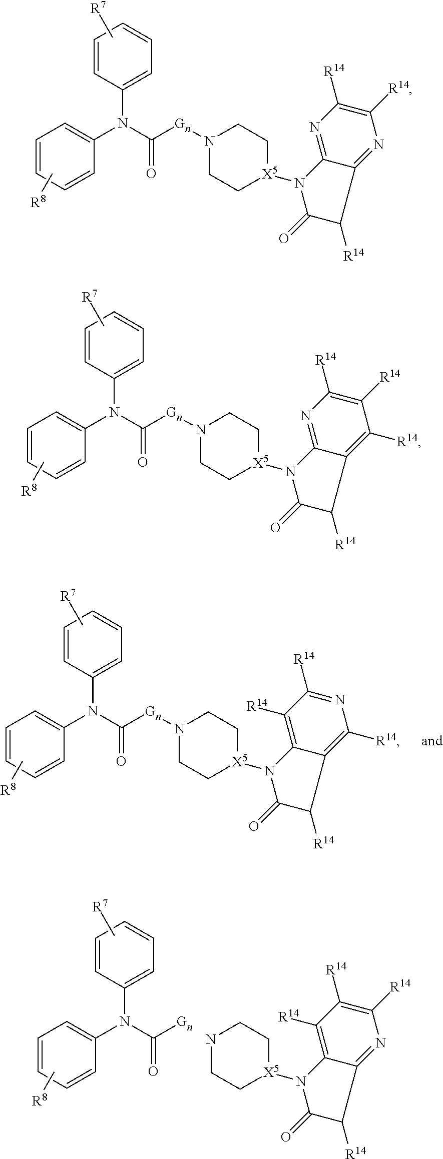

or a pharmaceutically acceptable salt, polymorph, prodrug, solvate or clathrate thereof.

In compounds of the formula (IV), when R¹³ is a heterocyclyl of the formula (f), Y can be N. Alternatively, when R¹³ is a heterocyclyl of the formula (f), at least two of W, X, Y, and Z is N. Thus, for example, the compounds of the formula (IV) can be compounds of the formulae:

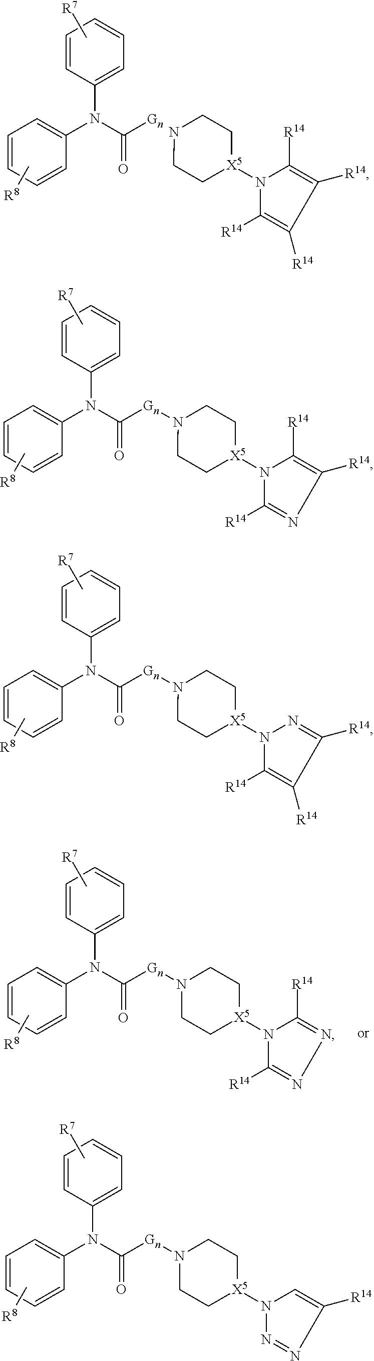

or a pharmaceutically acceptable salt, polymorph, prodrug, solvate or clathrate thereof.

The disclosure also relates to compounds of the formula (V):

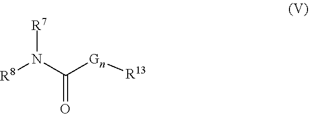

or a pharmaceutically acceptable salt, polymorph, prodrug, solvate or clathrate thereof,
wherein:
n is 0, 1 or 2;
each G is independently alkyl (e.g., CH₂) or C(O);
R⁷ and R⁸ are each independently halo, a carbon with at least one halo (e.g., one to three halo, such as CHF₂, CCF₃, CCl₃), alkyl, aryl, acyl or heterocyclyl; or
R⁷ and R⁸, together with the nitrogen atom to which they are attached, can form a heterocyclyl; and
R¹³ is a heterocyclyl group of the formula:

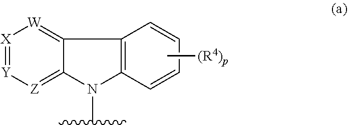

wherein W is N or C—R¹⁴; X is N or C—R¹⁴; Y is N or C—R¹⁴; and Z is N or C—R¹⁴;
wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R⁴ is alkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4;

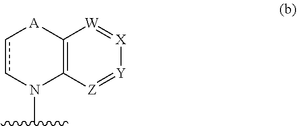

wherein A is S(O)ₓ, wherein x is 0, 1 or 2; O; C—R¹⁴, wherein R¹⁴ is hydrogen, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or A is N—R¹¹, wherein R¹ is a carbon with at least one halo (e.g., one to three halo, such as CHF₂, CCF₃, CCl₃), alkyl, aryl, acyl or heterocyclyl;

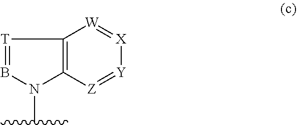

wherein T is CR¹⁴, wherein each R¹⁴ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or T is N; and
B is C—R¹⁴, wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or aryalkyl, S(O)ₓ, wherein x is 0, 1 or 2, acyl, amido or heterocyclyl; or B is N;

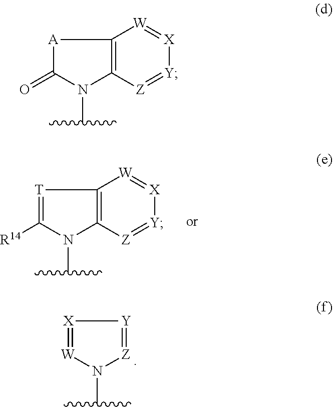

In compounds of the formula (V), when R¹³ is a heterocyclyl of the formula (a), Y can be N. Alternatively, when R¹³ is a heterocyclyl of the formula (a), at least two of W, X, Y, and Z is N. Thus, for example, the compounds of the formula (V) can be compounds of the formulae:

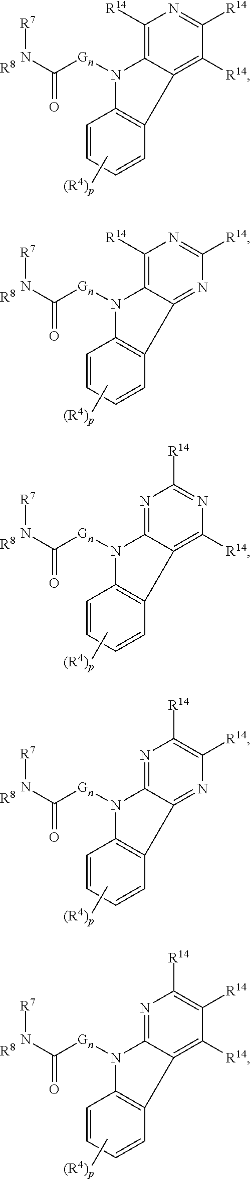

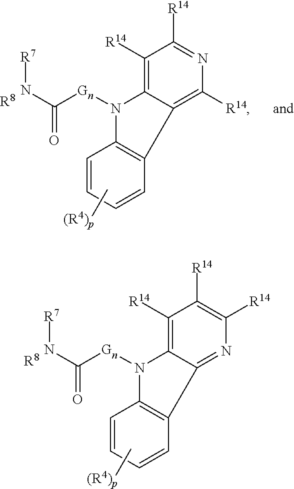

or a pharmaceutically acceptable salt, polymorph, prodrug, solvate or clathrate thereof.

In compounds of the formula (V), when R¹³ is a heterocyclyl of the formula (b), Y can be N. Alternatively, when R¹³ is a heterocyclyl of the formula (b), at least two of W, X, Y, and Z is N. Thus, for example, the compounds of the formula (V) can be compounds of the formulae:

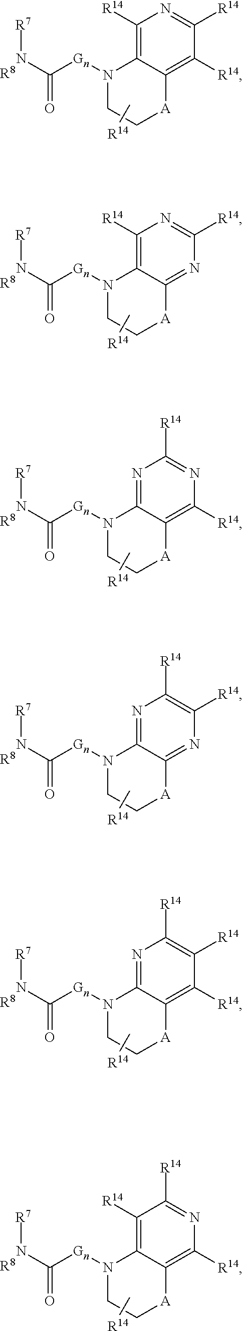

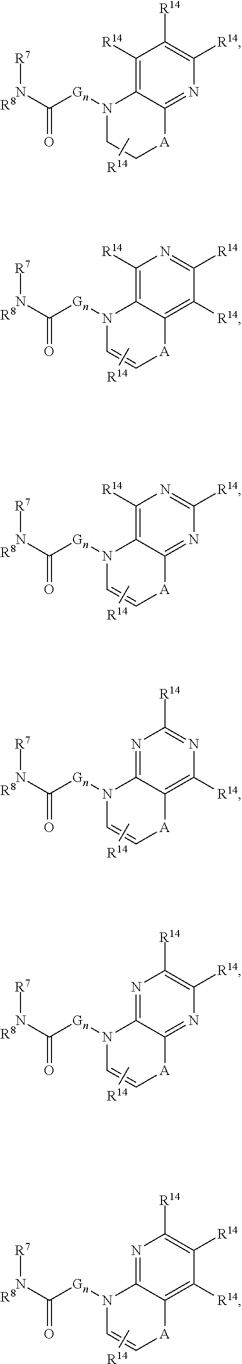

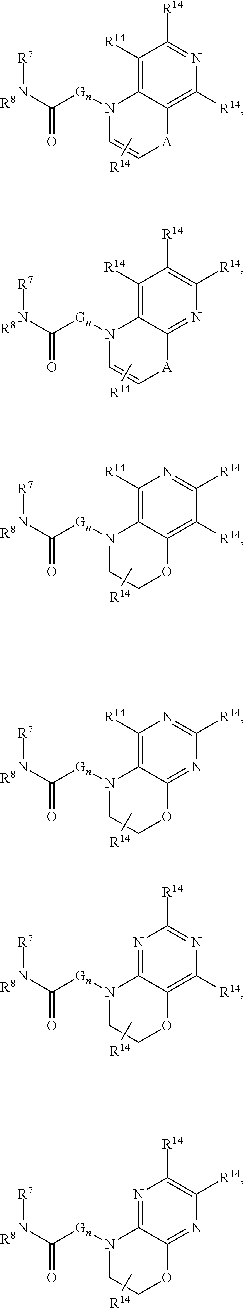

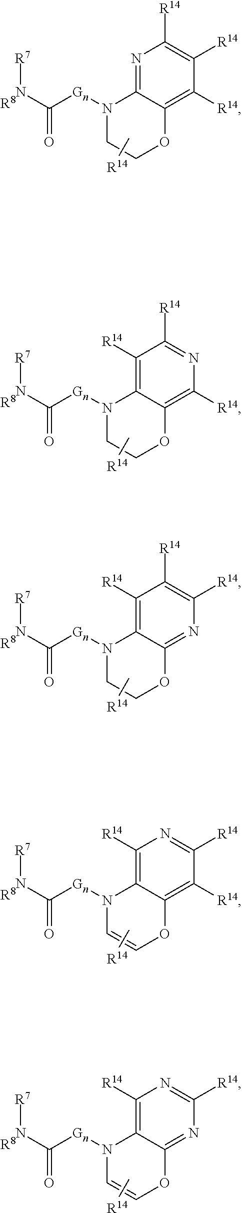

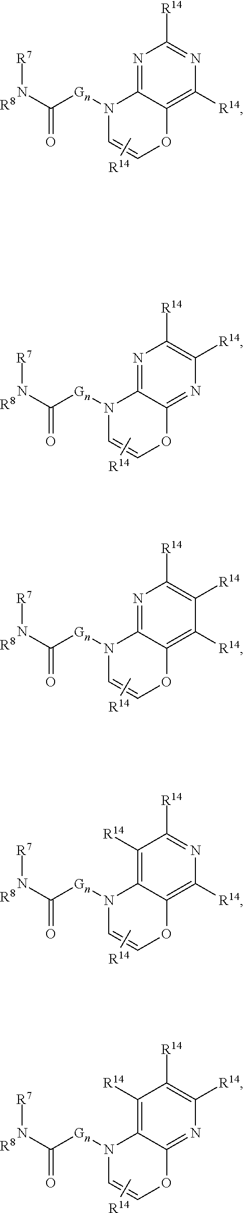

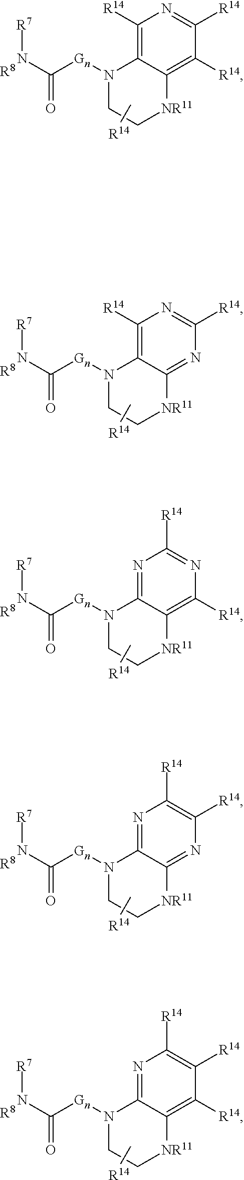

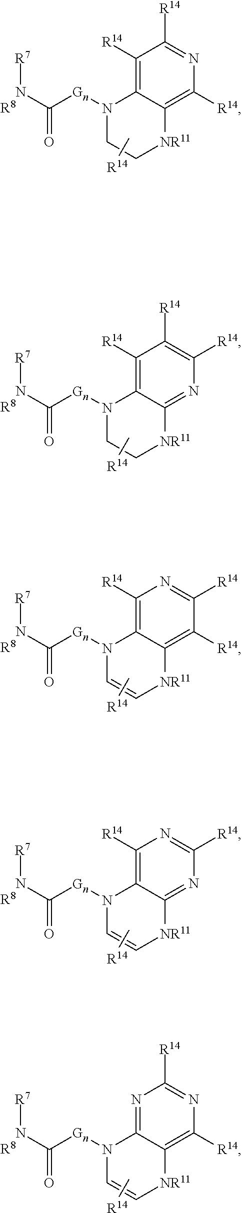

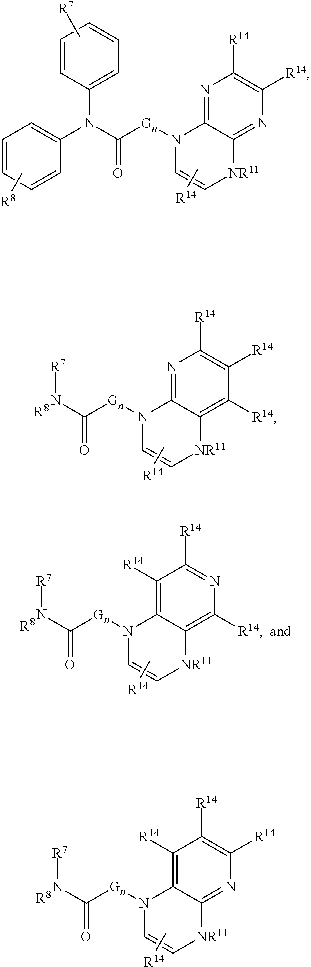

or a pharmaceutically acceptable salt, polymorph, prodrug, solvate or clathrate thereof.

In compounds of the formula (V), when R¹³ is a heterocyclyl of the formula (c) or (e), Y can be N. Alternatively, when R¹³ is a heterocyclyl of the formula (c) or (e), at least two of W, X, Y, and Z is N. Thus, for example, the compounds of the formula (V) can be compounds of the formulae:

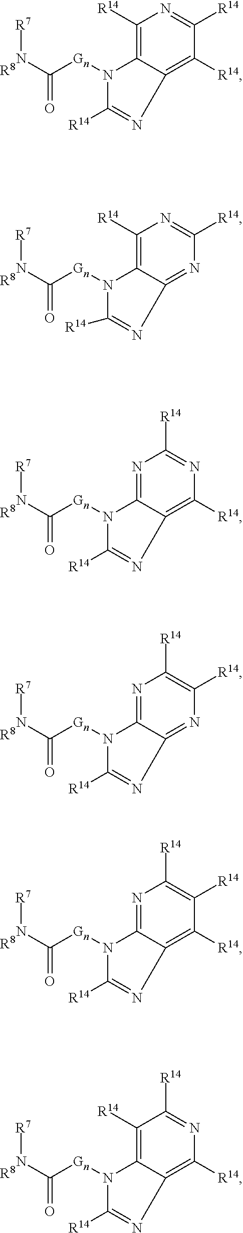

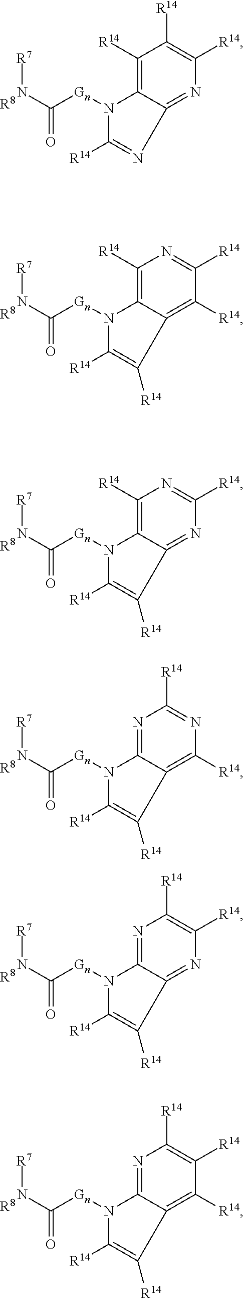

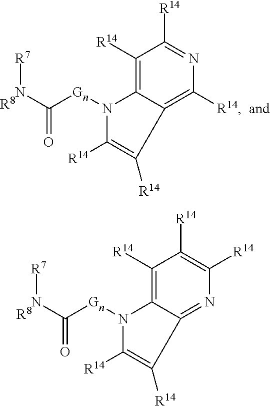

or a pharmaceutically acceptable salt, polymorph, prodrug, solvate or clathrate thereof.

In compounds of the formula (V), when R¹³ is a heterocyclyl of the formula (d), Y can be N. Alternatively, when R¹³ is a heterocyclyl of the formula (d), at least two of W, X, Y, and Z is N. Thus, for example, the compounds of the formula (V) can be compounds of the formulae:

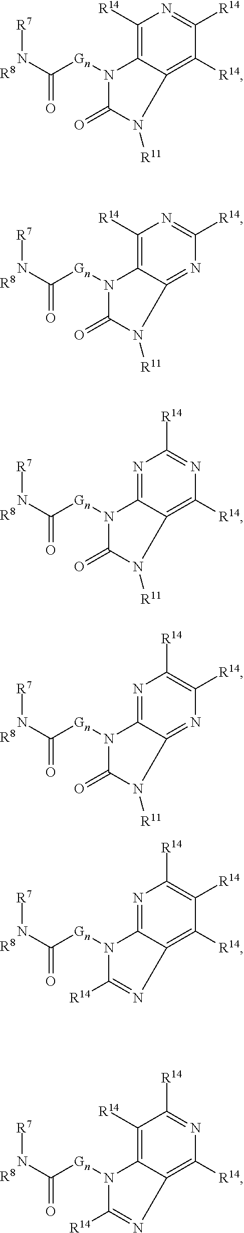

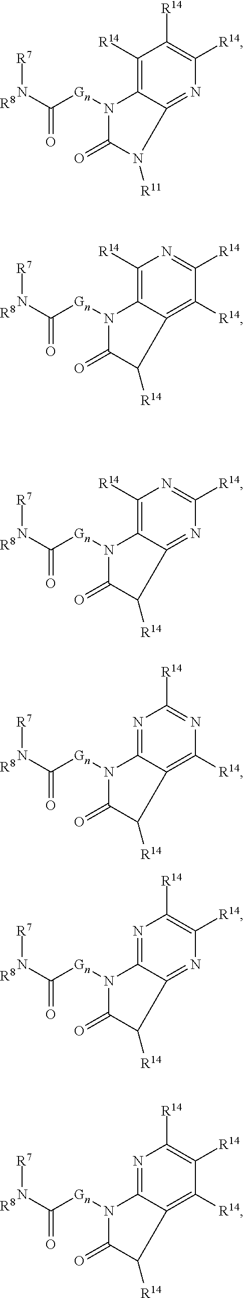

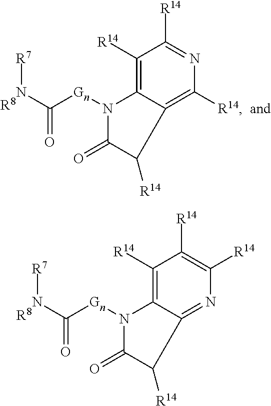

or a pharmaceutically acceptable salt, polymorph, prodrug, solvate or clathrate thereof.

In compounds of the formula (V), when R¹³ is a heterocyclyl of the formula (f), Y can be N. Alternatively, when R¹³ is a heterocyclyl of the formula (f), at least two of W, X, Y, and Z is N. Thus, for example, the compounds of the formula (V) can be compounds of the formulae:

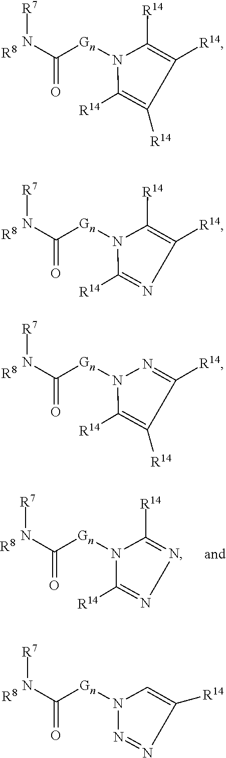

or a pharmaceutically acceptable salt, polymorph, prodrug, solvate or clathrate thereof.

The disclosure also relates to compounds of the formula (VI):

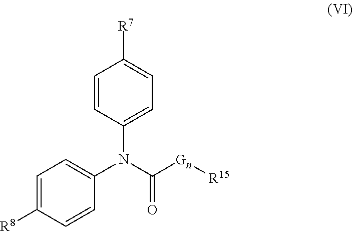

or a pharmaceutically acceptable salt, polymorph, prodrug, solvate or clathrate thereof,
wherein:
n is 0, 1 or 2;
each G is independently alkyl (e.g., CH₂) or C(O); and
R⁷ and R⁸ are each independently halo, a carbon with at least one halo (e.g., one to three halo, such as CHF₂, CCF₃, CCl₃), alkyl, aryl, acyl or heterocyclyl; and
R¹⁵ is a heterocyclyl group of the formula:

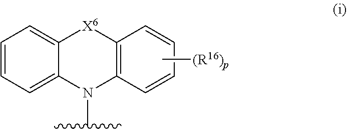

wherein X⁶ is alkyl (e.g., CH₂ and CH₂CH₂), alkenyl (e.g., CH═CH), S, O or NR¹⁷; wherein R¹⁷ is hydrogen, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or aryalkyl, S(O)ₓ, wherein x is 0, 1 or 2, acyl, amido or heterocyclyl;
R¹⁶ is alkyl, haloalkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4;

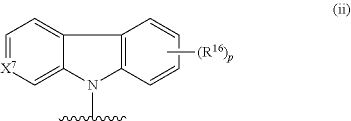

wherein X⁷ is N or C—R¹⁸; wherein R¹⁸ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and
R¹⁶ is alkyl, haloalkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4;

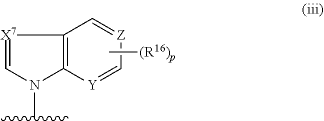

wherein X⁷ is N or C—R¹⁸; wherein R¹⁸ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R¹⁶ is alkyl, haloalkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4;
Y is N or C—R¹⁴; and Z is N or C—R¹⁴; wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;

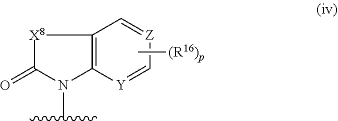

wherein X⁸ is NR¹⁹, wherein R¹⁹ is H, alkyl or aryl or X⁸ is C(R¹⁸)₂; wherein each R¹⁸ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R¹⁶ is alkyl, haloalkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4;
Y is N or C—R¹⁴; and Z is N or C—R¹⁴; wherein R⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;

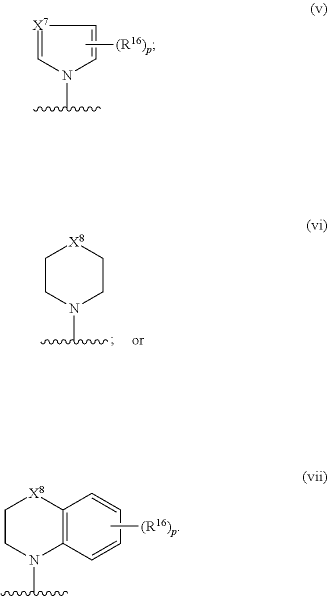

Examples of R¹⁵ groups of the formula (i) include:

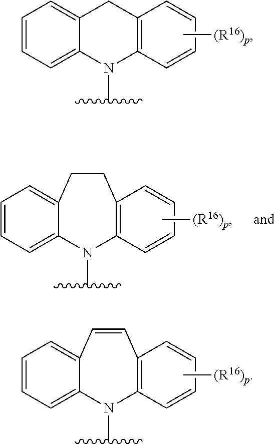

Examples of compounds of the formula (VI) include, but are not limited to, compounds of the formulae:

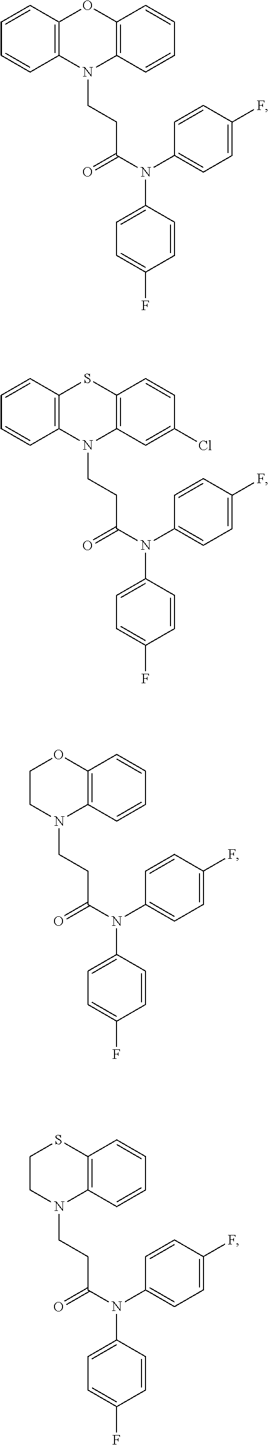 [SMILES: O=C(CCN1c2ccccc2Oc2ccccc21)N(c1ccc(F)cc1)c1ccc(F)cc1 ; O=C(CCN1c2ccccc2Sc2ccc(Cl)cc21)N(c1ccc(F)cc1)c1ccc(F)cc1 ; O=C(CCN1CCOc2ccccc21)N(c1ccc(F)cc1)c1ccc(F)cc1 ; O=C(CCN1CCSc2ccccc21)N(c1ccc(F)cc1)c1ccc(F)cc1]

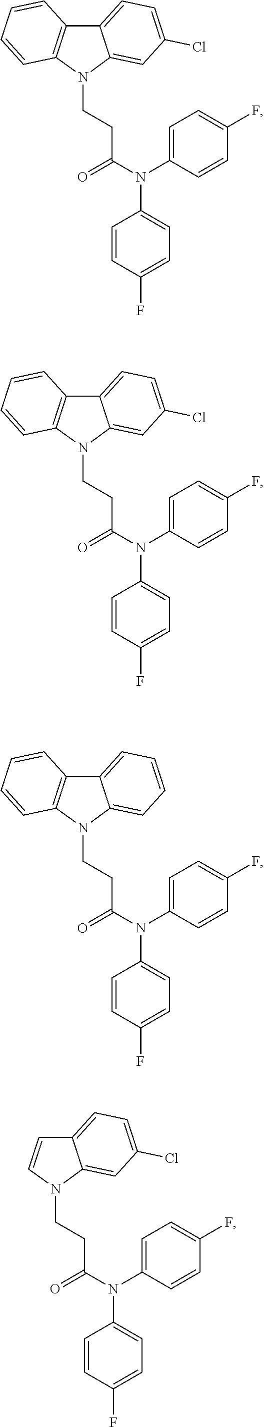 [SMILES: O=C(CCn1c2ccccc2c2ccc(Cl)cc21)N(c1ccc(F)cc1)c1ccc(F)cc1 ; O=C(CCn1c2ccccc2c2ccc(Cl)cc21)N(c1ccc(F)cc1)c1ccc(F)cc1 ; O=C(CCn1c2ccccc2c2ccccc21)N(c1ccc(F)cc1)c1ccc(F)cc1 ; O=C(CCn1ccc2ccc(Cl)cc21)N(c1ccc(F)cc1)c1ccc(F)cc1]

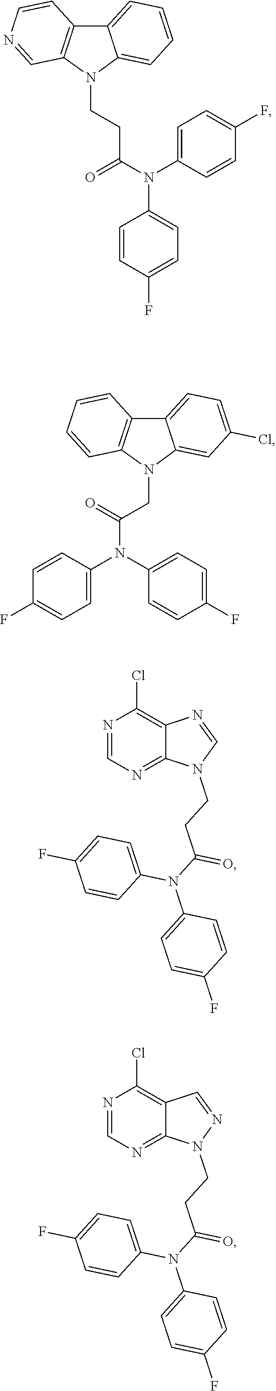 [SMILES: O=C(CCn1c2ccccc2c2ccncc21)N(c1ccc(F)cc1)c1ccc(F)cc1 ; O=C(Cn1c2ccccc2c2ccc(Cl)cc21)N(c1ccc(F)cc1)c1ccc(F)cc1 ; O=C(CCn1cnc2c(Cl)ncnc21)N(c1ccc(F)cc1)c1ccc(F)cc1 ; O=C(CCn1ncc2c(Cl)ncnc21)N(c1ccc(F)cc1)c1ccc(F)cc1]

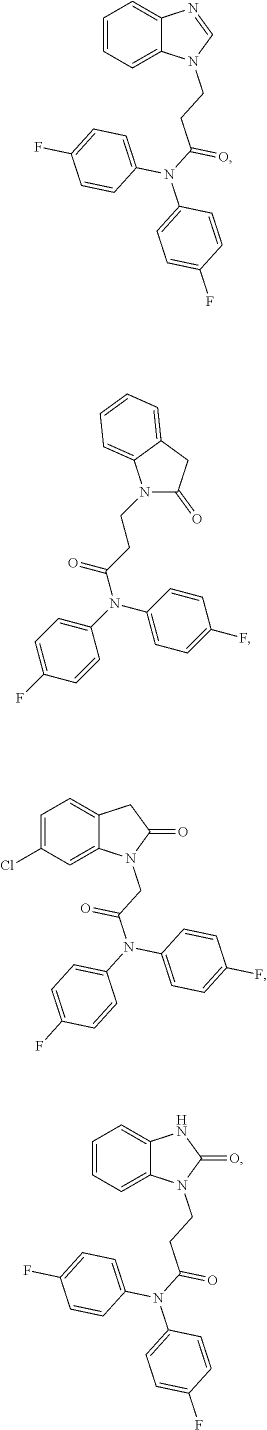 [SMILES: O=C(CCn1cnc2ccccc21)N(c1ccc(F)cc1)c1ccc(F)cc1 ; O=C1Cc2ccccc2N1CCC(=O)N(c1ccc(F)cc1)c1ccc(F)cc1 ; O=C1Cc2ccc(Cl)cc2N1CC(=O)N(c1ccc(F)cc1)c1ccc(F)cc1 ; O=C(CCn1c(=O)[nH]c2ccccc21)N(c1ccc(F)cc1)c1ccc(F)cc1]

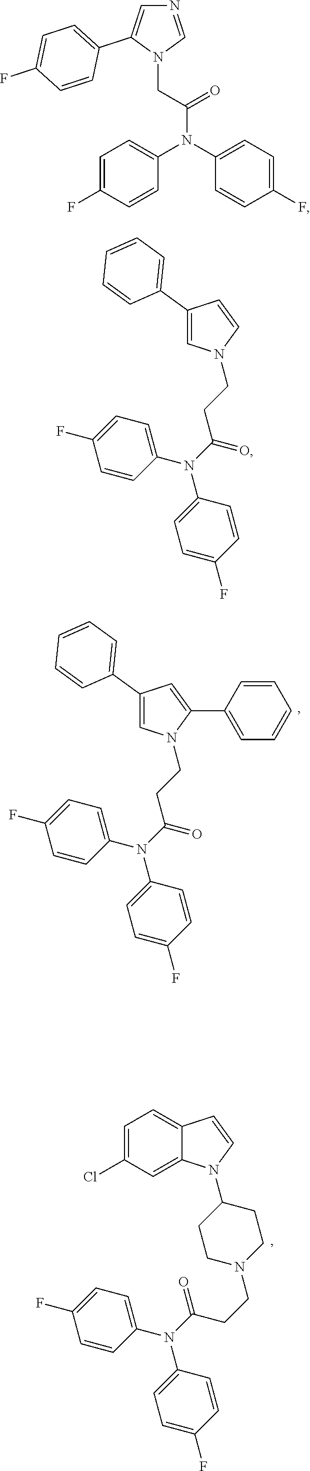

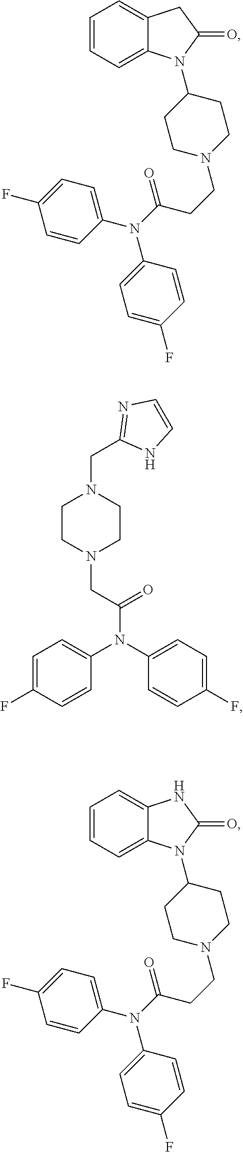

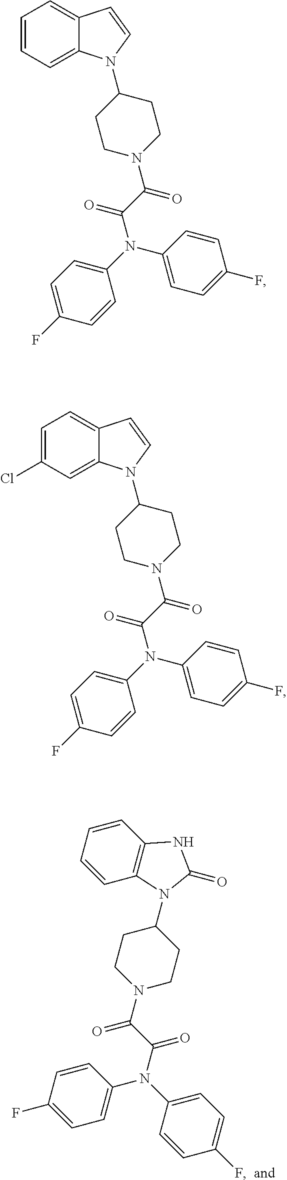

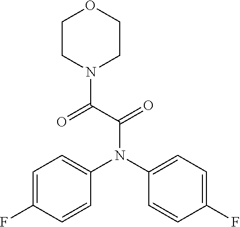 [SMILES: O=C(C(=O)N(c1ccc(F)cc1)c1ccc(F)cc1)N1CCOCC1]

or a pharmaceutically acceptable salt, polymorph, prodrug, solvate or clathrate thereof.

This disclosure also contemplates pharmaceutical compositions comprising one or more compounds and one or more pharmaceutically acceptable excipients. A “pharmaceutical composition” refers to a chemical or biological composition suitable for administration to a subject (e.g., mammal). Such compositions can be specifically formulated for administration via one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” is a carrier, sometimes a liquid, in which an active therapeutic agent is formulated. The excipient generally does not provide any pharmacological activity to the formulation, though it can provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes, but is not limited to, any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, the compounds described herein can be formulated in a time release formulation, for example in a composition that includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical compositions of the present invention can be orally administered as a capsule (hard or soft), tablet (film coated, enteric coated or uncoated), powder or granules (coated or uncoated) or liquid (solution or suspension). The formulations can be conveniently prepared by any of the methods well-known in the art. The pharmaceutical compositions of the present invention can include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition according to various embodiments of the present invention can vary according to factors such as the disease state, age, gender, weight, patient history, risk factors, predisposition to disease, administration route, pre-existing treatment regime (e.g., possible interactions with other medications), and weight of the subject. Dosage regimens can be adjusted to provide the optimum therapeutic response. For example, a single bolus can be administered, several divided doses can be administered over time, or the dose can be proportionally reduced or increased as indicated by the exigencies of therapeutic situation.

A “dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in subjects. In therapeutic use for treatment of conditions in mammals (e.g., humans) for which the compounds of the present invention or an appropriate pharmaceutical composition thereof are effective, the compounds of the present invention can be administered in an effective amount. The dosages as suitable for this invention can be a composition, a pharmaceutical composition or any other compositions described herein.

For each of the recited embodiments, the dosage is typically administered once, twice, or thrice a day, although more frequent dosing intervals are possible. The dosage can be administered every day, every 2 days, every 3 days, every 4 days, every 5 days, every 6 days, and/or every 7 days (once a week). In one embodiment, the dosage can be administered daily for up to and including 30 days, preferably between 7-10 days. In another embodiment, the dosage can be administered twice a day for 10 days. If the patient requires treatment for a chronic disease or condition, the dosage can be administered for as long as signs and/or symptoms persist. The patient can require “maintenance treatment” where the patient is receiving dosages every day for months, years, or the remainder of their lives. In addition, the composition of this invention can be to effect prophylaxis of recurring symptoms. For example, the dosage can be administered once or twice a day to prevent the onset of symptoms in patients at risk, especially for asymptomatic patients.

The absolute weight of a given compound included in a unit dose for administration to a subject can vary widely. For example, about 0.0001 to about 1 g, or about 0.001 to about 0.5 g, of at least one compound of this disclosure, or a plurality of compounds can be administered. Alternatively, the unit dosage can vary from about 0.001 g to about 2 g, from about 0.005 g to about 0.5 g, from about 0.01 g to about 0.25 g, from about 0.02 g to about 0.2 g, from about 0.03 g to about 0.15 g, from about 0.04 g to about 0.12 g, or from about 0.05 g to about 0.1 g.

Daily doses of the compounds can vary as well. Such daily doses can range, for example, from about 0.01 g/day to about 10 g/day, from about 0.02 g/day to about 5 g/day, from about 0.03 g/day to about 4 g/day, from about 0.04 g/day to about 3 g/day, from about 0.05 g/day to about 2 g/day, and from about 0.05 g/day to about 1 g/day.

It will be appreciated that the amount of compound(s) for use in treatment will vary not only with the particular carrier selected but also with the route of administration, the nature of the condition being treated, and the age and condition of the patient. Ultimately the attendant health care provider may determine proper dosage.

The compositions described herein can be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to, for example, tissue, organ, and/or organ system, which encompasses and/or is affected by the disease, and/or where the disease signs and/or symptoms are active or are likely to occur. Administration can be topical with a local effect, composition is applied directly where its action is desired. Administration can be enteral wherein the desired effect is systemic (non-local), composition is given via the digestive tract. Administration can be parenteral, where the desired effect is systemic, composition is given by other routes than the digestive tract.

The compositions can include the compounds described herein in a “therapeutically effective amount.” Such a therapeutically effective amount is an amount sufficient to obtain the desired physiological effect, such as a reduction of at least one symptom of cancer or an inflammatory disease or condition.

The compositions contemplated herein can contain other ingredients such as chemotherapeutic agents, anti-inflammatory agents, anti-viral agents, antibacterial agents, antimicrobial agents, immunomodulatory drugs, such as lenalidomide, pomalidomide or thalidomide, histone deacetylase inhibitors, such as panobinostat, preservatives or combinations thereof.

This disclosure also includes methods for treating neurodegenerative diseases, such as Parkinson's disease, Alzheimer's disease, Huntington's disease, and ALS, comprising administering a therapeutically effective amount of at least one of the compounds described herein (e.g., compounds of the formulae (I), (Ia)-(Ik), (II), (III), (IV), (V), and (VI)) to a subject in need thereof. This disclosure also includes methods for reducing, substantially eliminating or eliminating dysregulation of proteostasis comprising administering a therapeutically effective amount of at least one of the compounds described herein (e.g., compounds of the formulae (I), (Ia)-(Ik), (II), (III), (IV), (V), and (VI)) to a subject in need thereof. This disclosure also includes methods for reducing, substantially eliminating or eliminating the accumulation of intrinsically disordered proteins (e.g., α-syn) comprising administering a therapeutically effective amount of at least one of the compounds described herein (e.g., compounds of the formulae compounds of the formulae (I), (Ia)-(Ik), (II), (III), (IV), (V), and (VI)) to a subject in need thereof.

As used herein, the terms “treat” and “treating” are not limited to the case where the subject (e.g. patient) is cured and the disease is eradicated. Rather, treatment that merely reduces symptoms, and/or delays disease progression is also contemplated.

The pharmaceutical compositions disclosed herein can have the ability to effectively treat new patient segments where proteasome inhibition and reduced toxicity is desired or warranted.

The compounds and methods described herein can be used prophylactically or therapeutically. The term “prophylactic” or “therapeutic” treatment refers to administration of a drug to a host before or after onset of a disease or condition. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom). Administering the compounds described herein (including enantiomers and salts thereof) is contemplated in both a prophylactic treatment (e.g. to patients at risk for disease, such as elderly patients who, because of their advancing age, are at risk for arthritis, cancer, and the like) and therapeutic treatment (e.g. to patients with symptoms of disease or to patients diagnosed with disease).

The term “therapeutically effective amount” as used herein, refers to that amount of one or more compounds of the various examples of the present invention that elicits a biological or medicinal response in a tissue system, animal or human, that is being sought by a researcher, veterinarian, medical doctor or other clinician, which includes alleviation of the symptoms of the disease or disorder being treated. In some examples, the therapeutically effective amount is that which can treat or alleviate the disease or symptoms of the disease at a reasonable benefit/risk ratio applicable to any medical treatment. However, it is to be understood that the total daily usage of the compounds and compositions described herein can be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds described herein.

The term “alkyl” as used herein refers to substituted or unsubstituted straight chain, branched and cyclic, saturated mono- or bi-valent groups having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. Examples of straight chain bi-valent (C₁-C₂₀)alkyl groups include those with from 1 to 6 carbon atoms such as —CH₂—, —CH₂CH₂—, —CH₂CH₂CH₂—, —CH₂CH₂CH₂CH₂—, and —CH₂CH₂CH₂CH₂CH₂—. Examples of branched bi-valent alkyl groups include —CH(CH₃)CH₂— and —CH₂CH(CH₃)CH₂—. Examples of cyclic alkyl groups include cyclopropyl, cyclobutyl, cyclopently, cyclohexyl, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. In some embodiments, alkyl includes a combination of substituted and unsubstituted alkyl. As an example, alkyl, and also (C₁)alkyl, includes methyl and substituted methyl. As a particular example, (C₁)alkyl includes benzyl. As a further example, alkyl can include methyl and substituted (C₂-C₈)alkyl. Alkyl can also include substituted methyl and unsubstituted (C₂-C₈)alkyl. In some embodiments, alkyl can be methyl and C₂-C₈ linear alkyl. In some embodiments, alkyl can be methyl and C₂-C₈ branched alkyl. The term methyl is understood to be —CH₃, which is not substituted. The term methylene is understood to be —CH₂—, which is not substituted. For comparison, the term (C)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further example, representative substituted alkyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkyl groups can be substituted from a set of groups including amino, hydroxy, cyano, carboxy, nitro, thio and alkoxy, but not including halogen groups. Thus, in some embodiments alkyl can be substituted with a non-halogen group. For example, representative substituted alkyl groups can be substituted with a fluoro group, substituted with a bromo group, substituted with a halogen other than bromo, or substituted with a halogen other than fluoro. In some embodiments, representative substituted alkyl groups can be substituted with one, two, three or more fluoro groups or they can be substituted with one, two, three or more non-fluoro groups. For example, alkyl can be trifluoromethyl, difluoromethyl, or fluoromethyl, or alkyl can be substituted alkyl other than trifluoromethyl, difluoromethyl or fluoromethyl. Alkyl can be haloalkyl or alkyl can be substituted alkyl other than haloalkyl. The term “alkyl” also generally refers to alkyl groups that can comprise one or more heteroatoms in the carbon chain. Thus, for example, “alkyl” also encompasses groups such as —[(CH₂)ₚO]_{q}H and the like.

The term “alkenyl” as used herein refers to substituted or unsubstituted straight chain, branched and cyclic, saturated mono- or bi-valent groups having at least one carbon-carbon double bond and from 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. The double bonds can be be trans or cis orientation. The double bonds can be terminal or internal. The alkenyl group can be attached via the portion of the alkenyl group containing the double bond, e.g., vinyl, propen-1-yl and buten-1-yl, or the alkenyl group can be attached via a portion of the alkenyl group that does not contain the double bond, e.g., penten-4-yl. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl. Examples of straight chain bi-valent (C₂-C₂₀)alkenyl groups include those with from 2 to 6 carbon atoms such as —CHCH—, —CHCHCH₂—, —CHCHCH₂CH₂—, and —CHCHCH₂CH₂CH₂—. Examples of branched bi-valent alkyl groups include —C(CH₃)CH— and —CHC(CH₃)CH₂—. Examples of cyclic alkenyl groups include cyclopentenyl, cyclohexenyl and cyclooctenyl. It is envisaged that alkenyl can also include masked alkenyl groups, precursors of alkenyl groups or other related groups. As such, where alkenyl groups are described it, compounds are also envisaged where a carbon-carbon double bond of an alkenyl is replaced by an epoxide or aziridine ring. Substituted alkenyl also includes alkenyl groups which are substantially tautomeric with a non-alkenyl group. For example, substituted alkenyl can be 2-aminoalkenyl, 2-alkylaminoalkenyl, 2-hydroxyalkenyl, 2-hydroxyvinyl, 2-hydroxypropenyl, but substituted alkenyl is also understood to include the group of substituted alkenyl groups other than alkenyl which are tautomeric with non-alkenyl containing groups. In some embodiments, alkenyl can be understood to include a combination of substituted and unsubstituted alkenyl. For example, alkenyl can be vinyl and substituted vinyl. For example, alkenyl can be vinyl and substituted (C₃-C₈)alkenyl. Alkenyl can also include substituted vinyl and unsubstituted (C₃-C₈)alkenyl. Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further example, representative substituted alkenyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkenyl groups can be substituted from a set of groups including monoalkylamino, dialkylamino, cyano, acetyl, amido, carboxy, nitro, alkylthio and alkoxy, but not including halogen groups. Thus, in some embodiments alkenyl can be substituted with a non-halogen group. In some embodiments, representative substituted alkenyl groups can be substituted with a fluoro group, substituted with a bromo group, substituted with a halogen other than bromo, or substituted with a halogen other than fluoro. For example, alkenyl can be 1-fluorovinyl, 2-fluorovinyl, 1,2-difluorovinyl, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. In some embodiments, representative substituted alkenyl groups can be substituted with one, two, three or more fluoro groups or they can be substituted with one, two, three or more non-fluoro groups.

The term “alkynyl” as used herein, refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. Examples include, but are not limited to ethynyl, propynyl, propyn-1-yl, propyn-2-yl, butynyl, butyn-1-yl, butyn-2-yl, butyn-3-yl, butyn-4-yl, pentynyl, pentyn-1-yl, hexynyl, Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀)aryl encompasses mono- and polycyclic (C₆-C₂₀)aryl groups, including fused and non-fused polycyclic (C₆-C₂₀)aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise a≤C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

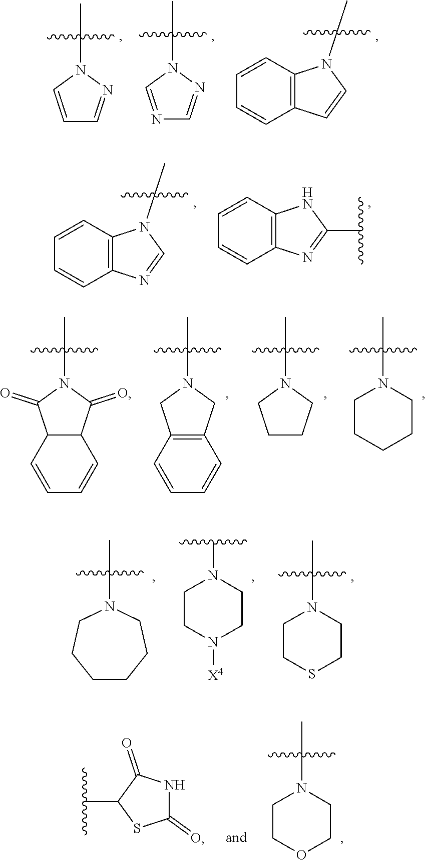

wherein X⁴ represents H, (C₁-C₂₀)alkyl, (C₆-C₂₀)aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. Thus, alkyoxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryloxy” as used herein refers to an oxygen atom connected to an aryl group as are defined herein.

The term “aralkyl” and “arylakyl” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The terms “halo,” “halogen,” or “halide” group, as used herein, by themselves or as part of another substituent, mean, unless otherwise stated, a fluorine, chlorine, bromine, or iodine atom.

The term “amine” and “amino” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “akylamino” group includes a monoalkylamino, dialkylamino, and trialkylamino group.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, aryl, cycloalkyl, heterocyclyl, group or the like.

The term “formyl” as used herein refers to a group containing a carbonyl moiety wherein the group is bonded via the carbonyl carbon atom. The carbonyl carbon atom is also bonded to a hydrogen atom.

The term “alkoxycarbonyl” as used herein refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom is also bonded to an oxygen atom which is further bonded to an alkyenyl group. Alkoxycarbonyl also includes the group where a carbonyl carbon atom is also bonded to an oxygen atom which is further bonded to an alkynyl group. In a further case, which is included in the definition of alkoxycarbonyl as the term is defined herein, and is also included in the term “aryloxycarbonyl,” the carbonyl carbon atom is bonded to an oxygen atom which is bonded to an aryl group instead of an alkyl group.

The term “arylcarbonyl” as used herein refers to a group containing a carbonyl moiety wherein the group is bonded via the carbonyl carbon atom. The carbonyl carbon atom is also bonded to an aryl group.

The term “alkylamido” as used herein refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylamido as the term is defined herein, the carbonyl carbon atom is bonded to a nitrogen atom which is bonded to one or more aryl group instead of, or in addition to, the one or more alkyl group. In a further case, which is also an alkylamido as the term is defined herein, the carbonyl carbon atom is bonded to an nitrogen atom which is bonded to one or more alkenyl group instead of, or in addition to, the one or more alkyl and or/aryl group. In a further case, which is also an alkylamido as the term is defined herein, the carbonyl carbon atom is bonded to a nitrogen atom which is bonded to one or more alkynyl group instead of, or in addition to, the one or more alkyl, alkenyl and/or aryl group.

The term “carboxy” as used herein refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “amido” or “amide” as used herein refers to a group having the formula C(O)NRR, wherein R is defined herein and can each independently be, e.g., hydrogen, alkyl, aryl or each R, together with the nitrogen atom to which they are attached, form a heterocyclyl group.

The term “alkylthio” as used herein refers to a sulfur atom connected to an alkyl, alkenyl, or alkynyl group as defined herein.

The term “arylthio” as used herein refers to a sulfur atom connected to an aryl group as defined herein.

The term “alkylsulfonyl” as used herein refers to a sulfonyl group connected to an alkyl, alkenyl, or alkynyl group as defined herein.

The term “alkylsulfinyl” as used herein refers to a sulfinyl group connected to an alkyl, alkenyl, or alkynyl group as defined herein.

The term “dialkylaminosulfonyl” as used herein refers to a sulfonyl group connected to a nitrogen further connected to two alkyl groups, as defined herein, and which can optionally be linked together to form a ring with the nitrogen. This term also includes the group where the nitrogen is further connected to one or two alkenyl groups in place of the alkyl groups.

The term “dialkylamino” as used herein refers to an amino group connected to two alkyl groups, as defined herein, and which can optionally be linked together to form a ring with the nitrogen. This term also includes the group where the nitrogen is further connected to one or two alkenyl groups in place of the alkyl groups.

The term “dialkylamido” as used herein refers to an amido group connected to two alkyl groups, as defined herein, and which can optionally be linked together to form a ring with the nitrogen. This term also includes the group where the nitrogen is further connected to one or two alkenyl groups in place of the alkyl groups.

The term “substituted” as used herein refers to a group that is substituted with one or more groups including, but not limited to, the following groups: halogen (e.g., F, Cl, Br, and I), R, OR, ROH (e.g., CH₂OH), OC(O)N(R)₂ (also known as carbamate), CN, NO, NO₂, ONO₂, azido, CF₃, OCF₃, methylenedioxy, ethylenedioxy, (C₃-C₂₀)heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen, (C₁-C₂₀)alkyl, (C₆-C₂₀)aryl, heterocyclyl or polyalkylene oxide groups, such as polyalkylene oxide groups of the formula —(CH₂CH₂O)_{f}—R—OR, —(CH₂CH₂CH₂O)_{g}—R—OR, —(CH₂CH₂O)_{f}(CH₂CH₂CH₂O)_{g}—R—OR each of which can, in turn, be substituted or unsubstituted and wherein f and g are each independently an integer from 1 to 50 (e.g., 1 to 10, 1 to 5, 1 to 3 or 2 to 5). Substituted also includes a group that is substituted with one or more groups including, but not limited to, the following groups: fluoro, chloro, bromo, iodo, amino, amido, alkyl, hydroxy, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocycyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

In some instances, the compounds described herein (e.g., compounds of the formulae (I), (Ia)-(Ik), (II), (III), (IV), (V), and (VI)) can contain chiral centers. All diastereomers of the compounds described herein are contemplated herein, as well as racemates.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quatemary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

As used herein, the term “subject” or “patient” refers to any organism to which a composition described herein can be administered, e.g., for experimental, diagnostic, prophylactic and/or therapeutic purposes. Subject refers to a mammal receiving the compositions disclosed herein or subject to disclosed methods. It is understood and herein contemplated that “mammal” includes but is not limited to humans, non-human primates, cows, horses, dogs, cats, mice, rats, rabbits, and guinea pigs.

Each embodiment described above is envisaged to be applicable in each combination with other embodiments described herein. For example, embodiments corresponding to formula (I) are equally envisaged as being applicable to formulae (Ia)-(Ik). Likewise, embodiments that are corresponding to formula (II) are equally envisaged as being applicable to formulae (III)-(VI). Likewise, embodiments that are corresponding to formula (III) are equally envisaged as being applicable to formulae (I), (II), and (IV)-(VI) and so on.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed can be resorted to by those of ordinary skill in the art, and that such modifications and variations are considered to be within the scope of embodiments of the present disclosure

The invention is now described with reference to the following Examples. The following working examples therefore, are provided for the purpose of illustration only and specifically point out certain embodiments of the present invention, and are not to be construed as limiting in any way the remainder of the disclosure. Therefore, the examples should be construed to encompass any and all variations which become evident as a result of the teaching provided herein.

### EXAMPLES

The present disclosure can be better understood by reference to the following examples which are offered by way of illustration. The disclosure is not limited to the examples given herein.

### INTRODUCTION

The human proteasome is part of the cellular machinery that regulates protein degradation. Most proteins are degraded by the 26S proteasome via a ubiquitin-dependent mechanism, however intrinsically disordered proteins (unstructured) proteins can also be degraded the 20S isoform of the proteasome via a ubiquitin-independent mechanism. Intrinsically disordered proteins (IDPs) are named for their lack of tertiary structure allowing them to adopt numerous conformations and interact with multiple binding partners. When the synthesis of IDPs outpaces their rate of degradation, they accumulate and induce toxic signaling events that drive many human diseases.

Arguably the most infamous IDP associated with cancer initiation, progression and relapse is the pro-oncogenic transcription factor, c-MYC. Over-expression of c-MYC is the driving force in an astonishing 60-70% of all human cancers including multiple myeloma, histiocytic sarcoma, myeloid leukemia, glioblastoma, melanoma, breast cancer, colon cancer, cervical cancer, small-cell lung carcinoma, and osteosarcoma. Small molecule 20S proteasome activators can reduce c-MYC protein levels and therefore prevent the initiation progression and relapse in c-MYC driven cancers.

The disclosure relates to small molecule 20S proteasome activators of the formulae (I), (Ia)-(Ik), (II), (III), (IV), and (IV) as therapeutic agents to treat amyloidogenic diseases including neurodegenerative diseases and type II diabetes. Neurodegenerative diseases include: Alzheimer's disease (AD) and other dementias, Parkinson's disease (PD) and PD-related disorders, Prion disease, Motor neuron diseases (MND), Huntington's disease (HD), Spinocerebellar ataxia (SCA) and Spinal muscular atrophy (SMA). Overwhelming evidence points towards the accumulation and subsequent oligomerization of intrinsically disordered proteins (IDPs) such as amyloid-b, a-synuclein, polyQ, and dipeptide repeat (DPR) units as the driving causes of these diseases. These soluble oligomeric forms are also responsible for impairing proteasome function, which further drives disease progression. Robust data demonstrates that enhancing proteasome activity prevents the accumulation of IDPs, reduce brain damage, prevent dementia and may be a new therapeutic strategy to treat neurodegenerative diseases.

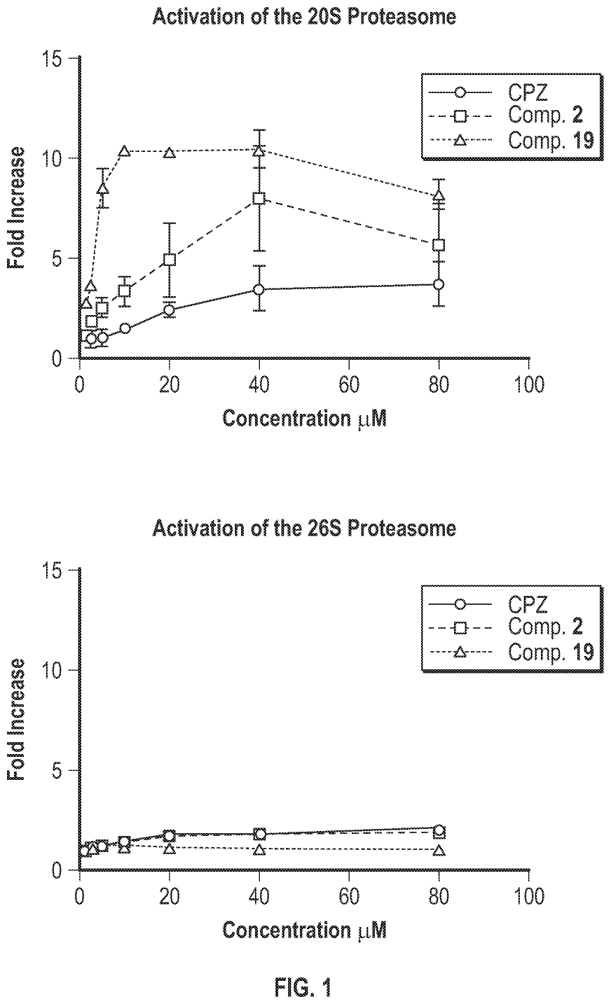

Identification: The small molecule antipsychotic drug chlorpromazine was identified as a promising new scaffold for the development of 20S activators due to its strong enhancement of 20S proteolysis. To assess chlorpromazines 20S proteasome activity, a series of assays were performed using each of three fluorogenic peptide substrates. These substrates were a chymotryptic-like (CT-L), a trypsin-like (T-L) and a caspase-like (Casp-L) substrate, one for each of the catalytic sites of the proteasome. It has been shown that the proteasome's active sites allosterically regulate each other in the presence of their individual substrates. Therefore, a combination of the three probes to represent the overall activity of a 20S activator more accurately in a system in which all catalytic sites are interacting. Chlorpromazine activates all the CT-L sites of the 20S proteasome (FIG. 1) and achieved a doubling of activity (hereafter referred to as AC₂₀₀) using the combination of probes at 13.5 μM (i.e. AC₂₀₀ 13.5 mM), with a maximum fold enhancement of nearly 4-fold (i.e. 400%). Moreover, chlorpromazine did not enhance the proteolytic activity of the 26S proteasome.

Design of analogues: The neuroleptic drugs chlorpromazine was identified as hits in our reported study. While not being bound by any specific theory, their clinical neuroleptic activities are largely attributed to their interactions with the dopamine 2 receptor (D2R), which requires protonation of their basic nitrogen. Therefore, the interaction with the D2R can be abrogated by elimination of this nitrogen or by using less basic amines, such as amides or anilines in this position. The approach of modifying the basicity of this critical nitrogen was shown to be successful in abrogated the D2R binding, while improving 20S proteasome activity in our publication on this work in ACS Chem. Biol. 2017, 12 (9), 2240-2247, which is incorporated by reference as if fully set forth herein.

Synthesis. To increase the potency and efficiency of our lead compound 2, different substituents on the benzyl group and pendant groups were prepared and evaluated for enhancement of 20S proteasome activity (Scheme 1).

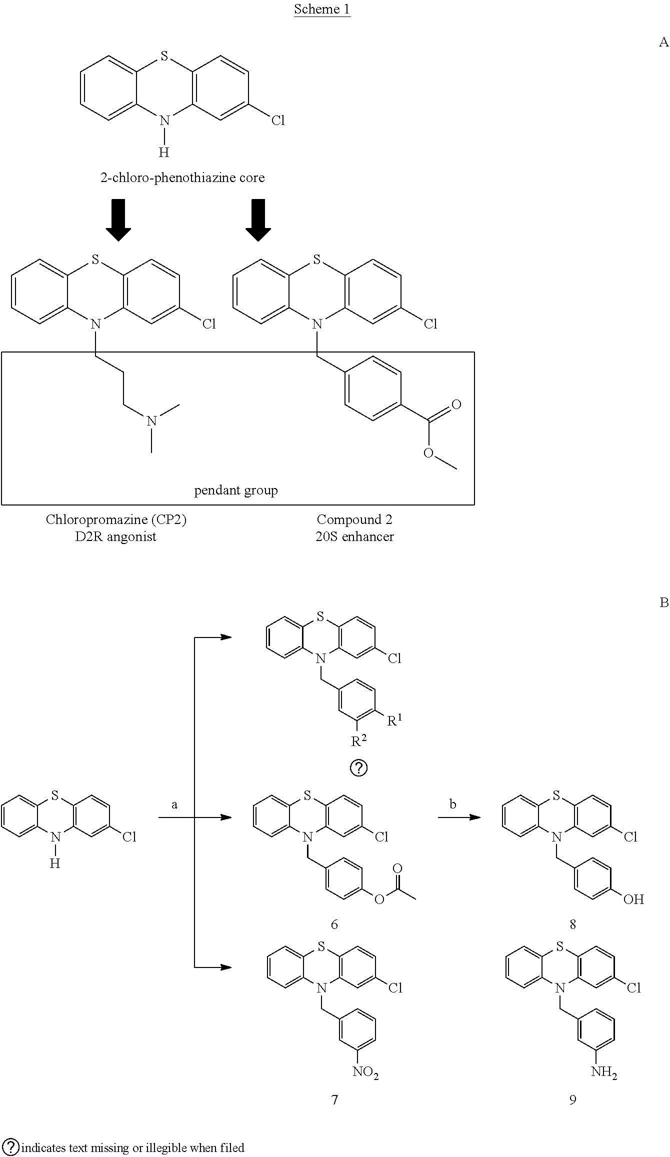

In Scheme 1, panel A, substitution of the 2-chloro-phenothiazine core with different pendant groups to gain diverse target selectivity. In Scheme 1, panel B, the synthesis of compounds 1-9. (a) NaH, THF or DMF, rt for 30 min to 1 h, then the addition of alkyl halide, rt. 16 h to obtain compound 1 (17%), compound 2 (92%), 3 (7.6%), 4 (28%/), 5 (20%), 6 (12%), and 7 (31%) (b) KOH, water/ethanol 1:1, reflux 4 h for compound 8 (21%) (c) SnCl2, EtOAc, reflux for 2 h to obtain compound 9 in quantitative yields.

As a control, a non-substituted benzyl group (1) was synthesized by treating 2-chlorophenothiazine with NaH followed by (bromomethyl)-benzene (Scheme 1). This general procedure of deprotonating 2-chlorophenothiazine and then addition of a halogenated benzyl group was used to prepare compounds 2-7 (Scheme 1). The role of the aryl ester moiety in activating the 20S proteasome was explored by substituting the ester with a carboxylate or an alcohol. The carboxylate derivative (6) was prepared by treating 2-chloro-phenothiazine with NaH and 4-(bromomethyl)phenyl acetate. Compound 6 was then treated with KOH to produce the alcohol derivative (8). Next, the impact of the size of the substituent was investigated by exchanging the methyl ester with a tert-butyl ester to provide compound 3. To examine the effect of the position, three meta substituted benzyl derivatives were synthesized, the meta-substituted benzyl ether (4), benzyl ester (5), and benzyl amine (9). The original parent compound, CPZ, contains a dimethyl amine group attached to the phenothiazine core by a 3-carbon linker. Compound 9 was designed to incorporate this amine group (albeit less basic), while maintaining the benzyl moiety through the conversion of a nitro group to an amine. The nitro-benzyl (7) group was added to the 2-chlorophenothiazine group using the general procedure previously described. The nitro moiety on the aryl group was then reduced using SnCl₂ to generate compound 9 in quantitative yields (Scheme 1).

Next, the scope of the pendant motifs on the phenothiazine core were investigated. New pendant moieties were designed to mimic the dimethyl amine group of CPZ. The clinical neuroleptic activity of CPZ is largely attributed to its interaction with the dopamine 2 receptor (D2R), which requires protonation of the dimethyl amine group of CPZ.⁶⁷⁻⁶⁸ Therefore, the interaction with the D2R can be abrogated using less basic amines, such as amides or anilines in this position. In addition to altering the basicity, the length of the methylene linker was modified to investigate the effect of flexibility and distance on 20S proteasome activation. Amide 11 was prepared via the nitrile intermediate (10) obtained by treating 2-chlorophenothazine with 5-bromovaleronitrile. The nitrile group was then converted to the amide in a 95% yield using an H₂SO₄ catalyzed hydrolysis (Scheme 2).

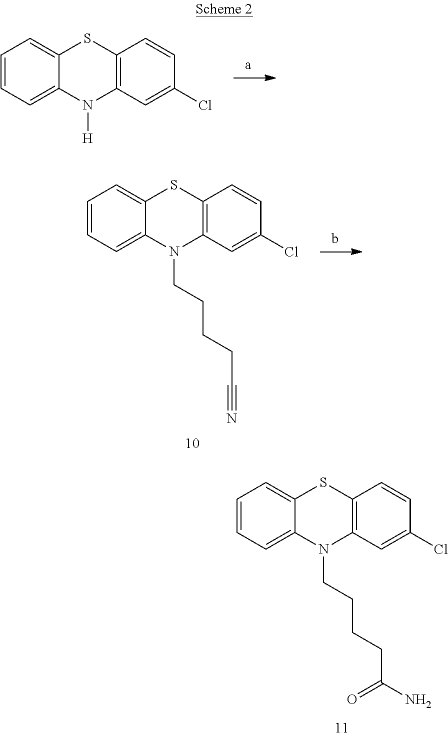 [SMILES: Clc1ccc2c(c1)Nc1ccccc1S2 ; N#CCCCCN1c2ccccc2Sc2ccc(Cl)cc21 ; NC(=O)CCCCN1c2ccccc2Sc2ccc(Cl)cc21]

Scheme 2 shows the synthesis of compounds 10 and 11. (a) NaH, THF, rt 30 min, then 5-bromovaleronitrile, rt 48 h to yield compound 10 (88%) (b) H2SO4, rt 2.5 h to yield compound 11 (95%).

Amide 15 was then synthesized to compare the four-methylene linker to the two-methylene chain seen in CPZ. While compounds 11 and 15 were made to investigate the role of nitrogen on an aliphatic chain, both compounds have a primary nitrogen. The amide was further modified to include additional substituents. Compound 16 was designed to have a similar pendant to the parent compound CPZ, but with the dimethyl amine transformed to a dimethyl amide.

Based on the activity displayed by compound 2, other pendant groups containing an aromatic group were explored. An unsubstituted aniline was reacted with acryloyl chloride and then treated with 2-chlorophenothiazine in the presence of Triton B, producing compound 17 (60%). Then, the installment of a fluoro-substituent on the aniline was explored and the fluoroaniline derivative (18) was synthesized (Scheme 3).

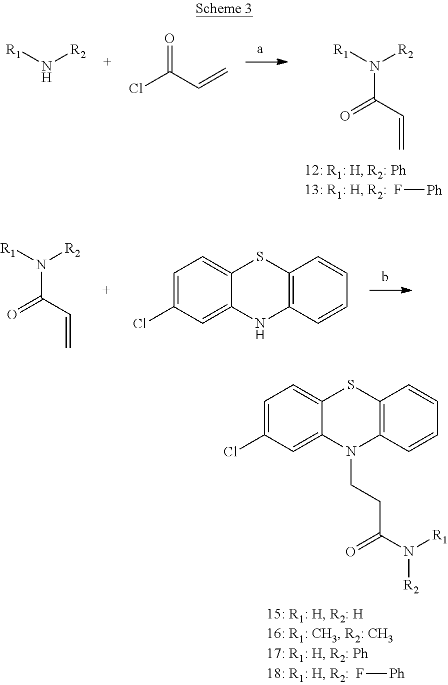

Scheme 3 shows the synthesis of compounds 15-18. (a) TEA, DCM, rt for 12 h (b) Triton-B and 2-chlorophenothiazine in toluene, rt to reflux 24 h, to yield compounds 15 (25%), 16 (85%), 17 (60%), and 18 (30%).

Finally, compound 19 was designed to mimic the di-substituted amine group of CPZ, but with an electron depleted nitrogen to prevent N-protonation. The bis-fluorodiphenyl amine group was synthesized using a Chan-Lam-Evans like coupling that was previously reported.⁷¹ The bis-fluorodiphenyl amine was transformed to acrylamide 14 by treating it with acryloyl chloride and TEA in DCM and was refluxed overnight. This acryl amide was coupled to the 2-chlorophenothaizine core using Triton-B in toluene and then refluxed for 24 hours, resulting in compound 19 (50%) (Scheme 4).

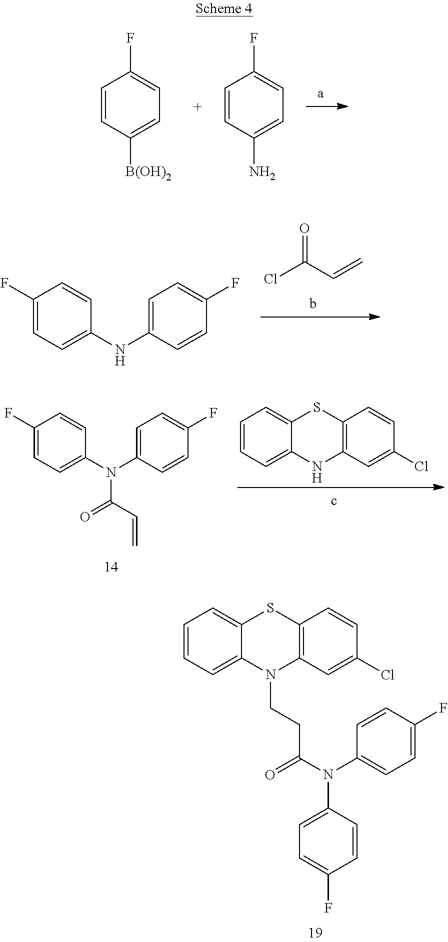

Scheme 4 shows the synthesis of compound 19. (a) Cu(OAc)2, TEA, air, DCM, 4 Å molecular sieves, rt 16 h (48%) (b) TEA in DCM, reflux for 12 h, 60% (c) Triton-B and 2-chlorophenothiazine in toluene, rt to reflux 24 h, 19 (50%).

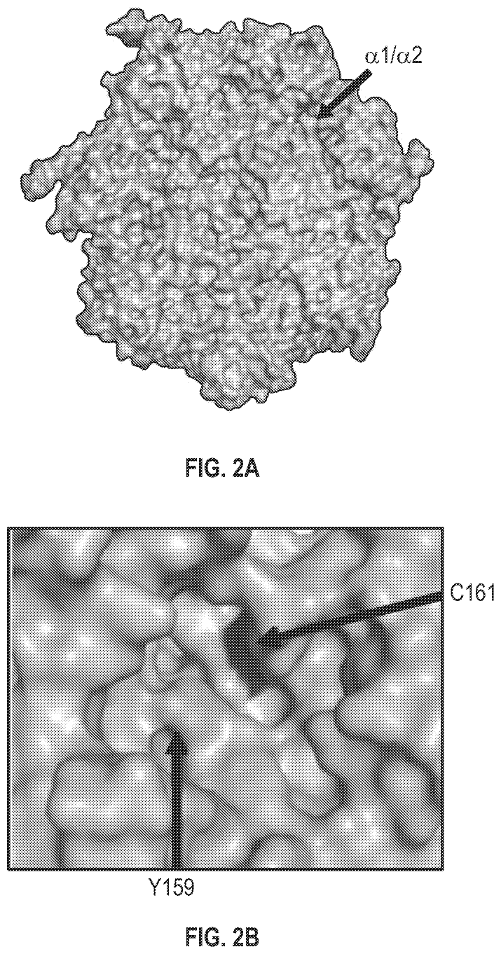

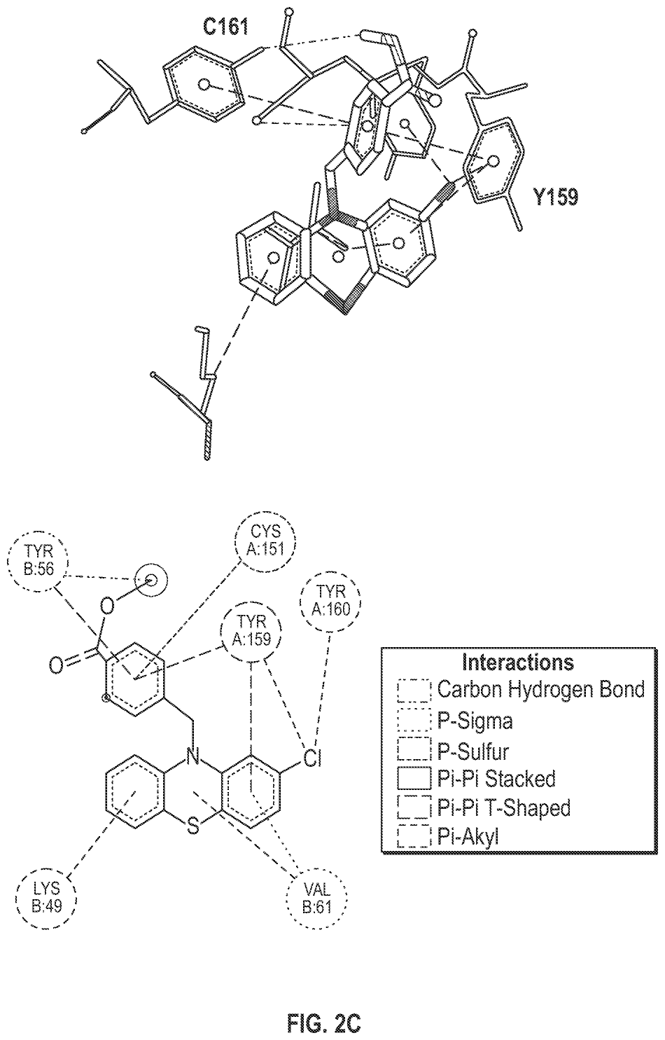

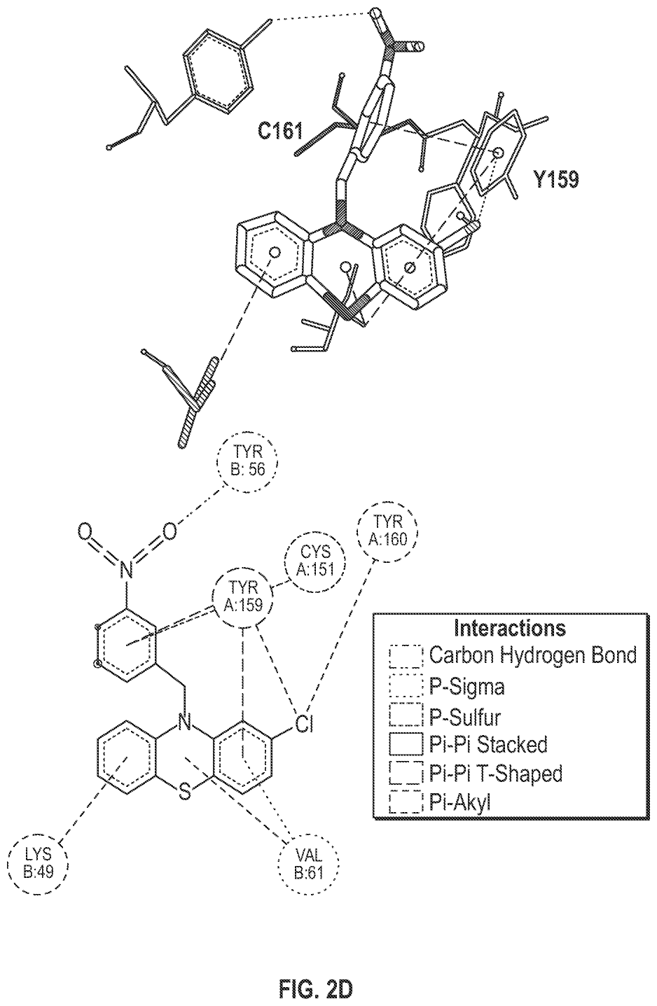

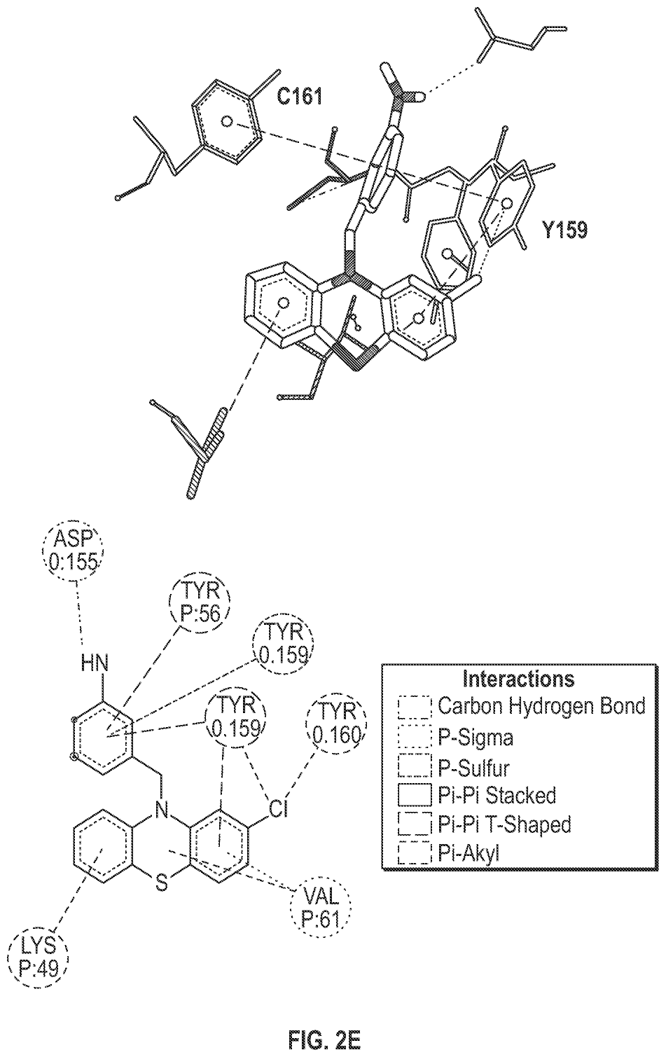

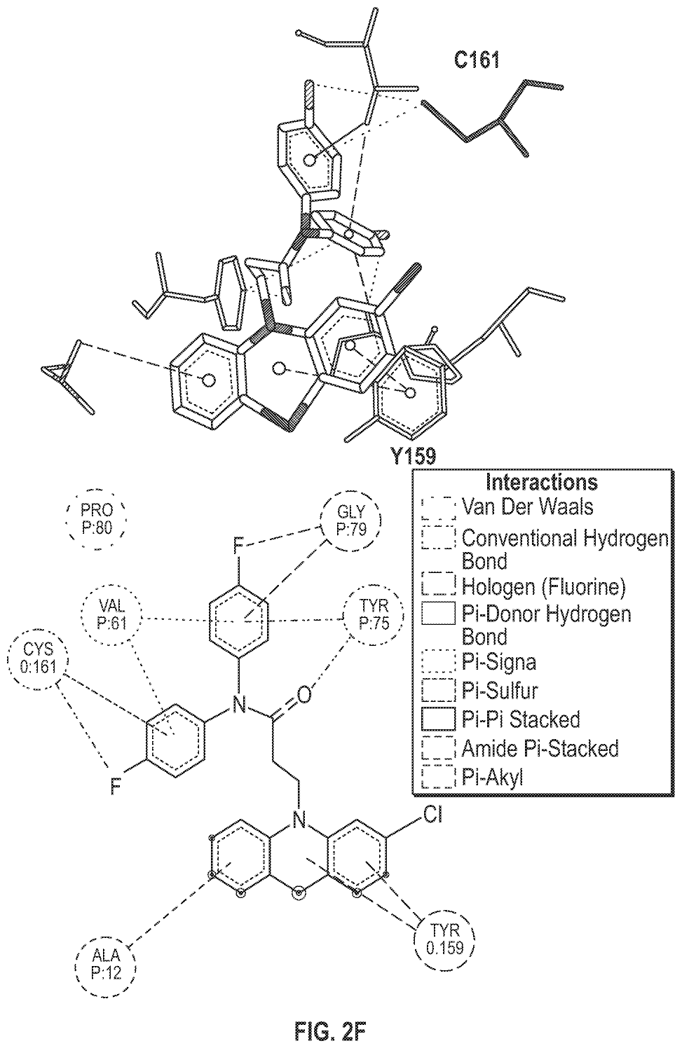

Docking studies: Molecular docking studies are helpful in understanding the interaction of our compounds with the 20S proteasome. Herein, unbiased molecular docking has been extended to propose key interactions between the reported compounds and amino acid residues within the α ½ pocket. Prior docking studies indicate that the CPZ analogue, compound 2, primarily binds in the α ½ pocket and possibly interacts with several amino acid residues (FIG. 2A). We hypothesized that a single amino acid acts as an anchor point for these small molecules. Now with the addition of the compounds reported herein, a more extensive molecular docking analysis is possible, allowing for the comparison of common amino acid interactions. To perform these studies, we followed the same procedure we utilized previously, but used Discovery Visualizer to further investigate individual interactions with the amino acids in the α ½ pocket. An analysis of the interactions between the amino acids and the active compounds revealed the dominancy of C161 and Y159. (FIG. 2B). Each of the active compounds had some type of interaction with these amino acids, however, not all the interactions were of the same type. Y159 interacts with the active compounds through both parallel π-π stacking and T-shaped π-π stacking, while the inactive compounds 6 only interacts through T-shaped π-π stacking, indicating that parallel π-π stacking may be a required interaction. Furthermore, considering the potency difference between compounds 7 and 9, this interaction may have some “aromatic donor-acceptor” characteristics. The electron rich benzyl derivative, 9, is 3× less potent than its electron poor counterpart, 7. It has been reported that this “face-centered” stacking is often favored during this aromatic donor-acceptor interaction and the electron depletion impacts the strength of this stacking, the more electron deficient arenes stack very effectively to the electron-rich donor.⁷² These findings strongly suggests that this π-π stacking is a key interaction between the small molecules and the α ½ pocket and requires further exploration.

C161 interacts with the molecules through either sulfur-π or sulfur-alkyl interactions. Compounds 1, 4, 5, 17, and 18 interact with C161 through an edge-on sulfur-alkyl interaction. The weak sulfur-alkyl interaction could cause the lowered activity of these molecules. Compounds 2, 7, 9, and 19, are the most potent and efficient small molecules reported, and are predicted to interact with C161 through an edge-on π-sulfur interactions (FIGS. 2C-F). In literature, ab-initio calculations have been used to determine the ideal geometry for cysteine-pi interactions, identifying abundance of strong edge-on cysteine-π interactions. These findings lead us to believe that C161 may be paramount for 20S proteasome activation.

In vitro testing, 20S activation: The 20S proteasome activity of the compounds was assessed by utilizing the standard fluorogenic 7-amino-methylcoumarin (AMC) conjugated small peptide substrates that correspond to each of the catalytic sites in the 20S proteasome: chymotryptic-like site (Suc-LLVY-AMC), caspase-like site (Z-LLE-AMC), and trypsin-like site (Boc-LRR-AMC). When the small peptide substrate is degraded by the 20S proteasome, the AMC fluorophore is released and quantified to measure the rate of proteolytic cleavage. The 20S proteasome activity of the small molecules was evaluated at varying concentrations to determine the concentration at which the rate of proteolysis was doubled (i.e. the concentration needed to induce 200% activity) compared to the vehicle control (hereafter named as AC₂₀₀). Concentrations ranging from 1.25 μM to 80 μM of each of compound were added to wells that contained 0.5 nM of purified human proteasome.

The overall enhancement of the rate of proteolysis of the 20S proteasome by a small molecule was first evaluated by adding a combination of all three peptide substrates to the treated sample. The combination of all three substrates represents a more accurate measurement of proteolytic activity of the proteasome because the catalytic sites housed in the β ring of the 20S proteasome communicate through allosteric interactions. For example, when a protein substrate encounters the chymotryptic-like site, a conformational change will occur activating the caspase-like site.⁶¹ The individual contribution of each of the three catalytic sites was subsequently evaluated by adding only the corresponding peptide probes to gain insight into the contribution of each site on the overall enhanced proteolytic activity.

The fluorescent output, due to release of the AMC fluorophore, was then converted to the fold increase compared to a vehicle control sample, and the AC₂₀₀ was calculated. To rigorously eliminate false positives due to minor changes in background activity (0-20%), compounds that exhibit a maximum fold enhancement of <2 fold (i.e. <200%) at a concentration >20 μM were considered inactive. (Table 1).

TABLE 1

| | | CT-L/Casp-L/T-L | | Casp-L site | | T-L site | | CT-L site | |
|---|---|---|---|---|---|---|---|---|---|
| 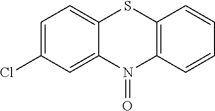 | | AC₂₀₀ | Max Fold | AC₂₀₀ | Max Fold | AC₂₀₀ | Max Fold | AC₂₀₀ | Max Fold |
| CPZ | 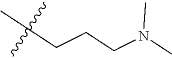 [SMILES: *CCCN(C)C] | 13.5 μM | 4 | >80 μM | — | >80 μM | — | 7 μM | 8 |
| 1 | 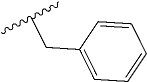 [SMILES: *Cc1ccccc1] | 1.5 μM | 2 | >80 μM | — | 3.1 μM | 2 | 2.8 μM | 3 |
| 2 | 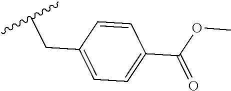 [SMILES: *Cc1ccc(C(=O)OC)cc1] | 1.4 μM | 9 | 2.7 μM | 7 | 2.6 μM | 15 | 1.9 μM | 12 |
| 3 | 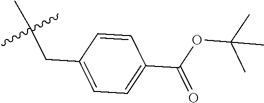 [SMILES: *Cc1ccc(C(=O)OC(C)(C)C)cc1] | >80 μM | — | >80 μM | — | >80 μM | — | >80 μM | — |
| 4 | 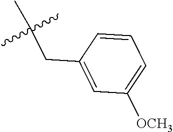 [SMILES: *Cc1cccc(OC)c1] | 1.5 μM | 4 | 1.3 μM | 4 | 5.4 μM | 2 | >80 μM | — |
| 5 | 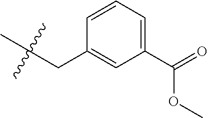 [SMILES: *Cc1cccc(C(=O)OC)c1] | 2.4 μM | 6 | 9.7 μM | 4 | 1.9 μM | 8 | 2.4 μM | 6 |
| 6 | 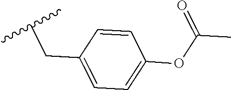 [SMILES: *Cc1ccc(OC(C)=O)cc1] | >80 μM | — | >80 μM | — | >80 μM | — | >80 μM | — |
| 7 | 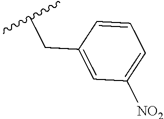 [SMILES: *Cc1cccc([N+](=O)[O-])c1] | 1.1 μM | 8 | 1.2 μM | 12 | 1.6 μM | 8 | 1.1 μM | 9 |
| 8 | 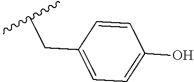 [SMILES: *Cc1ccc(O)cc1] | >80 μM | — | >80 μM | — | >80 μM | — | >80 μM | — |
| 9 | 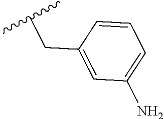 [SMILES: *Cc1cccc(N)c1] | 3.6 μM | 8 | 3.8 μM | 10 | 9.2 μM | 10 | 6.5 μM | 9 |
| 10 | 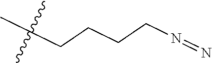 [SMILES: *CCCCN=N] | >80 μM | — | >80 μM | — | >80 μM | — | >80 μM | — |
| 11 | 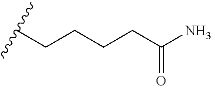 [SMILES: *CCCCC(N)=O] | >80 μM | — | >80 μM | — | >80 μM | — | >80 μM | — |
| 15 | 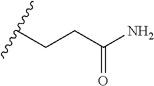 [SMILES: *CCC(N)=O] | >80 μM | — | >80 μM | — | >80 μM | — | >80 μM | — |
| 16 | 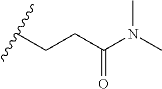 [SMILES: *CCC(=O)N(C)C] | >80 μM | — | >80 μM | — | >80 μM | — | >80 μM | — |
| 17 | 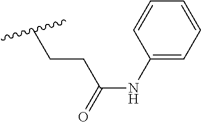 [SMILES: *CCC(=O)Nc1ccccc1] | 40 μM | 2.5 | >80 μM | — | 80 μM | 2 | >80 μM | — |
| 18 | 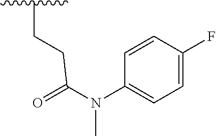 [SMILES: *CCC(=O)N(C)c1ccc(F)cc1] | | | | | | | | |
| 19 | 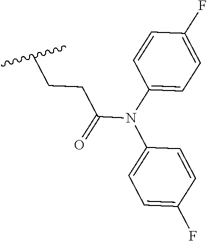 [SMILES: *CCC(=O)N(c1ccc(F)cc1)c1ccc(F)cc1] | 0.4 μM | 11 | 1.8 μM | 9.4 | 0.3 μM | 6 | 0.7 μM | 9.4 |

Table 1 shows increase in proteolytic degradation of Suc-LLVY-AMC chymo-tryptic-like probe (CT-L), Z-LLE-AMC caspase-like probe (Casp-L), and Boc-LRR-AMC trypsin-like probe (T-L), combined and each of the individual three substrate probes, by purified human 20S proteasome treated with compounds 1-11 and 15-19. AC₂₀₀ represents the concentration needed to double (e.g., 200%) the rate of substrate degradation compared to vehicle control. Max fold is the maximum fold rate increase compared to vehicle control.

Several derivatives of CPZ significantly enhanced 20S mediated proteolysis. The unsubstituted benzyl derivative (1) enhanced 20S proteasome mediated proteolysis 2-fold (i.e. 200% increase over vehicle control) at a relatively low AC₂₀₀ of 1.5 μM. Compound 1's overall rate of proteolysis appeared to be due to the enhanced activity at the chymotryptic-like and trypsin-like sites. The t-butyl (3), the acetate (6), and alcohol (8) benzyl derivates are all deemed inactive as they do not reach a 200% increase in proteasome activity at the concentrations tested. Analog 4 has an AC₂₀₀ of 1.5 μM, which is very similar to compound 2 (1.4 μM). However, compound 4 is less than half as efficient as compound 2 (maximum fold of 4). This finding suggests that either the carbonyl group aids activation and/or it may impact the interactions of the benzyl ring. Interestingly, the meta derivative of compound 2 (5), has a higher AC₂₀₀ (2.4 μM) but a lower maximum fold increase of 6 (i.e. 600% increase over vehicle control). In contrast, compound 7 had a similar maximum fold increase of 8 (i.e. 800% increase over vehicle control) and had a relatively low AC₂₀₀ (1.1 μM). The transformation of this nitro group into the primary amine does not affect the overall 20S proteasome activation but did raise the AC₂₀₀ (3.6 μM).

Compounds 10, 11, 15, and 16 were all inactive, and compounds 17 and 18 show low activation (maximum 2.5 and 3-fold increase over vehicle control, respectively). The addition of two aryl groups onto the nitrogen enhances 20S proteasome activation. Compound 19 displays a maximum 11-fold (i.e. 1100% increase over vehicle control) enhancement of the proteolytic activity of the 20S proteasome, with potent AC₂₀₀ values and fold enhancements of each of the three catalytic sites. Compound 19 activated the trypsin-like site with an AC₂₀₀ of 0.3 μM, (6-fold maximum enhancement), the chymotryptic-like site with an AC₂₀₀ of 0.7 μM, (9.4-fold maximum enhancement), and the caspase-like site with an AC₂₀₀ of 1.8 μM, (9.4-fold maximum enhancement).

Before evaluating the ability of compound 19's proteolytic activity towards pathogenically relevant IDPs, the selectivity of compound 19 towards the 20S proteasome over the 26S proteasome was evaluated. The fluorogenic small peptide degradation assay was conducted again, with the 26S proteasome in place of the 20S proteasome. To ensure that the 26S proteasome remained in its fully assembled complex, ATP and magnesium chloride were added. CPZ and compound 2 showed a slight activation of the 26S proteasome at higher concentrations (maximum 2-fold increase over the vehicle control), while compound 19 did not display any 26S proteasome activation.

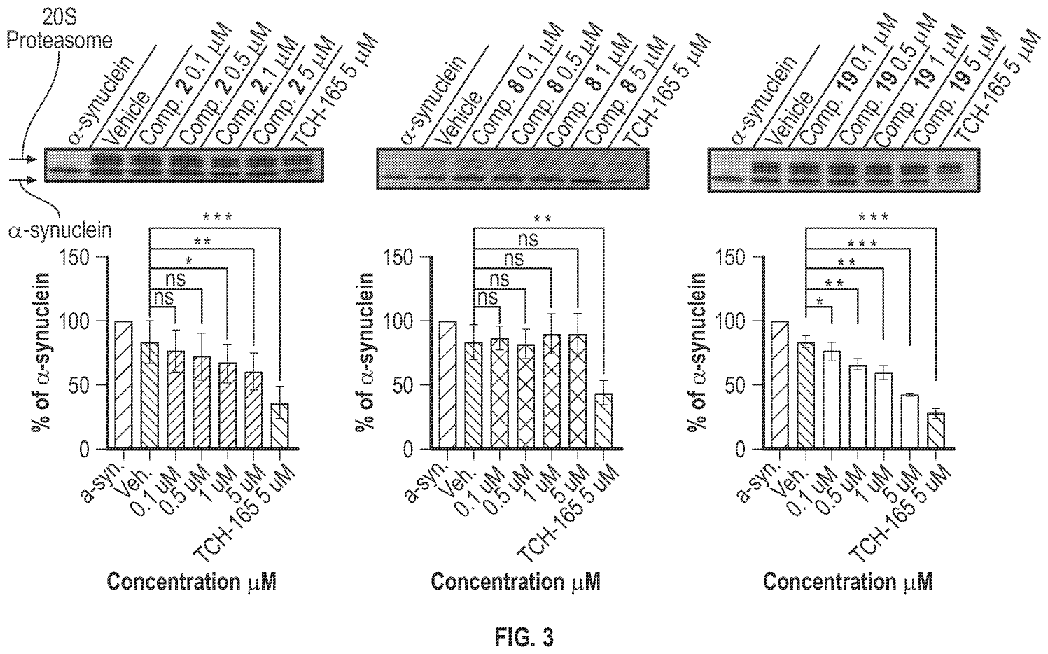

In vitro testing, IDP degradation: The ability of our small molecules 20S proteasome activators to degrade pathologically relevant IDPs was evaluated in vitro and in cells. The efficacy of compound 19 on 20S-mediated degradation of α-synuclein was evaluated in vitro using compound 2 and TCH-165, a previously reported 20S proteasome enhancer, as a positive control and the inactive analogue (8) as a negative control. Purified 20S proteasome was incubated at 37° C. with either compound 2, 8, 19 (concentrations 0.1 μM to 5 μM) or TCH-165 (5 μM) for 15 minutes. Then, purified α-synuclein was added and the digestion was incubated at 37° for 6 hours. An SDS loading buffer was added and the samples were boiled to halt α-synuclein degradation. The percentage of remaining α-synuclein was visualized and quantified using silver staining (FIG. 3). Compound 2 was able to increase the rate of degradation of full length α-synuclein. At the highest concentration (5 μM), α-synuclein was reduced up to 33%. While there was not a significant decrease in the lower concentrations of compound 2, at 0.5 μM of compound 19, a 34% decrease of α-synuclein was observed. Compound 19 had a clear dose dependent depletion of α-synuclein, with a 57% reduction at 5 μM.

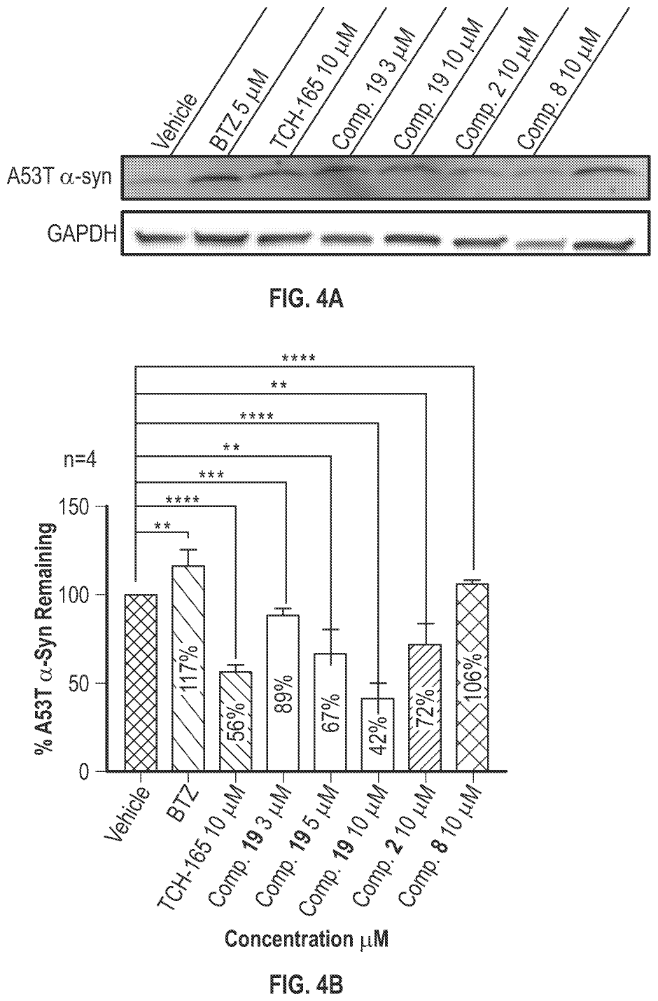

Cellular efficacy: The A53T mutation of α-synuclein has been linked to early on-set familial Parkinson's disease and appears to oligomerize faster than the wild-type protein. We evaluated compound 19's ability to prevent the accumulation of the A53T mutant α-synuclein in cells. Hek-293T cells were transiently transfected with an inducible Tet-One A53T mutant α-synuclein. The cells were treated with 100 ng/mL of doxycycline to induce the production of the A53T mutant. After 24 hours, the cells were treated with compound 19 (3 μM, 5 μM, and 10 μM), the control compounds TCH-165, compound 2, compound 8 (10 μM each), and proteasome inhibitor bortezomib (BTZ, 5 μM), for 10 hours. The cells were then lysed and the remaining A53T α-synuclein was visualized and quantified via Western Blotting (FIG. 4, n=4). Bortezomib (BTZ), a known proteasome inhibitor, was used a negative control and TCH-165 was once again used as a positive control and was observed to decrease the concentration of accumulated A53T α-synuclein by 40%. Compound 2 displayed a reduction of 28% of the A53T mutant α-synuclein, while compound 8 had no decrease in the A53T mutant. Compound 19 was able to reduce the concentration of the A53T mutant α-synuclein significantly at its lowest concentration, 3 μM and a dose dependent enhanced degradation was observed. At the highest concentration of compounds 19, 10 μM, the A53T α-synuclein was decreased by 58%. The potency and efficiency displayed by compound 19 towards the 20S proteasome in previous experiments does translate to the degradation of A53T mutant α-synuclein in HEK293T cells.

### Synthetic Studies:

General Experimental Information. Reactions were carried out under a nitrogen atmosphere in flame-dried glassware. Solvents and reagents were purchased from commercial suppliers and used without further purification. Anhydrous THF was distilled over sodium and benzophenone directly before use. Magnetic stirring was used for all reactions. Yields refer to chromatographically and spectroscopically pure compounds unless otherwise noted. Infrared spectra were recorded on a Jasco Series 6600 FTIR spectrometer. ¹H and ¹³C NMR spectra were recorded on a Varian Unity Plus-500 or 600 spectrometers. Chemical shifts are reported relative to the residue peaks of the solvent (CDCl3: 7.26 ppm for 1H and 77.0 ppm for 13C) (DMSO-d6: 2.50 ppm for 1H and 39.5 ppm for 13C). The following abbreviations are used to denote the multiplicities: s=singlet, d=doublet, dd=doublet of doublets, t=triplet, and m=multiplet. HRMS were obtained at the Mass Spectrometry Facility of Michigan State University with a Micromass Q-ToF Ultima API LC-MS/MS mass spectrometer.

General benzylation procedure: 2-chlorophenothiazine (1 eq) was dissolved in anhydrous 3-5 mL of DMF at room temperature under an inert atmosphere in a round bottom flask. Sodium hydride (1.1 eq) was added as a single portion, vigorous bubbling is observed, and the mixture allowed to stir at room temperature for 0.5 h wrapped in foil. Substituted benzyl derivate (1.5 eq) is added as a single portion and allowed to react at room temperature in the dark (i.e. wrapped in foil) for 16 h. After 16 h, the reaction is diluted with ether (ca. 2 solvent volumes) and poured into separatory funnel containing a 10% wt/wt solution of LiBr in DI water. The ether layer is carefully washed 2× with LiBr (aq) and then Brine (1× solvent volume), dried over sodium sulfate and concentrated under reduced pressure. Crude material was then purified using an automated CombiFlash chromatograph (silica gel, 20-40 μm, gradient 30% ethyl acetate in hexane).

General acylation procedure: The desired amine (1 eq) and TEA (1.33 eq) in DCM were cooled to 0° C. and acryloyl chloride (1.2 eq) was added drop wise. The reaction mixture was warmed to warm temperature and left to stir for 12 hours. The crude reaction was washed with 2×10 mL of Brine and extracted with 2×10 mL of DCM. The organic layer was dried over sodium sulfate, filtered, and concentrated under reduced pressure. The desire product was purified using an automated CombiFlash chromatograph (silica gel, 20-40 microns, gradient 40% ethyl acetate and 60% hexane).

General Michael-Addition procedure: 2-chloro-phenothiazine (2 eq) and the acyl amine (1 eq) were dissolved in toluene, then Triton B (0.5 eq) was added drop wise and the solution was refluxed for 24 hours. The reaction mixture was cooled to room temperature and washed with 2×10 mL of NaOH (aq) and extracted with 2×10 mL of EtOAc. The organic layer was dried over sodium sulfate and filtered. The crude mixture was concentrated under reduced pressure and purified using an automated CombiFlash chromatograph (silica gel, 20-40 microns, gradient 20% ethyl acetate and 80% hexane).

### Example 1

10-Benzyl-2-chloro-10H-phenothiazine (1). General benzylation procedure provided a colorless oil product (0.200 g, 17%). IR: 3060 cm⁻¹, 2924 cm⁻¹. ¹H NMR (CDCl₃, 500 MHz) δ 7.38-7.34 (m, 2H), 7.34-7.30 (m, 2H), 7.30-7.28 (m, 1H), 7.12 (dd, J=7.6, 1.6 Hz, 1H), 7.05-6.97 (m, 2H), 6.91 (td, J=7.5, 1.2 Hz, 1H), 6.87 (dd, J=8.2, 2.0 Hz, 1H), 6.68 (d, J=1.2 Hz, 1H), 6.66 (dd, J=2.7, 1.6 Hz, 1H), 5.06 (s, 2H). ¹³C{¹H} NMR (CDCl₃, 126 MHz) δ 145.8, 143.8, 136.0, 133.2, 128.9, 127.5, 127.4, 127.3, 127.0, 126.6, 123.1, 123.0, 122.4, 121.9, 115.9, 115.7, 52.7. HRMS (ESI+) m/z: [(M+H)+] calc'd for (C₁₉H₁₅ClNS⁺) 324.05690; Found 324.0611.

### Example 2

Tert-butyl 4-((2-chloro-10H-phenothiazin-10-yl)methyl)benzoate (3). General benzylation procedure provided a white solid (0.064 g, 7.6%). IR: 3100 cm⁻¹, 2949 cm⁻¹, 1713 cm⁻¹. ¹H NMR (CDCl₃, 500 MHz) δ 7.98 (d, J=8.2 Hz, 2H), 7.36 (d, J=8.1 Hz, 2H), 7.09 (dd, J=7.5, 1.5 Hz, 1H), 6.99 (d, J=8.1 Hz, 1H), 6.97 (dd, J=7.9, 1.6 Hz, 1H), 6.89 (td, J=7.5, 1.2 Hz, 1H), 6.85 (dd, J=8.2, 1.9 Hz, 1H), 6.59 (m, 1H), 6.58 (d, J=1.8 Hz, 1H), 5.07 (s, 2H), 1.60 (s, 9H). ¹³C{¹H} NMR (CDC₃, 126 MHz) δ 165.4, 145.6, 143.6, 140.8, 133.2, 131.2, 130.1, 127.5, 127.4, 127.0, 126.5, 123.2, 122.6, 122.1, 115.8, 115.6, 81.1, 52.5, 28.3. HRMS (APCI) m/z: [M+H]+ calc'd for (C₂H₂₃ClNO₂S+) 424.1133; Found 424.1168.

### Example 3

2-Chloro-10-(3-methoxybenzyl)-10H-phenothiazine (4). General benzylation procedure provided a yellow oil (0.099 g, 28%). IR: 3021 cm⁻¹, 2967 cm⁻¹, 2851 cm⁻¹. ¹H NMR (CDCl₃, 500 MHz) δ 7.22-7.20 (m, 2H), 7.08 (dd, J=7.6, 1.6 Hz, 1H), 7.02-6.96 (m, 2H), 6.91-6.86 (m, 2H), 6.84 (dd, J=8.1, 2.0 Hz, 2H), 6.66 (dd, J=8.2, 1.2 Hz, 1H), 6.64 (d, J=2.1 Hz, 1H), 4.99 (s, 2H), 3.79 (s, 3H). ¹³C{¹H} NMR (CDCl₃, 126 MHz) δ 158.7, 145.8, 143.9, 133.1, 129.7, 127.7, 127.7, 127.4, 127.3, 126.9, 123.0, 122.9, 122.3, 121.9, 115.9, 115.7, 114.3, 113.8, 55.3, 52.1. HRMS (APCI) m/z: [M+H]+ calc'd for (C₂₀H₁₇ClNOS+) 353.0641; Found 353.0672.

### Example 4

Methyl 3-((2-chloro-10H-phenothiazin-10-yl)methyl)benzoate (5). General benzylation procedure provided a white solid (0.535 g, 20%). IR: 3100 cm⁻¹, 2949 cm⁻¹, 1713 cm⁻¹. ¹H NMR (CDCl₃, 500 MHz) δ 8.05 (t, J=1.8 Hz, 1H), 7.94 (dt, J=7.6, 1.5 Hz, 1H), 7.47 (m, 1H), 7.37 (t, J=7.7 Hz, 1H), 7.09 (dd, J=7.6, 1.6 Hz, 1H), 7.00-6.95 (m, 2H), 6.88 (td, J=7.5, 1.2 Hz, 1H), 6.84 (dd, J=8.2, 2.0 Hz, 1H), 6.61 (dd, J=8.1, 1.2 Hz, 1H), 6.59 (d, J=2.1 Hz, 1H), 5.06 (s, 2H), 3.91 (s, 3H). ¹³C{¹H} NMR (CDCl₃, 126 MHz) δ 166.8, 145.7, 143.6, 136.7, 133.1, 131.1, 130.9, 129.0, 128.6, 127.8, 127.5, 127.5, 127.1, 123.5, 123.2, 122.6, 122.3, 115.8, 115.6, 52.3. HRMS (APCI) m/z: [M+H]+ calc'd for (C₂₁H₁₇ClNO₂S+) 382.0669; Found: 382.0670.

### Example 5

4-((2-Chloro-10H-phenothiazin-10-yl)methyl)phenyl acetate (6). General benzylation procedure provided an off-white solid (0.127 g, 12%). IR: 3050 cm⁻¹, 2960 cm⁻¹, 1742 cm⁻¹. ¹H NMR (CDCl₃, 500 MHz) δ 7.32 (d, J=6.7 Hz, 2H), 7.11-7.08 (m, 3H), 7.02-6.98 (m, 2H), 6.90 (td, J=7.5, 1.2 Hz, 1H), 6.86 (dd, J=8.2, 2.0 Hz, 1H), 6.65 (dd, J=8.2, 1.1 Hz, 1H), 6.63 (d, J=2.0 Hz, 1H), 5.02 (s, 2H), 2.31 (s, 3H). ¹³C{¹H} NMR (CDCl₃, 126 MHz) δ169.5, 149.8, 145.7, 143.7, 133.5, 133.2, 127.6, 127.5, 127.5, 127.0, 123.2, 123.1, 122.5, 122.0, 122.0, 115.9, 115.6, 52.2, 21.2. HRMS (APCI) m/z: [M+H]+ calc'd for (C₂₁H₁₇ClNO₂S+) 382.0663; Found 382.0669.

### Example 6

2-Chloro-10-(3-nitrobenzyl)-10H-phenothiazine (7). Benzylation followed the general procedure with one addendum. After addition of 3-(bromomethyl)-nitrobenzene, potassium iodide (0.166 g, 1 mmol) was added and the reaction was stirred while covered in foil at room temperature overnight. The reaction is diluted with ether (ca. 2 solvent volumes) and poured into separatory funnel containing a 10% wt/wt solution of LiBr in DI water. The ether layer is carefully washed 2× with LiBr (aq) and then Brine (1× solvent volume), dried over sodium sulfate, filtered, and concentrated under reduced pressure. Crude material was then purified using an automated CombiFlash chromatograph (silica gel, 20-40 μm, gradient 30% ethyl acetate in hexane) producing a bright yellow product (1.16 g, 31%). IR: 3062 cm⁻¹, 2924 cm⁻¹, 1459 cm⁻¹. ¹H NMR (CDCl₃, 500 MHz) δ 8.24 (t, J=2.0 Hz, 1H), 8.13 (m, 1H), 7.68-7.64 (m, 1H), 7.50 (t, J=7.9 Hz, 1H), 7.14 (dd, J=7.6, 1.5 Hz, 1H), 7.04 (d, J=8.3 Hz, 1H), 7.03-6.99 (m, 1H), 6.93 (td, J=7.5, 1.2 Hz, 1H), 6.89 (dd, J=8.2, 2.0 Hz, 1H), 6.61 (d, J=1.2 Hz, 1H), 6.60 (t, J=1.7 Hz, 1H), 5.14 (s, 2H). ¹³C{¹H} NMR (CDCl₃, 126 MHz,) δ 148.7, 145.5, 143.4, 138.6, 133.2, 132.8, 129.9, 127.8, 127.5, 127.4, 124.0, 123.5, 122.9, 122.8, 122.6, 121.8, 115.7, 115.6, 51.8. HRMS (APCI) m/z: [M+H]+ calc'd for (C₁₉H₁₄ClN₂O₂S+) 369.0464; Found 369.0511.

### Example 7

4-((2-Chloro-10H-phenothiazin-10-yl)methyl)phenol (8). 4-((2-chloro-10H-phenothiazin-10-yl)methyl)phenyl acetate (6) (0.088 g, 0.23 mmol) was added to a stirred solution of KOH (excess) in a mixture of ethanol/water (1:1) and refluxed for 4 hours. After cooling, mixture was concentrated to half volume and extracted with ether. Organic layer was discarded, and the aqueous layer acidified to pH 2 and extracted into ether. Crude mixture was then concentrated under reduced pressure and purified using an automated CombiFlash chromatograph (silica gel, 20-40 μm, gradient 30% ethyl acetate in hexane) providing a white solid that rapidly turns purple (131 mg, 21%). IR: 3321 cm⁻¹, 3062 cm⁻¹, 2962 cm⁻¹. ¹H NMR (CDCl₃, 500 MHz) δ 7.19 (t, J=7.7 Hz, 1H), 7.06 (dd, J=7.6, 1.6 Hz, 1H), 7.03-6.94 (m, 3H), 6.88 (td, J=7.5, 1.2 Hz, 1H), 6.86-6.82 (m, 1H), 6.81-6.76 (m, 2H), 6.65 (dd, J=8.2, 1.2 Hz, 1H), 6.62 (d, J=2.1 Hz, 1H), 4.98 (s, 2H), 2.02 (s, 1H). ¹³C{¹H} NMR (CDCl₃, 126 MHz) δ 154.6, 145.7, 143.9, 133.1, 129.7, 127.9, 127.9, 127.4, 127.4, 126.9, 123.0, 123.0, 122.3, 121.9, 115.9, 115.8, 115.7, 115.2, 52.1. HRMS (APCI) m/z: [M+H]+ calc'd for (C₁₉H₁₅ClNOS⁺) 340.0536; Found 340.0474.

### Example 8

3-((2-Chloro-10H-phenothiazin-10-yl)methyl)aniline (9). 2-chloro-10-(3-nitrobenzyl)-10H-phenothiazine (7) (1.16 g, 3.1 mmol) was dissolved in EtOAc. Tin(II) chloride dihydrate (6.9 g, 31 mmol) was added and the mixture refluxed for 2 h. The mixture was cooled to room temperature and poured into ice, and the pH was adjusted to 10. The resulting slurry was extracted with EtOAc (3×50 mL) and the combined organic fractions washed with brine (1×50 mL), dried over sodium sulfate, filtered, and concentrated under reduced pressure resulting in a white solid (1.17 g, quant.). IR: 3365 cm⁻¹, 3015 cm⁻¹, 2968 cm⁻¹, 1717 cm⁻¹. ¹H NMR (CDCl₃, 500 MHz) δ 7.17-7.13 (m, 1H), 7.06 (dd, J=7.5, 1.6 Hz, 1H), 7.01-6.98 (m, 1H), 6.98-6.95 (m, 1H), 6.88 (td, J=7.5, 1.2 Hz, 1H), 6.84 (dd, J=8.2, 2.0 Hz, 1H), 6.74-6.70 (m, 1H), 6.67-6.62 (m, 4H), 4.96 (s, 2H), 4.32 (br, 2H). ¹³C{¹H} NMR (CDCl₃, 126 MHz) δ 145.7, 145.7, 143.8, 137.4, 133.1, 129.9, 127.5, 127.2, 126.8, 122.9, 122.7, 122.3, 121.5, 117.4, 115.8, 115.6, 114.6, 113.4, 52.8. HRMS (APCI) m/z: [M+H]+ calc'd for (C₁₉H₁₆ClN₂S⁺) 339.0723; Found 339.0822.

### Example 9

5-(2-chloro-10H-phenothiazin-10-yl)pentanenitrile (10). To a suspension of sodium hydride (0.200 g, 5 mmol) in anhydrous THF was added a solution of 2-chloro-10H-phenothiazine (1.165 g, 5 mmol) dropwise at room temperature in a round bottom flask. Solution was stirred for 0.5 hours before addition of valeronitrile (0.58 mL, 5 mmol) in a single portion. The mixture stirred for 48 hours before being concentrated, extracted into ether, washed with brine (3×50 mL), and extracted with chloroform (2×50 mL). The crude mixture was concentrated under reduced pressure and purified using slow gradient of hexane:ethyl acetate using an automated CombiFlash chromatograph (silica gel, 20-40 μm) resulting in a yellow solid (1.3 g, 88%). IR: 3075 cm⁻¹, 2940 cm⁻¹, 2255 cm⁻¹. ¹H NMR (CDCl₃, 500 MHz) δ 7.21-7.18 (m, 1H), 7.17 (dd, J=7.5, 1.5 Hz, 1H), 7.07 (d, J=8.2 Hz, 1H), 6.97 (td, J=7.5, 1.1 Hz, 1H), 6.92 (dd, J=8.2, 2.0 Hz, 1H), 6.87 (dd, J=8.2, 1.1 Hz, 1H), 6.83 (d, J=2.0 Hz, 1H), 3.92 (t, J=6.4 Hz, 2H), 2.35 (t, J=7.1 Hz, 2H), 1.99-1.92 (m, 2H), 1.83-1.75 (m, 2H). ¹³C{¹H}NMR (CDCl₃, 126 MHz) δ 146.4, 144.3, 133.3, 128.2, 127.8, 127.5, 125.6, 124.3, 123.3, 122.7, 119.3, 116.0, 115.9, 46.2, 25.6, 22.7, 16.9. HRMS (APCI) m/z: [M+H]+ calc'd for (C₁₇H₁₆ClN₂S) 315.0717; Found 315.0720.

### Example 10

5-(2-Chloro-10H-phenothiazin-10-yl)pentanamide (11). 5-(2-chloro-10H-phenothiazin-10-yl)pentanenitrile (10) was dissolved in 7 mL concentrated sulfuric acid and stirred for 2.5 hours in a round bottom flask. The solution was then poured into 20 mL ice-water mixture and basified to pH 10 with concentrated ammonium hydroxide (caution: add slow and keep solution cold). Mixture was then extracted with EtOAc (3×100 mL), washed with brine (1×100 mL), dried over sodium sulfate, and concentrated in vacuo to give the title compound (95%). IR: 3375 cm⁻¹, 3065 cm⁻¹, 2960 cm⁻¹, 1650 cm⁻¹, 1590 cm⁻¹. ¹H NMR (CDCl₃, 500 MHz) δ 7.16 (td, J=7.7, 1.6 Hz, 1H), 7.13 (dd, J=7.7, 1.5 Hz, 1H), 7.03 (d, J=8.2 Hz, 1H), 6.94 (t, J=7.5 Hz, 1H), 6.89 (dd, J=8.1, 2.0 Hz, 1H), 6.85 (dd, J=8.2, 1.1 Hz, 1H), 6.82 (d, J=2.0 Hz, 1H), 5.63 (br, 1H), 5.47 (br, 1H), 3.89-3.80 (m, 2H), 2.22 (t, J=7.3 Hz, 2H), 1.89-1.79 (m, 2H), 1.79-1.71 (m, 2H).

¹³C{¹H} NMR (CDCl₃, 126 MHz) δ 175.1, 146.5, 144.5, 133.3, 128.0, 127.6, 127.5, 124.9, 123.7, 123.0, 122.4, 115.9, 115.9, 47.0, 35.2, 26.0, 22.8. HRMS (APCI) m/z: [M+H]+ Calc'd (C₁₇H₁₈N₂OS+) 333.0823; Found 333.0898.

### Example 11

N-phenylacrylamide (12). General acylation procedure provided a white solid (0.380 g, 86%). IR: 3265 cm⁻¹, 3075 cm⁻¹, 1674 cm⁻¹. ¹H NMR (DMSO-d₆, 500 MHz) δ 10.12 (br, 1H), 7.68-7.61 (m, 2H), 7.31-7.28 (m, 2H), 7.05 (tt, J=7.4, 1.2 Hz, 1H), 6.45-640 (m, 1H), 6.24 (dd, J=17.0, 2.0 Hz, 1H), 5.73 (dd, J=10.1, 2.0 Hz, 1H). ¹³C{¹H} NMR (DMSO-d₆, 126 MHz) δ 163.6, 139.5, 132.3, 129.2, 127.3, 123.9, 119.8. HRMS (APCI) m/r. [(M+H)+] calcd for (C₉H₁₀NO⁺) 148.0781; Found 148.0976.

### Example 12

N-(4-fluorophenyl)acrylamide (13). General acylation procedure provided an off-white solid (0.395 g, 71%). IR: 3468 cm⁻¹, 3105 cm⁻¹, 1634 cm⁻¹. ¹H NMR (CDCl₃, 500 MHz) δ 7.54 (dd, J=8.9, 4.8 Hz, 2H), 7.33 (br, 1H), 7.07-6.99 (m, 2H), 6.44 (dd, J=16.9, 1.2 Hz, 1H), 6.24 (dd, J=16.8, 10.2 Hz, 1H), 5.78 (dd, J=10.3, 1.2 Hz, 1H). ¹³C{¹H} NMR (CDC₃, 126 MHz) δ 163.4, 130.9, 128.1, 121.8, 121.7, 115.8, 115.6. HRMS (APCI) m/z: [(M+H)+] calcd for (C₉H₉NO⁺) 166.0630; Found 166.0780.

### Example 13

Bis(4-fluorophenyl)amine (S1). The 4-fluoroaniline (0.333 g, 3 mmol) and corresponding 4-fluorophenyl boronic acid (0.834 g, 6 mmol) were added into DCM with TEA (0.606 mL, 6 mmol) and Cu(OAc)₂ (0.597 g, 3 mmol) with 4 Å molecular sieves. The solution was stirred at room temperature under air for 16 hours. The resulting mixture was filtered through a silica plug and the solvent was evaporated under reduced pressure. The crude mixture was purified via automated CombiFlash chromatography (silica gel, 20-40 microns, gradient 15% ethyl acetate and 85% hexane) providing a yellow oil (0.270 g, 44%). IR: 3400 cm⁻¹, 3097 cm⁻¹. ¹H NMR (DMSO-d₆, 500 MHz) δ 8.04 (s, 1H), 7.07-7.02 (m, 4H), 7.02-6.98 (m, 4H). ¹³C{¹H} NMR (DMSO-d₆, 126 MHz) δ 157.6, 155.7, 140.7, 140.7, 118.6, 118.5, 116.2, 116.1. HRMS (APCI) m/z. [(M+H)+] calcd for (C₁₂H₁₀F₂N+) 206.0737; Found 206.0862.

### Example 14

N,N-bis(4-fluorophenyl)acrylamide (14). General acylation procedure provided an off-white solid (0.337 g, 90%). IR: 3075 cm⁻¹, 1669 cm⁻¹. ¹H NMR (CDCl₃, 500 MHz) δ 7.24-7.15 (m, 4H), 7.08 (d, J=8.8 Hz, 4H), 6.48 (dd, J=16.8, 1.8 Hz, 1H), 6.17 (dd, J=16.8, 10.3 Hz, 1H), 5.67 (dd, J=10.3, 1.9 Hz, 1H). ¹³C{1H} NMR (DMSO-d₆, 151 MHz, 338K) δ 165.2, 161.2 (d, J=244.7 Hz), 139.2, 130.2, 129.7, 128.6, 116.6, 116.5. HRMS (APCI) m/z [(M+H)+] calcd for (C₁₅H₁₂F₂NO+) 260.0842; Found 260.0888.

### Example 15

3-(2-chloro-10H-phenothiazin-10-yl)propenamide (15). General Michael-addition procedure provided a white solid (0.065 g, 25%). IR: 3392 cm⁻¹, 3025 cm⁻¹, 2990 cm⁻¹, 1645 cm⁻¹, 1607 cm⁻¹. ¹H NMR (DMSO-d₆, 500 MHz) δ 8.13 (d, J=8.1, 2H), 7.72 (d, J=1.8 Hz, 1H), 7.61 (d, J=8.2 Hz, 1H), 7.47-7.44 (m, 1H), 7.35 (s, 1H), 7.23-7.16 (m, 2H), 6.87 (s, 1H), 4.58 (t, J=6.7 Hz, 2H), 2.55 (t, J=6.7 Hz, 2H). ¹³C{¹H} NMR (DMSO-ds, 126 MHz) δ 172.5, 140.9, 140.6, 130.7, 126.6, 122.1, 122.0, 121.5, 120.8, 119.8, 119.4, 110.2110.0, 334.8. HRMS (APCI) m/z: [(M+H)+] calcd for (C₁₅H₁₄ClN₂OS+) 305.0515; Found 305.0777.

### Example 16

3-(2-chloro-10H-phenothiazin-10-yl)-N,N-dimethylpropanamide (16). General Michael-addition procedure provided an off-white solid (0.332 g, 85%). IR: 3070 cm⁻¹, 2885 cm⁻¹, 1595 cm⁻¹. ¹H NMR (CDCl₃, 500 MHz) δ 8.05 (d, J=7.8, 1H), 7.99 (d, J=8.2 Hz, 1H), 7.49-7.43 (m, 3H), 7.25 (dd, J=6.4, 1.6 Hz, 1H), 7.21 (dd, J=8.2, 1.8 Hz, 1H), 4.69-4.64 (m, 2H), 2.91 (s, 3H), 2.84-2.78 (m, 2H), 2.71 (s, 3H). ¹³C{¹H} NMR (CDCl₃, 126 MHz) δ 170.5, 140.6, 140.3, 131.6, 126.1, 122.5, 121.6, 121.2, 120.3, 119.6, 119.6, 108.9, 108.8, 39.4, 37.0, 35.4, 31.9. HRMS (APCI) m/z: [(M+H)+] calcd for (C₁₇H₁₈ClN₂OS+) 333.8450; Found 333.9107.

### Example 17

3-(2-chloro-10H-phenothiazin-10-yl)-N-phenylpropenamide (17). General Michael-addition procedure provided a white oil (0.097 g, 41%). IR: 3761 cm⁻¹, 3110 cm⁻¹, 2880 cm⁻¹, 1672 cm⁻¹. ¹H NMR (CDCl₃, 500 MHz) δ 8.04 (d, J=7.8, 1H), 7.97 (d, J=8.2 Hz, 1H), 7.43 (m, 1H), 7.34 (dt, J=8.4, 0.9 Hz, 1H), 7.26-7.23 (m, 1H), 7.20 (dd, J=8.2, 1.8 Hz, 1H), 7.18-7.13 (m, 1H), 7.10 (m, 1H), 6.86-6.82 (m, 2H), 6.64-6.60 (m, 1H), 6.27-6.22 (m, 2H), 4.64 (t, J=6.6 Hz, 2H), 2.59 (t, J=6.6 Hz, 2H). ¹³C{¹H} NMR (CDC₃, 126 MHz) δ 170.5, 140.4, 140.2, 136.4, 131.6, 129.8, 129.7, 128.6, 128.3, 127.4, 126.2, 122.3, 121.4, 121.1, 120.2, 119.7, 116.3, 116.1, 109.1, 39.9, 32.7. HRMS (APCI) m/z: [(M+H)+] calcd for (C₂₁H₁₈ClN₂OS+) 381.0828; Found 381.0941.

### Example 18

3-(2-chloro-10H-phenothiazin-10-yl)-N-(4-fluorophenyl)-N-methylpropanamide (18). General Michael-addition procedure provided a white solid (0.170 g, 38%). ¹H NMR (CDCl₃, 500 MHz) δ 7.12 (dd, J=7.6, 1.5 Hz, 1H), 7.08 (t, J=7.8 Hz, 1H), 7.01 (d, J=8.2 Hz, 1H), 7.00-6.90 (m, 5H), 6.88 (d, J=8.1 Hz, 1H), 6.75 (d, J=8.1 Hz, 1H), 6.71 (d, J=2.0 Hz, 1H), 4.15 (t, J=25.5 Hz, 2H), 3.25 (s, 3H), 2.53 (t, J=6.9 Hz, 2H). ¹³C{¹H} NMR (CDCl³, 126 MHz) δ 170.69, 161.72 (d, J=248.4 Hz), 146.20, 139.43 (d, J=3.3 Hz), 133.36, 128.83, 128.77, 127.92, 127.53, 124.66, 123.38, 123.10, 122.47, 116.77, 116.59, 115.56, 115.45, 43.64, 37.46, 31.28.

### Example 19

3-(2-chloro-10H-phenothiazin-10-yl)-N,N-bis(4-fluorophenyl)propanamide (19). General Michael-addition procedure provided a light pink solid (0.350 g, 80%). IR: 3015 cm⁻¹, 2960 cm⁻¹, 1664 cm⁻¹. ¹H NMR (CDCl₃, 500 MHz) δ 7.24-7.16 (m, 3H), 7.13-7.06 (m, 2H), 7.02-6.88 (m, 8H), 6.79 (dd, J=8.2, 1.2 Hz, 1H), 6.75 (d, J=2.0 Hz, 1H), 4.27 (t, J=6.4 Hz, 2H), 2.71 (t, J=6.4 Hz, 2H). ¹³C{¹H} NMR (CDCl₆, 126 MHz) δ 170.8, 146.3, 144.2, 138.2, 138.2, 133.5, 129.8, 128.2, 128.0, 127.7, 127.6, 125.0 123.7, 123.2, 122.6, 116.7, 115.9, 115.8, 115.7, 115.6, 43.7, 32.0. HRMS (APCI) m/z: [(M+H)+] calcd for (C₂₇H₂₀F₂N₂OS⁺) 494.0845; Found 494.0735.

Biological Studies:

Materials and Reagents. Human 20S proteasome and fluorogenic substrates N-succinyl-Leu-Leu-Val-Tyr-7-amido-4-methylcoumarin (Suc-LLVY-AMC), carboxyl benzyl-Leu-Leu-Glu-7-amido-4-methylcoumarin (Z-LLE-AMC), tert-butyloxycarbonyl-Leu-Arg-Arg-7-amido-4-methylcoumarin (Boc-LRR-AMC), and bortezomib were obtained from Boston Biochem, Inc. (Cambridge, MA). The PVDF membrane, Clarity western ECL reagent, blocking grade milk, and precast sodium dodecyl sulfate gels were from Bio-Rad (Hercules, CA). The recombinant wild type α-synuclein was obtained from Abcam (Cambridge, MA). Rabbit polyclonal anti-α-synuclein, mouse monoclonal anti-α-synuclein and goat anti-rabbit HRP-linked antibody were purchased from Santa Cruz Biotechnologies (Dallas, TX). The anti-mouse HRP-linked antibody was purchased from Cell Signaling Technology (Danvers, MA). Unless otherwise noted, chemicals were purchased from commercial suppliers and used without further purification.

Molecular docking studies. Docking was performed using Autodock vina, supported through computational resources and services provided by the Institute for Cyber-Enabled Research at Michigan State University. The crystal structure of the closed gate human proteasome (h20S) was obtained from the PDB database (PDB ID: 4R3O). Molecules were generated in Perkin Elmer's Chem3D, minimized using the MM2 force field, and converted to PDB. These molecules were uploaded to PyRx and converted to ligand pdbqt files. Small molecule ligands were then docked against the entirety of the h20S proteasome (grid box 153.2×138.0×189.4 Å) three times with exhaustiveness set to 1000. Active compounds displayed a preference for the α-rings. Individual poses were manually inspected using Pymol and BIOVIA Discovery Studio 2020.

Drug Stock Preparation: All compounds were dissolved in DMSO to make the drug stocks. The percent DMSO used in each assay are as follows: 1% DMSO in the small peptide assay, 2% in the purified α-synuclein digestion, and 0.05% in the in-cell A-53T α-synuclein degradation.

20S Proteasome Small Peptide Assay: Activity assays were carried out in a black flat/clear bottom 96 well plate with a 200 mL volume. Concentrations of the potential activators, varying from 80 μM to 1.25 μM, were added to the well that contained 1 nM 20S proteasome purchased from either Boston Biochem (catalog number E-360) or Life Sensors (catalog number ps020), in a 50 nM Tris-HCl and 100 nM NaCl buffer at a pH of 7.8. This solution was left to incubate at 37° C. for 15 minutes. For the assay for the overall proteasome activity, 13.33 μM of each small peptide substrate is added. The 96 well plate is then placed into a plate reader (SpectraMax M5e spectrometer) and the plate reader will take a fluorescent reading every 5 minutes for an hour at 37° C. The enzymatic activity of the 20S proteasome is quantified using the fluorescent output at 380-460 nm, which is converted to max fold increase over the untreated sample. The activity of the individual catalytic assays is identical to the assay that assesses the overall activity of the 20S proteasome except only one small peptide substrate is added. To evaluate the activity of the CT-L and the Casp-L sites, 10 μM of the Suc-LLVY-AMC (Boston Biochem S-280) and Z-LLE-AMC (Boston Biochem S-230) were added, respectively. To evaluate the activity of the T-L site, 20 μM of the Boc-LRR-AMC (Boston Biochem S-300) is added.

Digestion of purified α-synuclein: Degradation assays were carried out in a 50 μL reaction volume of 50 nM Tris-HCl and 100 nM NaCl buffer at a pH of 7.8, 0.5 μM of purified α-synuclein, and 7.5 nM purified human 20S proteasome. The 20S proteasome was first diluted to 8.56 nM in the Tris-HCl buffer and 1 μL of the test compounds or DMSO was added. This mixture was incubated for 20 minutes at 37° C. After this incubation, 5 μL of a 5 μM stock of α-synuclein was added. The degradation mixture was incubated at 37° C. for 5 hours. The reaction was stopped with addition of concentrated SDS loading buffer and boiled for 10 minutes. After the samples were boiled, they were resolved on a 4-20% Tris-glycine SDS-PAGE gel and visualized using silver staining.

Tet-One A53T plasmid: Using CloneAMP HiFi (Clonetech 639298) PCR reagent system, 12.5 mL of the CloneAmp HiFi PCR Premix was added to 5-7.5 pmol of primer A (the amount will be dependent on the dilution of the primers) (ttttataggcgccctggttt), primer B (tgtatttagaaaaataaacaaata), and 100 ng of A53T template were added and sterilized deionized water was added to bring the mixture volume up to 25 mL. The mixture was then put into a thermal cycler and the following cycle was carried out: 96° C. for 3 minutes, then 30 cycles of 96° C. for 10 seconds, 64° C. for 10 seconds, and 72° C. for 66 seconds, and one cycle of 72° C. for 10 minutes.

Using the In-Fusion cloning system (Clonetech, catalog number 638920), 5 mL of 5× In-Fusion HD Enzyme premix, 50 to 200 ng of Tet-One vector that was linearized with NotI and PstI, 10 to 200 ng of A53T PCR fragment was combined, and deionized water was added until the reaction mixture volume was 10 mL. The mixture was then incubated for 15 minutes at 50° C., and the resulting ligated plasmids were grown in DH5α E. coli cells. The resulting plasmids were validated through sanger sequencing.

In cell degradation of A53T α-synuclein: In a 6 well plate, Hek293T cells were grown to 80% confluency and then transfected using Xfect biopolymer transfection system with the Tet-One A53T α-synuclein plasmid. 24 hours post transfection, 100 ng/mL of doxycycline was added to induce A53T α-synuclein expression. After 24 hours of protein induction, media was removed that contained the doxycycline and was washed with PBS. Media with the desired drug concentration or DMSO was added and treated for 10 hours. After the cells were treated with the test compounds or DMSO, the cells were scraped and the pelleted (300 g for 5 minutes). The supernatant was taken off and the cell pellet was washed with ice cold PBS and then lysed with RIPA buffer with added protease inhibitors. The cell lysate was incubated for 20 minutes on ice. The lysate was centrifuged for 15 minutes at 500 g and then the supernatant was collected. Concentrated SDS loading buffer was added and the samples were boiled for 10 minutes. The samples were then resolved on a 4-20% Tris-glycine SDS-PAGE gel and immunoblotted with mouse monoclonal anti α-synuclein IgG (1:2000) and anti-mouse HRP-linked IgG (1:2000). Blots were developed with ECL Westem reagent and imaged with an Azure Biosystems 3000 imager.

The disclosure provides for the following example embodiments, the numbering of which is not to be construed as designating levels of importance:

Embodiment 1 relates to a compound of the formula (I):

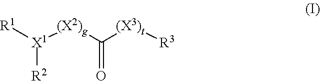

or a pharmaceutically acceptable salt, polymorph, prodrug, solvate or clathrate thereof, wherein:
R¹ is hydrogen, alkyl, cycloalkyl, aryl, heterocyclyl or heteroaryl;
R² is hydrogen, alkyl, cycloalkyl, aryl, heterocyclyl or heteroaryl;
R³ is aryl or heteroaryl;
X¹ is N or CR⁵, wherein R⁵ is absent, hydrogen, alkyl, heterocyclyl or aryl;
X² is alkyl or alkenyl;
X³ is alkyl or alkenyl;
g is 0 or 1; and
t is 0 or 1;
provided that g and t are not simultaneously 0. In some examples, g is 0 and t is 1. In other examples, g is 1 and t is 0.

Embodiment relates to the compound of Embodiment 1, wherein the compound of the formula (I) is a compound of the formula (Ia):

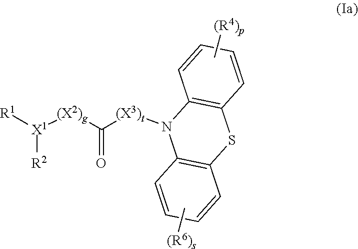

or a pharmaceutically acceptable salt, polymorph, prodrug, solvate or clathrate thereof, wherein:
R¹ is hydrogen, alkyl, cycloalkyl, aryl, heterocyclyl or heteroaryl;
R² is hydrogen, alkyl, cycloalkyl, aryl, heterocyclyl or heteroaryl;
R³ is aryl or heteroaryl;
X¹ is N or CR⁵, wherein R⁵ is absent, hydrogen, alkyl, heterocyclyl or aryl;
X² is alkyl or alkenyl;
X³ is alkyl or alkenyl;
g is 0 or 1; and
t is 0 or 1;
provided that g and t are not simultaneously 0;
R⁴ is alkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R⁶ is alkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
p is 0, 1, 2, 3 or 4; and
s is 0, 1, 2, 3 or 4.

Embodiment 3 relates to the compound of Embodiment 1, wherein the compound of formula (I) is a compound of the formula (Ib) or (Ic):

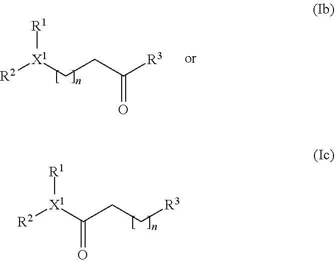

or a pharmaceutically acceptable salt, polymorph, prodrug, solvate or clathrate thereof,
wherein:
n is 0, 1 or 2.

Embodiment 4 relates to the compound of Embodiment 1, wherein the compound of formula (I) is a compound of the formula (Id) or (Ie):

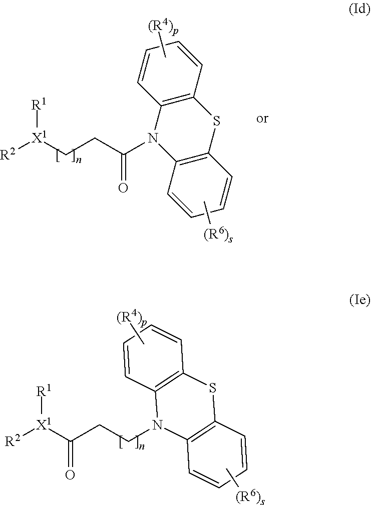

or a pharmaceutically acceptable salt, polymorph, prodrug, solvate or clathrate thereof,
wherein:
n is 0, 1 or 2.

Embodiment 5 relates to the compound of Embodiment 1, wherein the compound of formula (I) is a compound of the formula (If):

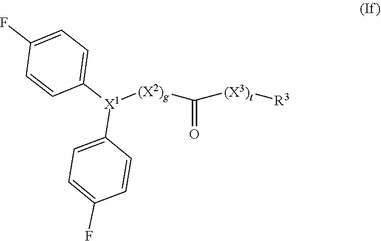

or a pharmaceutically acceptable salt, polymorph, prodrug, solvate or clathrate thereof, wherein:
R³ is aryl or heteroaryl;
X¹ is N or CR⁵, wherein R⁵ is absent, hydrogen, alkyl, heterocyclyl or aryl;
X² is alkyl or alkenyl;
X³ is alkyl or alkenyl;
g is 0 or 1; and
t is 0 or 1;
provided that g and t are not simultaneously 0.

Embodiment 6 relates to the compound of Embodiment 5, wherein g is 0 and t is 1.

Embodiment 7 relates to the compound of Embodiment 5, wherein g is 1 and t is 0

Embodiment 8 relates to the compound of Embodiment 1, wherein the compound of the formula (I) is a compound of the formula (Ig):

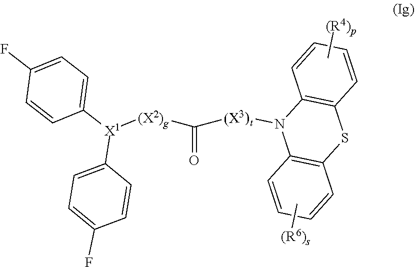

or a pharmaceutically acceptable salt, polymorph, prodrug, solvate or clathrate thereof, wherein:
X¹ is N or CR⁵, wherein R⁵ is absent, hydrogen, alkyl, heterocyclyl or aryl;
X² is alkyl or alkenyl;
X³ is alkyl or alkenyl;
g is 0 or 1; and
t is 0 or 1;
provided that g and t are not simultaneously 0

Embodiment 9 relates to the compound of Embodiment 8, wherein g is 0 and t is 1.

Embodiment 10 relates to the compound of Embodiment 8, wherein g is 1 and t is 0

Embodiment 11 relates to the compound of Embodiment 1, wherein the compound of formula (I) is a compound of the formula (Ih) or (Ii):

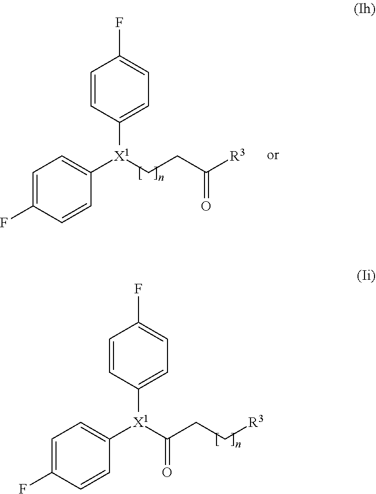

or a pharmaceutically acceptable salt, polymorph, prodrug, solvate or clathrate thereof,
wherein:
n is 0, 1 or 2.

Embodiment 12 relates to the compound of Embodiment 1, wherein the compound of the formula (I) is a compound of the formula (Ij) or (Ik):

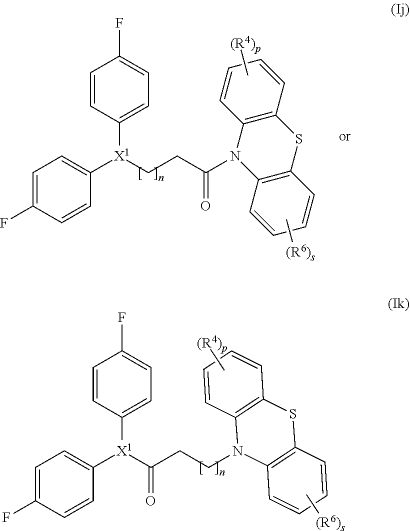

or a pharmaceutically acceptable salt, polymorph, prodrug, solvate or clathrate thereof,
wherein:
n is 0, 1 or 2.

Embodiment 13 relates to the compound of Embodiment 1, wherein the compound of the formula (I) is a compound of the formula (II):

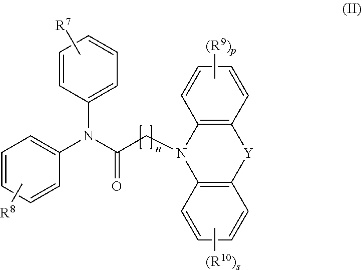

or a pharmaceutically acceptable salt, polymorph, prodrug, solvate or clathrate thereof,
wherein:
n is 0, 1 or 2;
Y is S(O)ₓ, wherein x is 0, 1 or 2, O, CH₂, or Y is N—R¹¹, wherein R¹¹ is a carbon with at least one halo, alkyl, aryl, acyl or heterocyclyl;
R⁷, R⁸, R⁹, and R¹⁰ are each independently halo, a carbon with at least one halo, alkyl, aryl, acyl or heterocyclyl;
p is 0, 1, 2, 3 or 4; and
s is 0, 1, 2, 3 or 4.

Embodiment 14 relates to the compound of Embodiment 1, wherein the compound of the formula (I) is a compound of the formula:

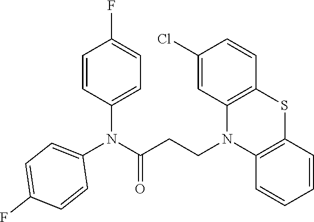 [SMILES: O=C(CCN1c2ccccc2Sc2ccc(Cl)cc21)N(c1ccc(F)cc1)c1ccc(F)cc1]

pharmaceutically acceptable salts, polymorphs, prodrugs, solvates or clathrates thereof.

Embodiment 15 relates to a compound of the formula (III):

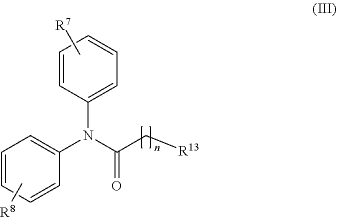

or a pharmaceutically acceptable salt, polymorph, prodrug, solvate or clathrate thereof,
wherein:
n is 0, 1 or 2;
R⁷ and R⁸ are each independently halo, a carbon with at least one halo, alkyl, aryl, acyl or heterocyclyl; and
R¹³ is a heterocyclyl group of the formula:

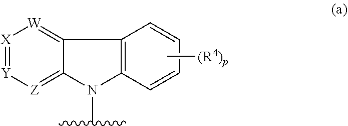

wherein W is N or C—R¹⁴; X is N or C—R¹⁴; Y is N or C—R¹⁴; and Z is N or C—R¹⁴; wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R⁴ is alkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4;

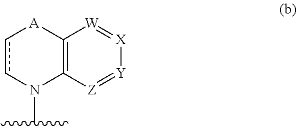

wherein the dashed line can represent a double bond; A is S(O)_{Y}, wherein x is 0, 1 or 2; O; C(R¹⁴)₂, wherein each R¹⁴ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or A is N—R¹¹, wherein R¹¹ is a carbon with at least one halo, alkyl, aryl, acyl or heterocyclyl;

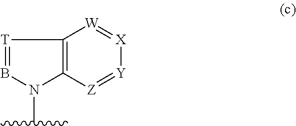

wherein T is CR¹⁴, wherein each R¹⁴ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or T is N; and
B is C—R¹⁴, wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocycyl; or B is N;

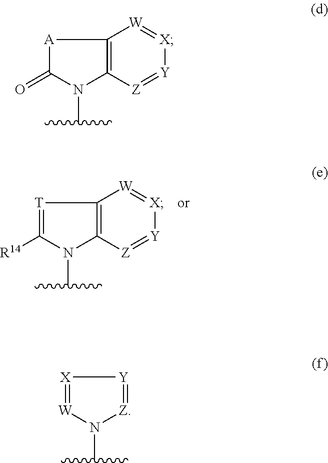

Embodiment 16 relates to the compound of Embodiment 15, wherein the compounds of the formula (III) are compounds of the formulae:

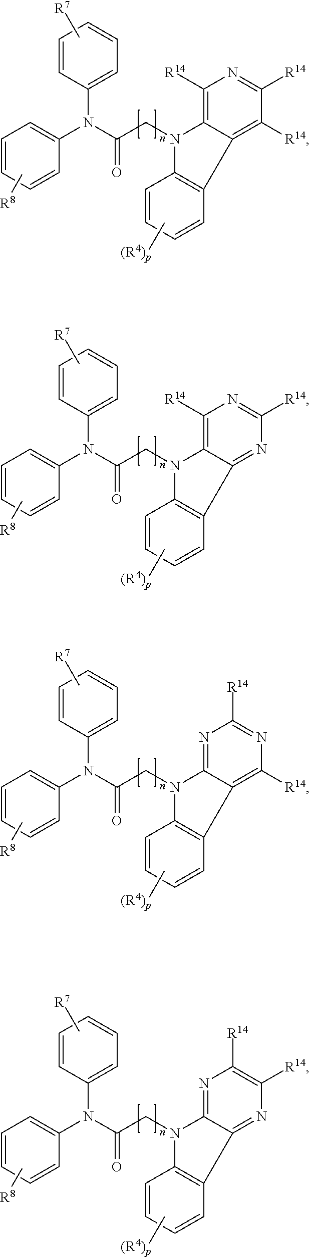

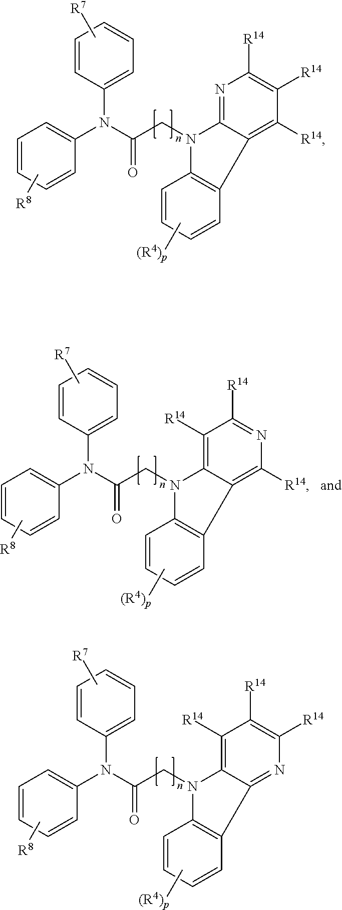

or a pharmaceutically acceptable salt, polymorph, prodrug, solvate or clathrate thereof.

Embodiment 17 relates to the compound of Embodiment 15, wherein the compounds of the formula (III) are compounds of the formulae:

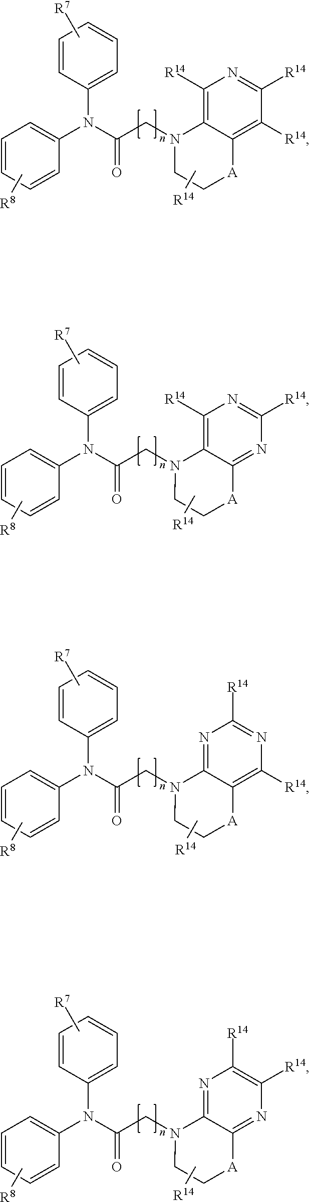

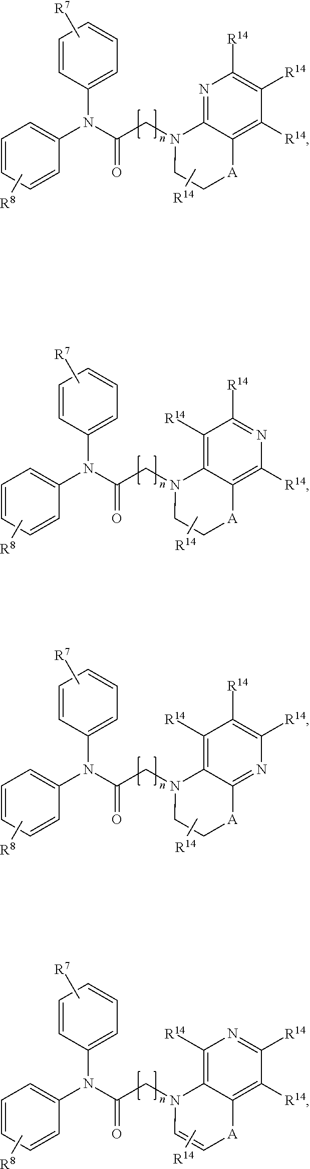

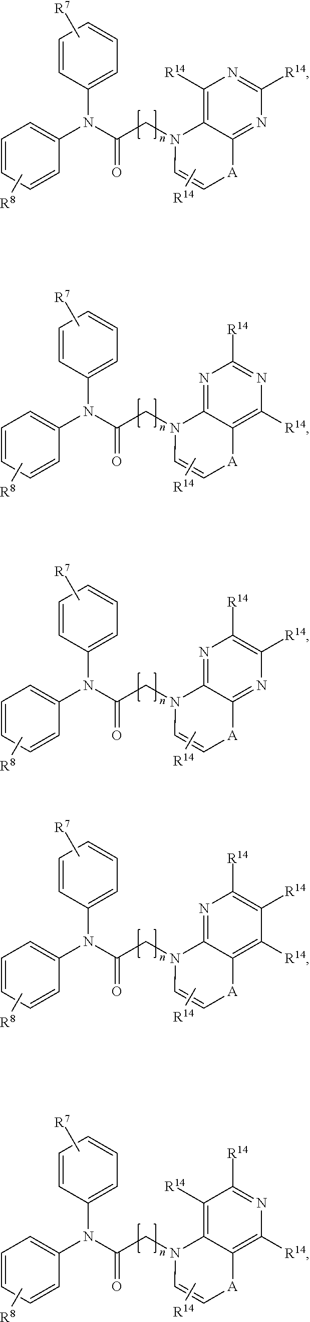

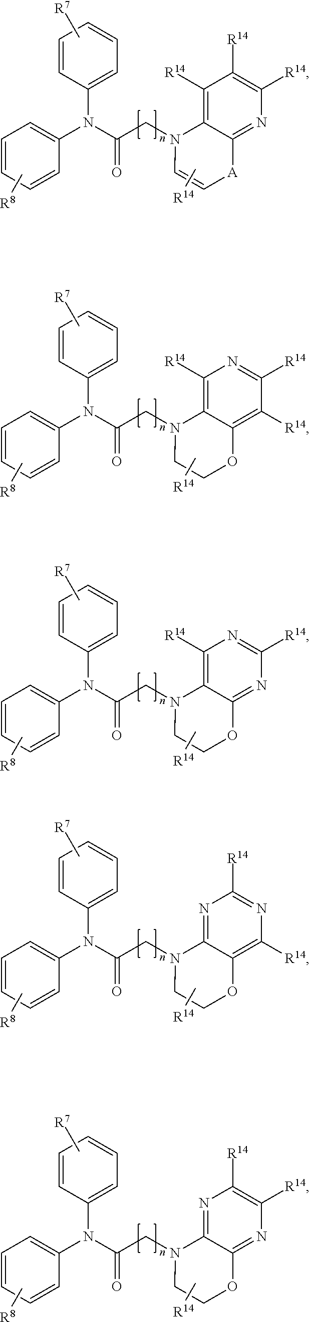

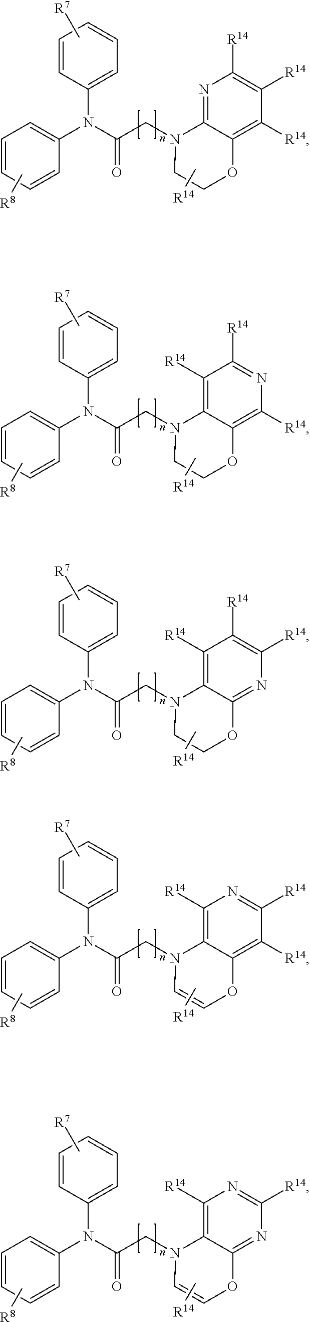

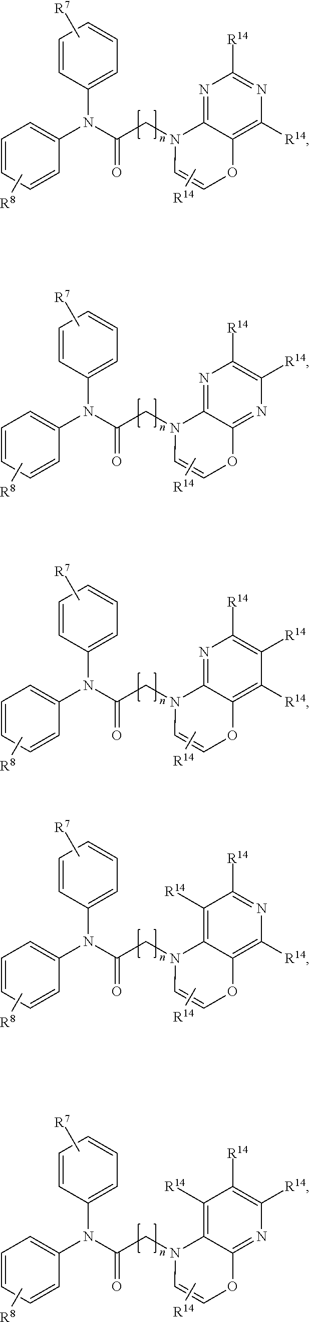

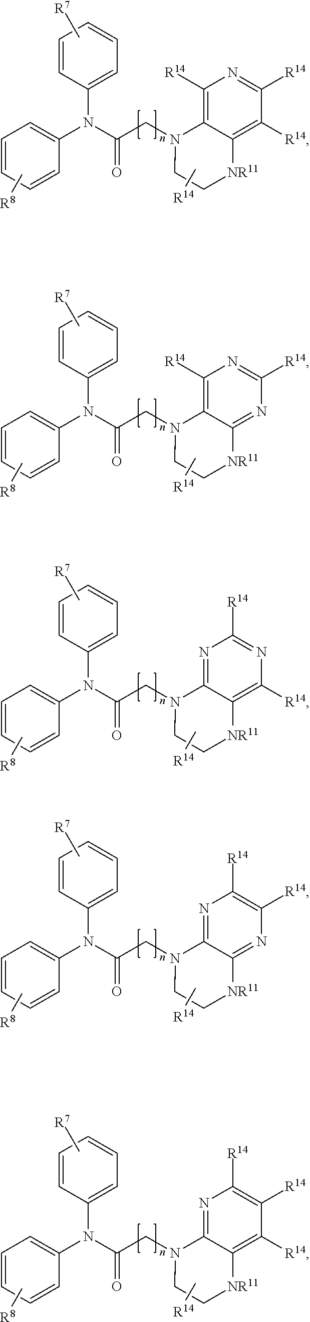

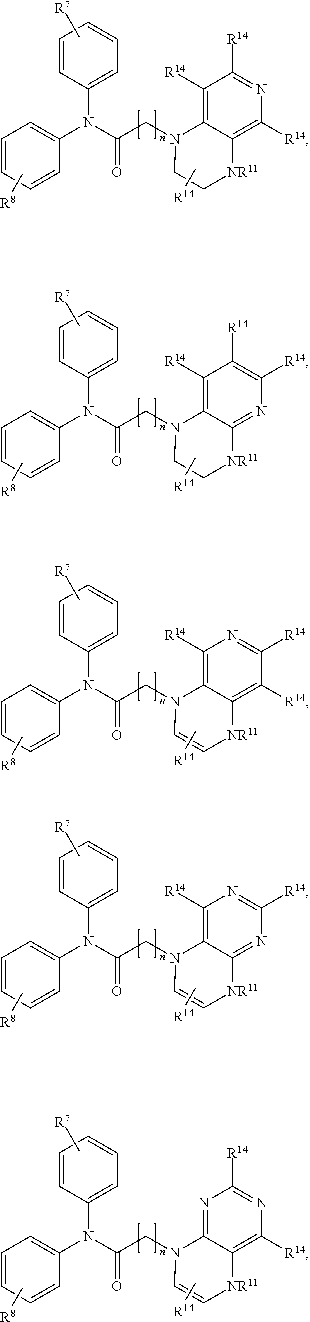

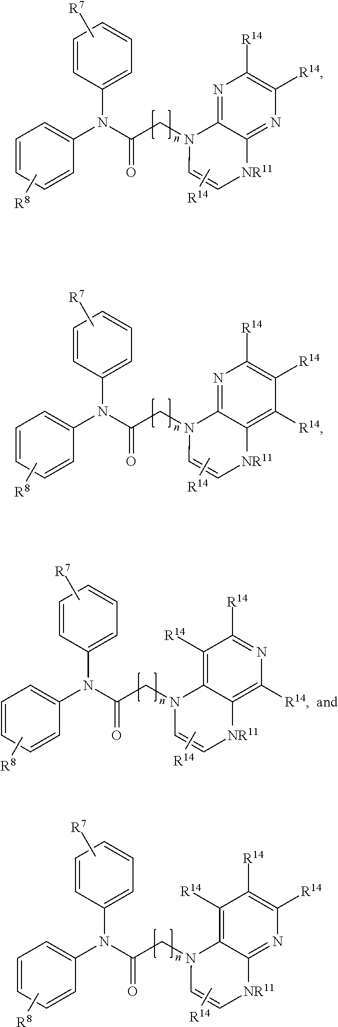

or a pharmaceutically acceptable salt, polymorph, prodrug, solvate or clathrate thereof.

Embodiment 18 relates to the compound of Embodiment 15, wherein the compounds of the formula (III) are compounds of the formulae:

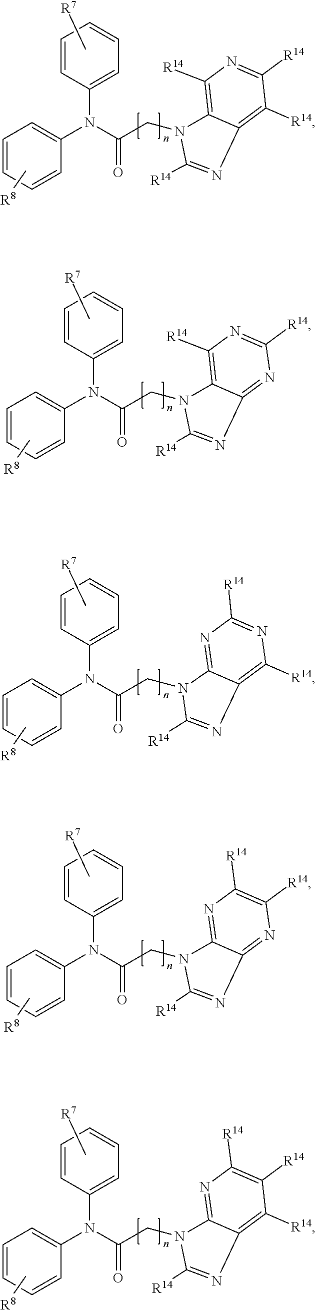

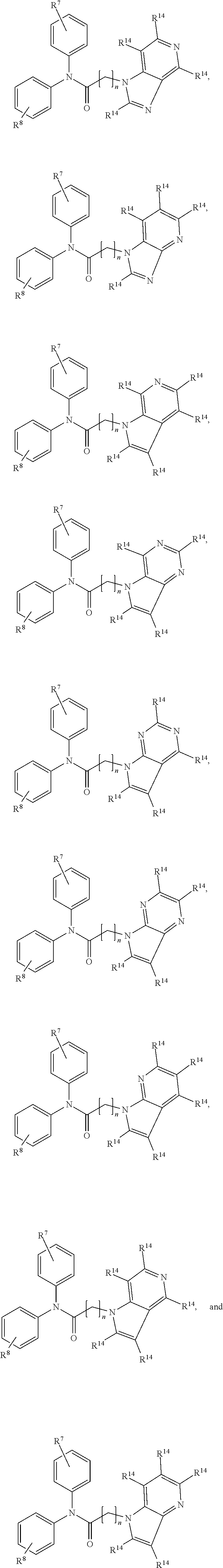

or a pharmaceutically acceptable salt, polymorph, prodrug, solvate or clathrate thereof.

Embodiment 19 relates to the compound of Embodiment 15, wherein the compounds of the formula (III) are compounds of the formulae:

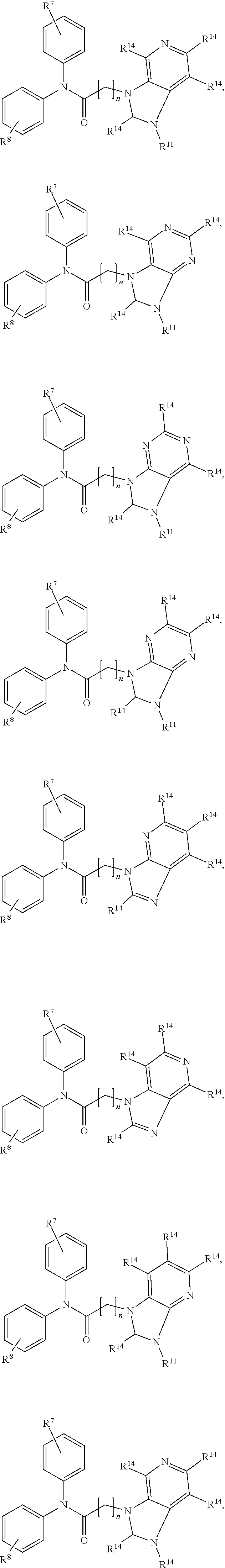

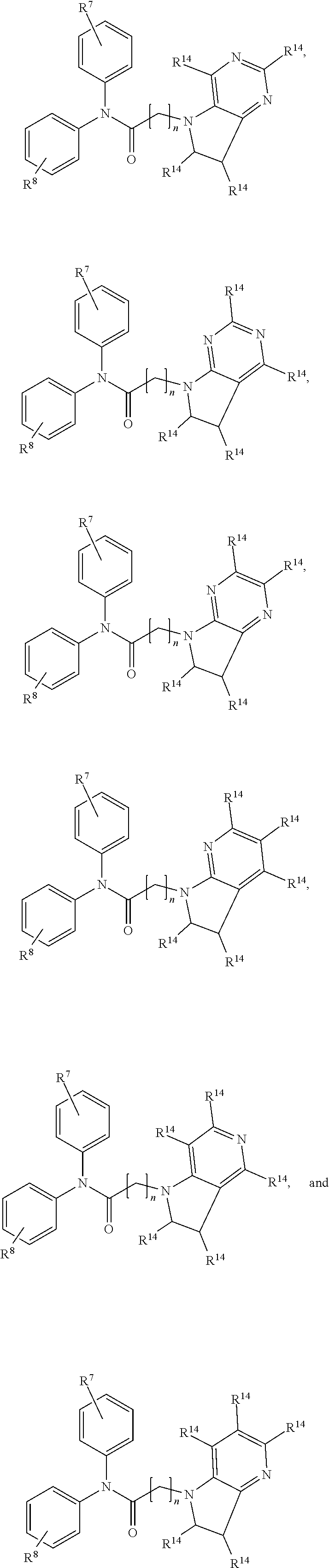

or a pharmaceutically acceptable salt, polymorph, prodrug, solvate or clathrate thereof.

Embodiment 20 relates to the compound of Embodiment 15, wherein the compounds of the formula (III) are compounds of the formulae:

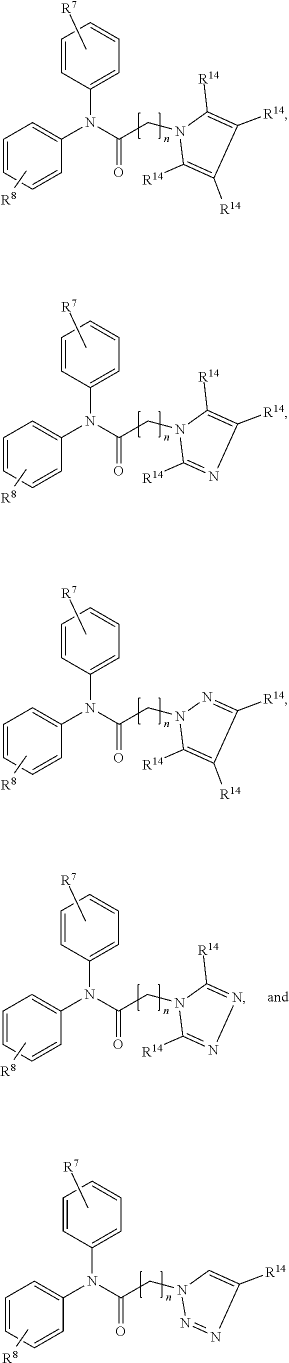

or a pharmaceutically acceptable salt, polymorph, prodrug, solvate or clathrate thereof

Embodiment 21 relates to a compound of the formula (IV):

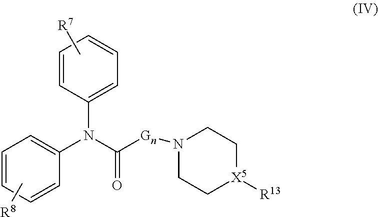

or a pharmaceutically acceptable salt, polymorph, prodrug, solvate or clathrate thereof,
wherein:
n is 0, 1 or 2;
each G is independently alkyl or C(O);
X⁵ is N or CR⁵, wherein R⁵ is hydrogen, alkyl, heterocyclyl or aryl;
R⁷ and R⁸ are each independently halo, a carbon with at least one halo (e.g., one to three halo, such as CHF₂, CCF₃, CCl₃), alkyl, aryl, acyl or heterocyclyl; and
R¹³ is a heterocyclyl group of the formula:

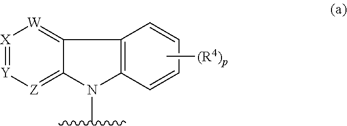

wherein W is N or C—R¹⁴; X is N or C—R¹⁴; Y is N or C—R¹⁴; and Z is N or C—R¹⁴; wherein R¹⁴ is hydrogen, an electron withdrawing group, alkyl, cycloakyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R⁴ is alkyl, cycloalkyl, aryl, heteroaryl, halo, amino, OR⁷, wherein R⁷ is hydrogen, alkyl, cycloalkyl, aryl or arylakyl, S(O)ₓ, wherein x is 0, 1 or 2, acyl, amido or heterocyclyl; and p is 0, 1, 2, 3 or 4;

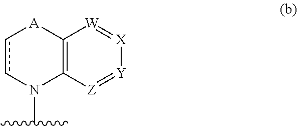

wherein A is S(O)ₓ, wherein x is 0, 1 or 2; O; C—R¹⁴, wherein R¹⁴ is hydrogen, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or A is N—R¹¹, wherein R¹¹ is a carbon with at least one halo, alkyl, aryl, acyl or heterocyclyl;

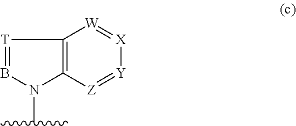

wherein T is CR¹⁴, wherein each R¹⁴ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or T is N; and
B is C—R¹⁴, wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or B is N;

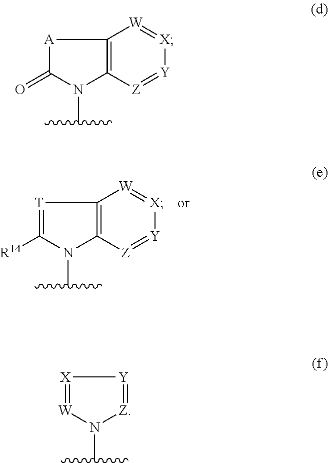

Embodiment 22 relates to the compound of Embodiment 21, wherein the compounds of the formula (IV) are compounds of the formulae:

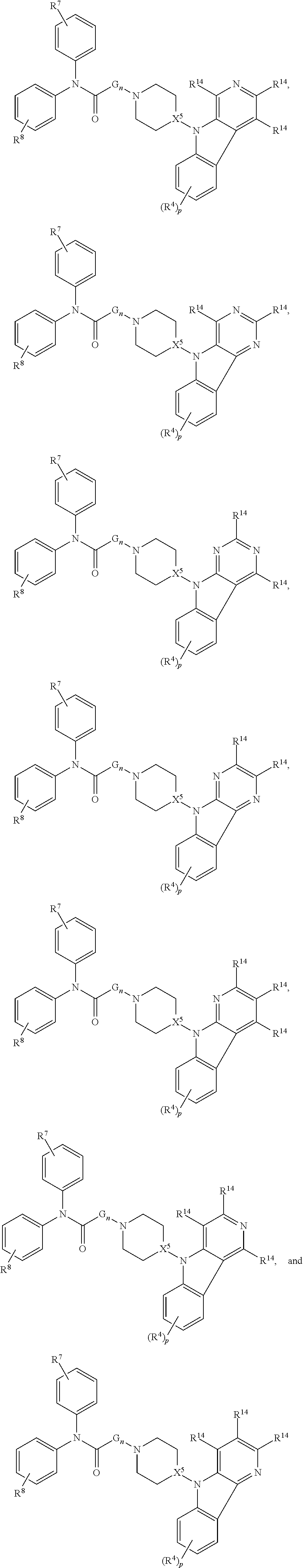

or a pharmaceutically acceptable salt, polymorph, prodrug, solvate or clathrate thereof.

Embodiment 23 relates to the compound of Embodiment 21, wherein the compounds of the formula (IV) are compounds of the formulae:

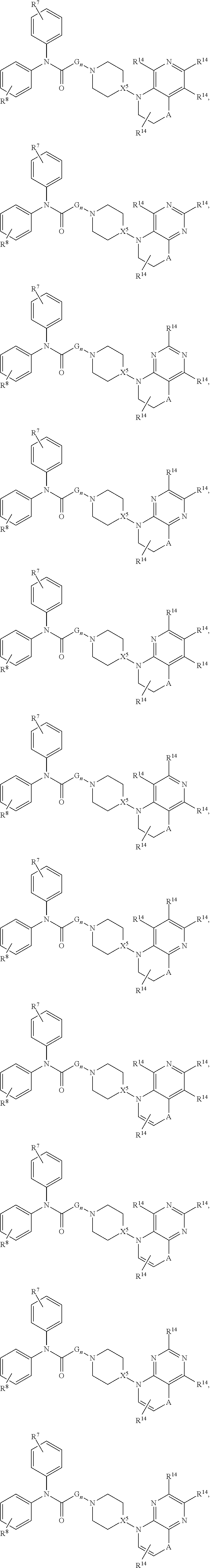

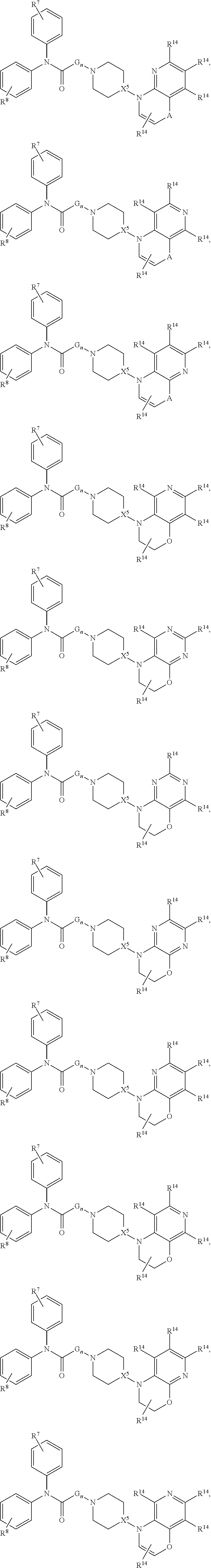

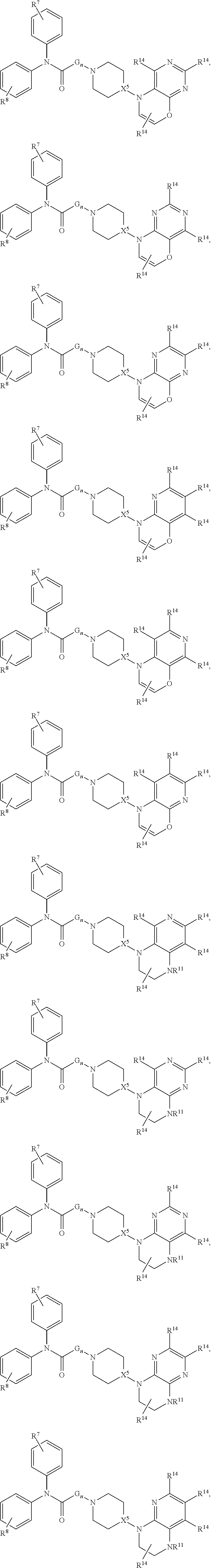

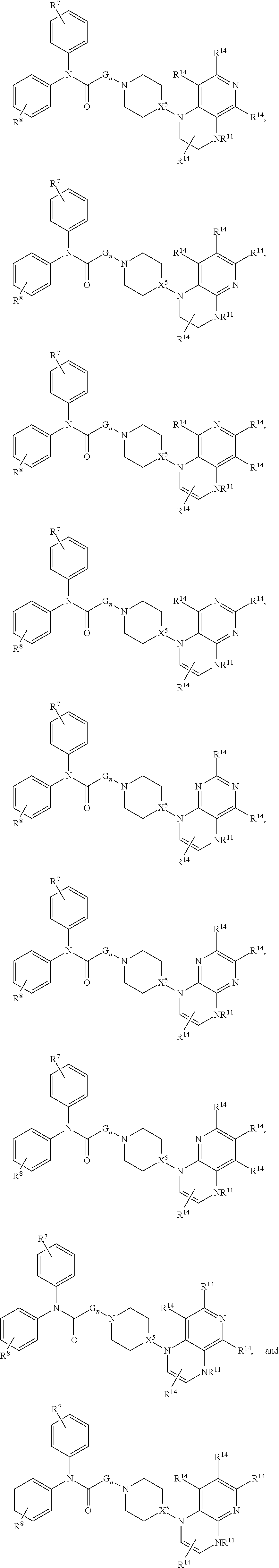

a pharmaceutically acceptable salt, polymorph, prodrug, solvate or clathrate thereof.

Embodiment 24 relates to the compound of Embodiment 21, wherein the compounds of the formula (IV) are compounds of the formulae:

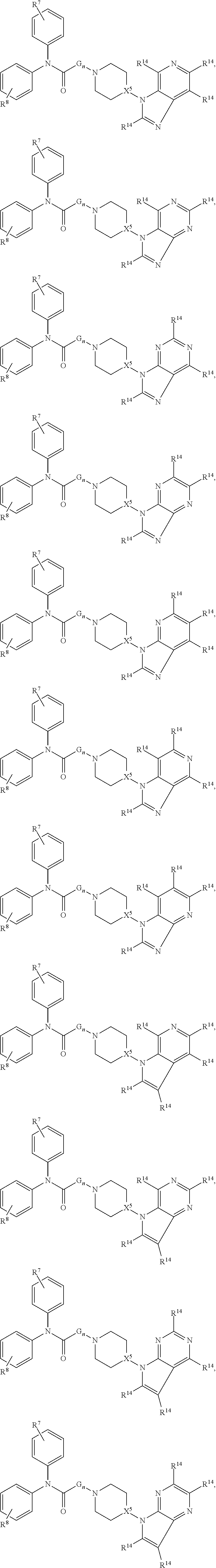

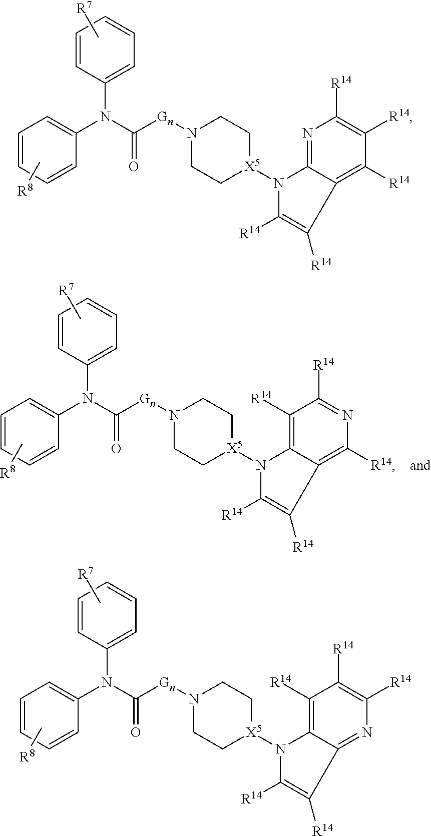

or a pharmaceutically acceptable salt, polymorph, prodrug, solvate or clathrate thereof.

Embodiment 25 relates to the compound of Embodiment 21, wherein the compounds of the formula (IV) are compounds of the formulae:

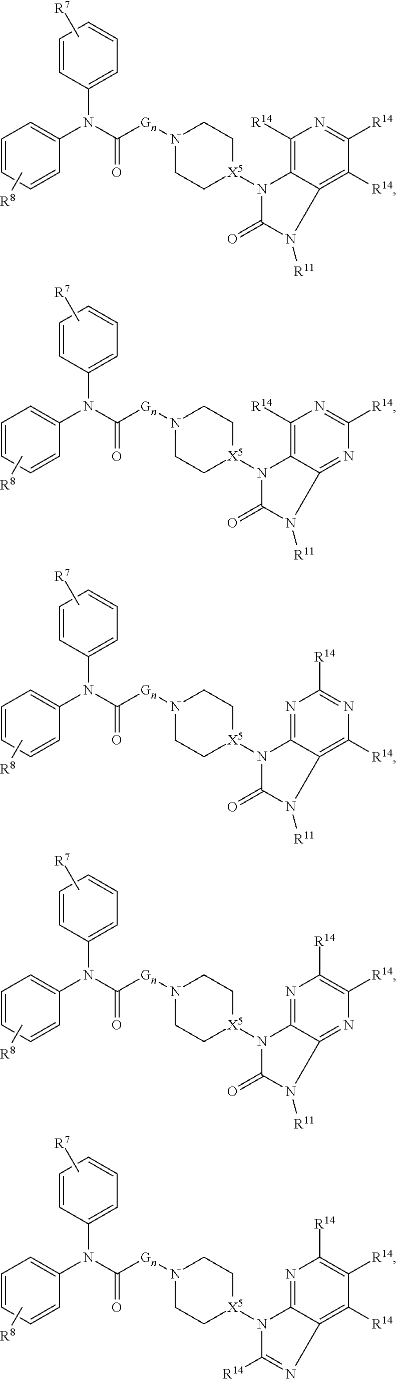

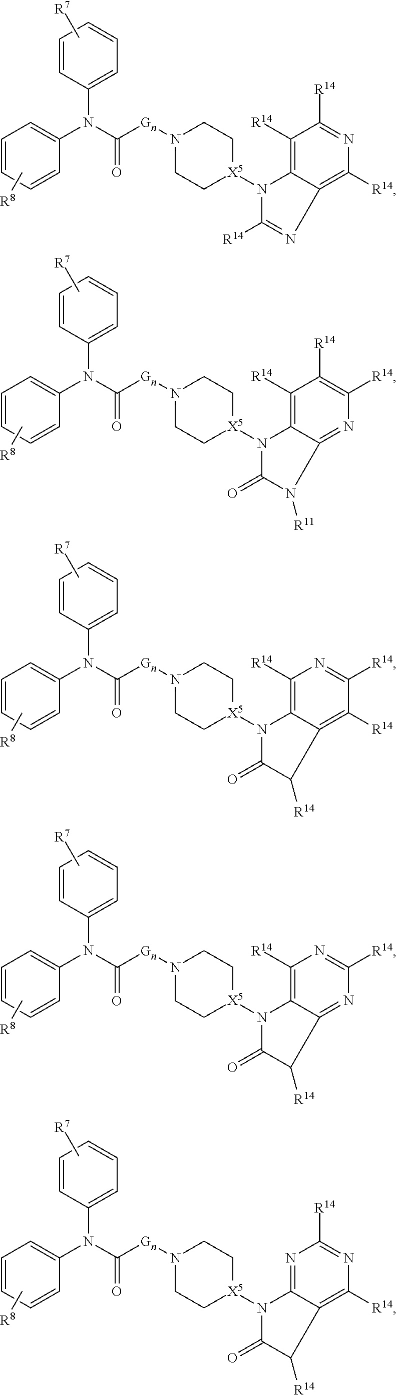

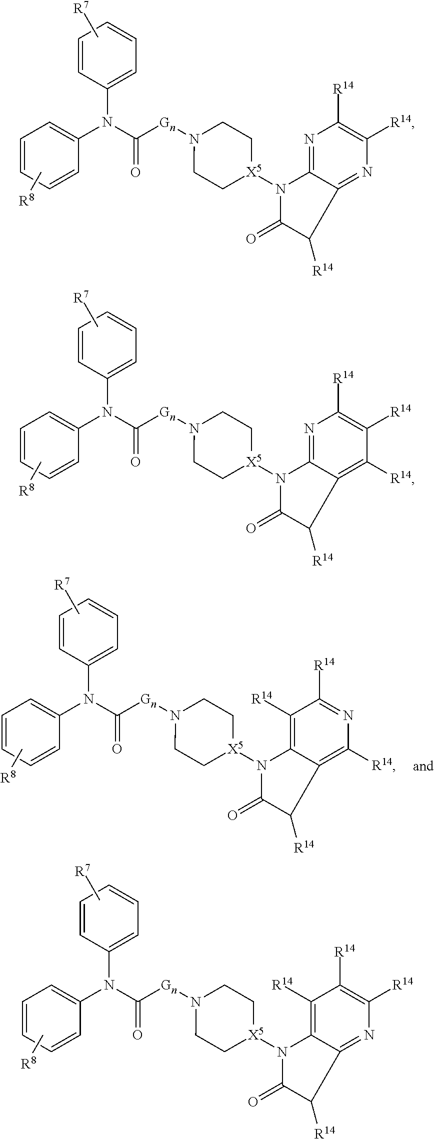

or a pharmaceutically acceptable salt, polymorph, prodrug, solvate or clathrate thereof.

Embodiment 26 relates to the compound of Embodiment 21, wherein the compounds of the formula (IV) are compounds of the formulae:

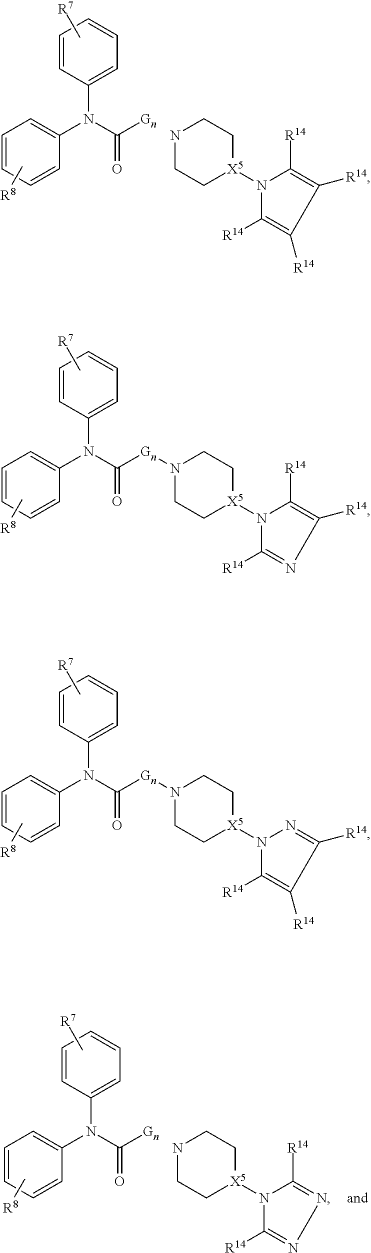

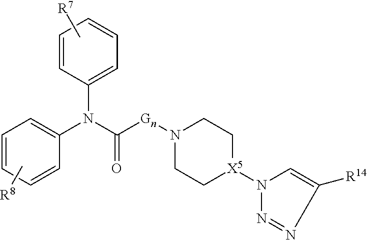

or a pharmaceutically acceptable salt, polymorph, prodrug, solvate or clathrate thereof.

Embodiment 27 relates to a compound of the formula (V):

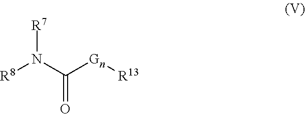

or a pharmaceutically acceptable salt, polymorph, prodrug, solvate or clathrate thereof,
wherein:
n is 0, 1 or 2;
each G is independently alkyl or C(O);
R⁷ and R⁸ are each independently halo, a carbon with at least one halo, alkyl, aryl, acyl or heterocyclyl; or
R⁷ and R⁸, together with the nitrogen atom to which they are attached, can form a heterocyclyl; and
R¹³ is a heterocyclyl group of the formula:

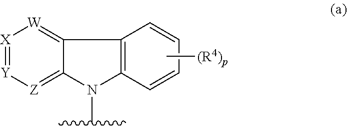

wherein W is N or C—R¹⁴; X is N or C—R¹⁴; Y is N or C—R¹⁴; and Z is N or C—R¹⁴; wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R⁴ is alkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4;

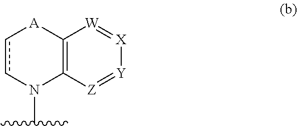

wherein A is S(O)ₓ, wherein x is 0, 1 or 2; O; C—R¹⁴, wherein R¹⁴ is hydrogen, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or A is N—R¹¹, wherein R¹¹ is a carbon with at least one halo, alkyl, aryl, acyl or heterocyclyl;

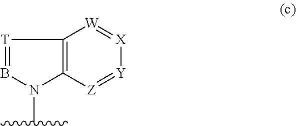

wherein T is CR¹⁴, wherein each R¹⁴ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or T is N; and
B is C—R¹⁴, wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or B is N;

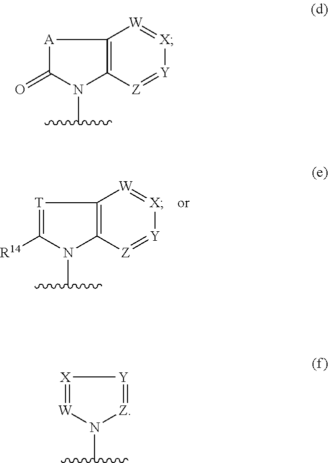

Embodiment 28 relates to the compound of Embodiment 27, wherein the compounds of the formula (V) are compounds of the formulae:

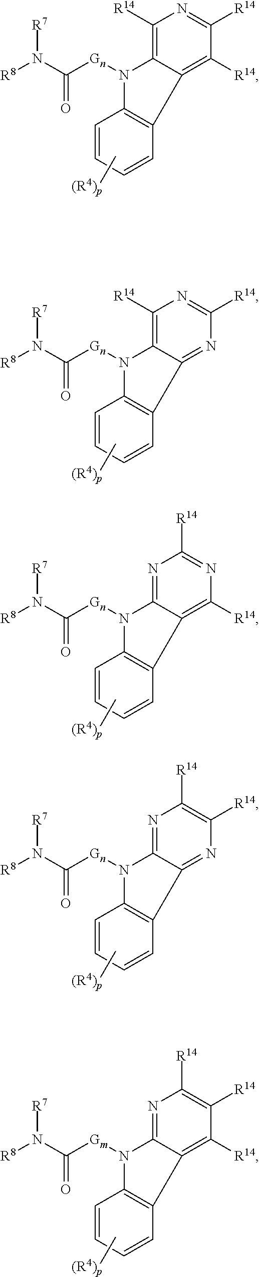

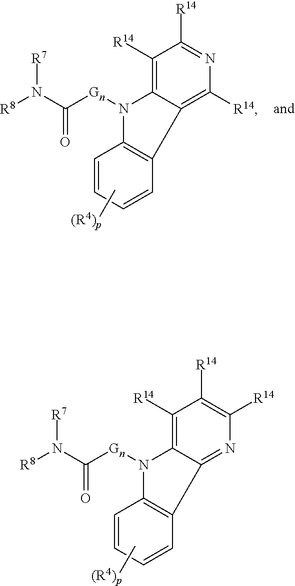

or a pharmaceutically acceptable salt, polymorph, prodrug, solvate or clathrate thereof.

Embodiment 29 relates to the compound of Embodiment 27, wherein the compounds of the formula (V) are compounds of the formulae:

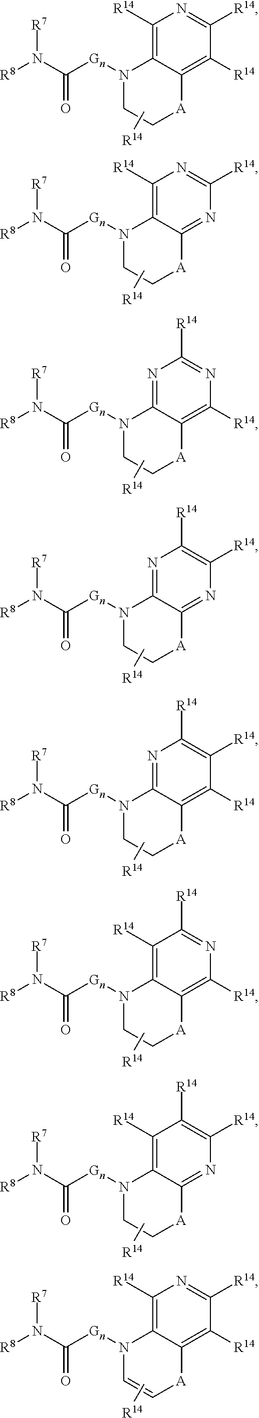

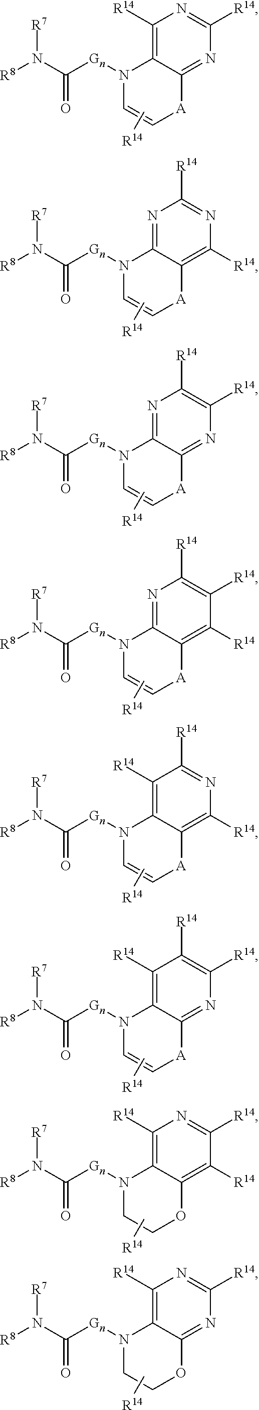

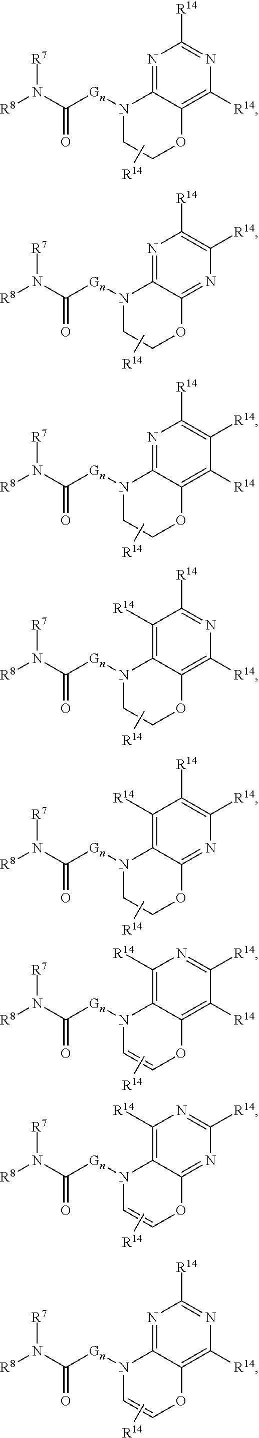

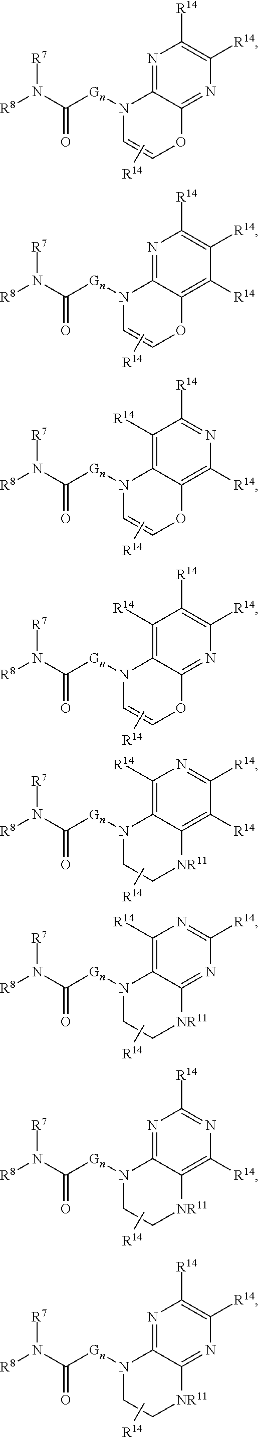

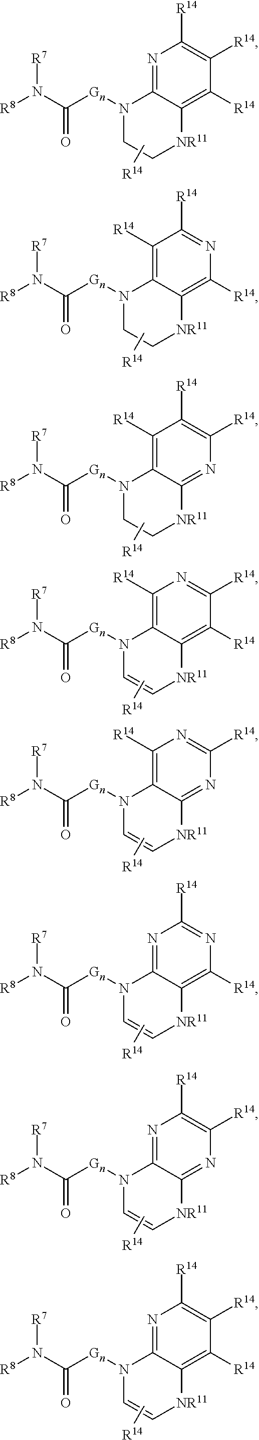

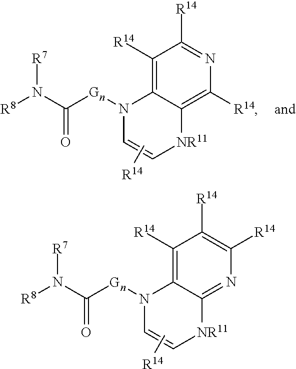

or a pharmaceutically acceptable salt, polymorph, prodrug, solvate or clathrate thereof.

Embodiment 30 relates to the compound of Embodiment 27, wherein the compounds of the formula (V) are compounds of the formulae:

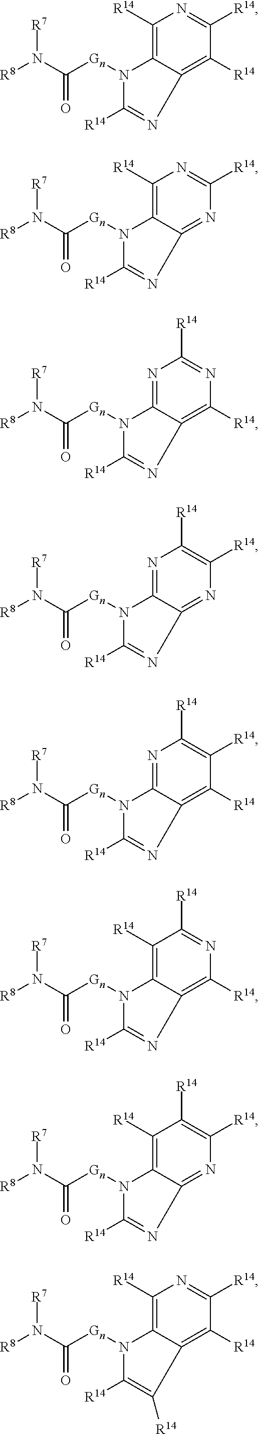

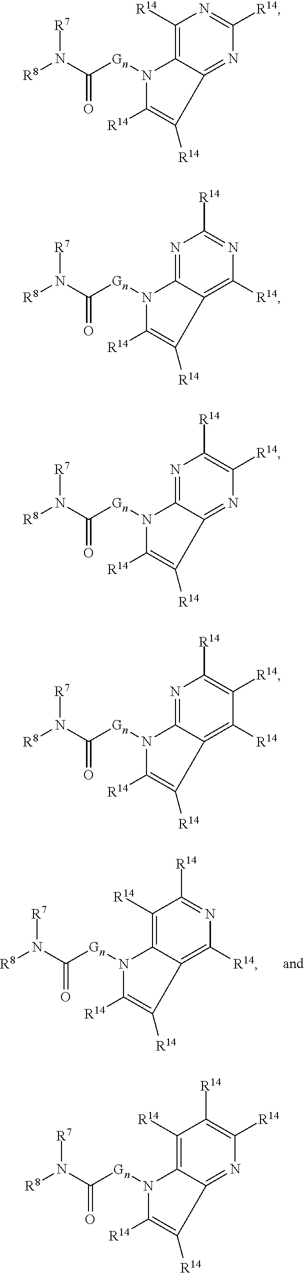

or a pharmaceutically acceptable salt, polymorph, prodrug, solvate or clathrate thereof.

Embodiment 31 relates to the compound of Embodiment 27, wherein the compounds of the formula (V) are compounds of the formulae:

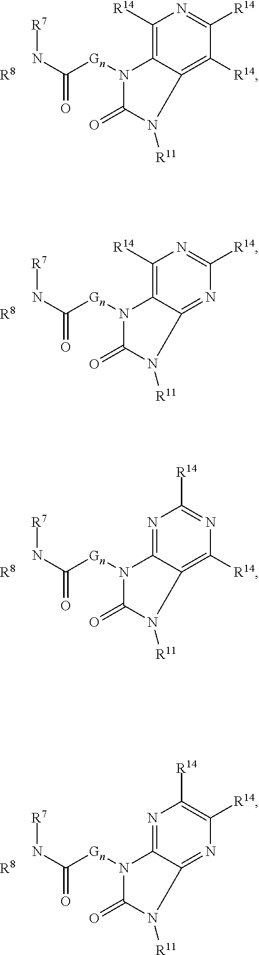

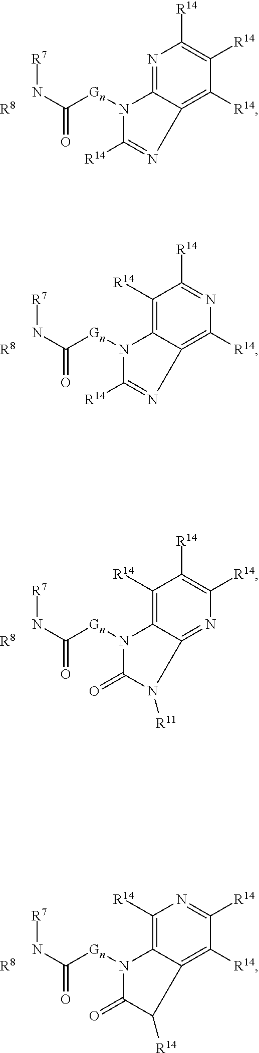

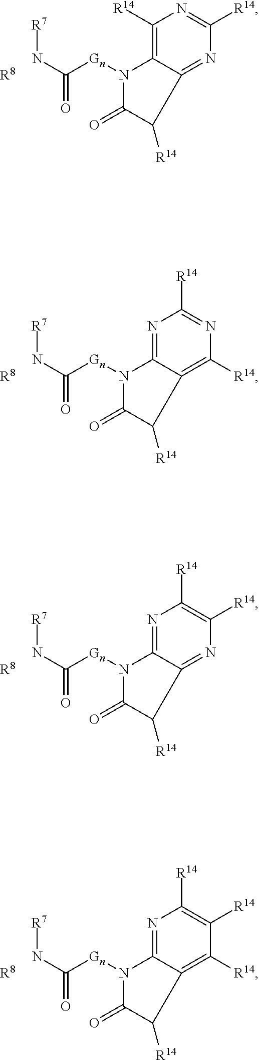

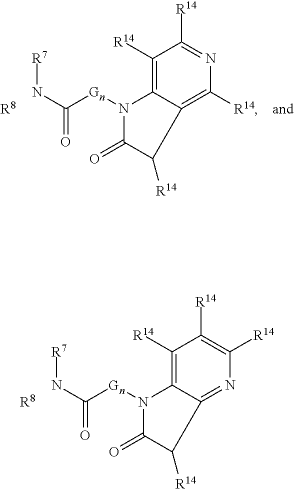

or a pharmaceutically acceptable salt, polymorph, prodrug, solvate or clathrate thereof.

Embodiment 32 relates to the compound of Embodiment 27, wherein the compounds of the formula (V) are compounds of the formulae:

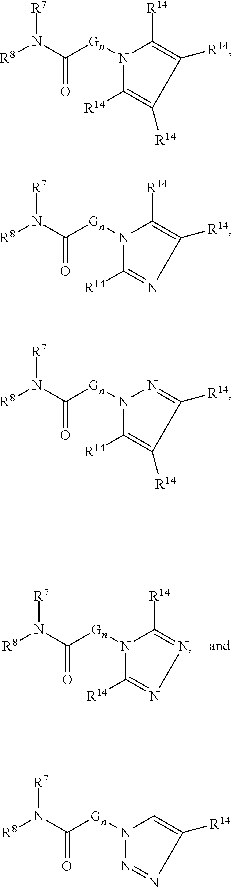

or a pharmaceutically acceptable salt, polymorph, prodrug, solvate or clathrate thereof.

Embodiment 33 relates to a compound of the formula (VI):

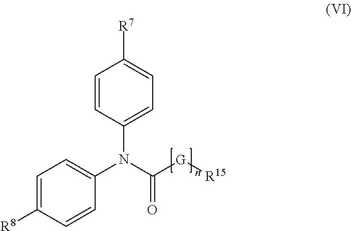

or a pharmaceutically acceptable salt, polymorph, prodrug, solvate or clathrate thereof,
wherein:
n is 0, 1 or 2;
each G is independently alkyl or C(O); and
R⁷ and R⁸ are each independently halo, a carbon with at least one halo, alkyl, aryl, acyl or heterocyclyl; and
R¹⁵ is a heterocyclyl group of the formula:

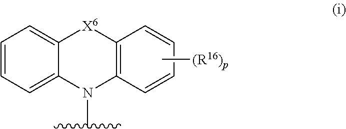

wherein X⁶ is alkyl (e.g., CH₂ and CH₂CH₂), alkenyl (e.g., CH═CH), S, O or NR¹⁷; wherein R¹⁷ is hydrogen, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R¹⁶ is alkyl, haloalkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4;

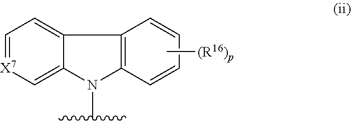

wherein X⁷ is N or C—R¹⁸; wherein R¹⁸ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and
R¹⁶ is alkyl, haloalkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4;

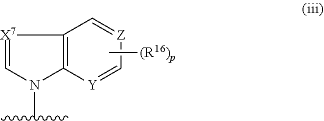

wherein X⁷ is N or C—R¹⁸; wherein R¹⁸ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R¹⁶ is alkyl, haloalkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4;
Y is N or C—R¹⁴; and Z is N or C—R¹⁴; wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;

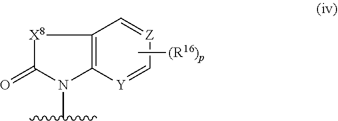

wherein X⁸ is NR¹⁹, wherein R¹⁹ is H, alkyl or aryl or X⁸ is C(R¹⁸)₂; wherein each R¹⁸ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R¹⁶ is alkyl, haloalkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4;
Y is N or C—R¹⁴; and Z is N or C—R¹⁴; wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;

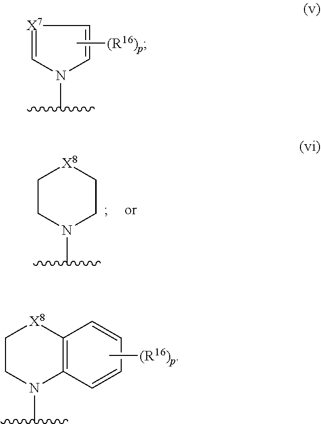

Embodiment 34 relates to the compound of Embodiment 33, wherein the compound is a compound of the formula:

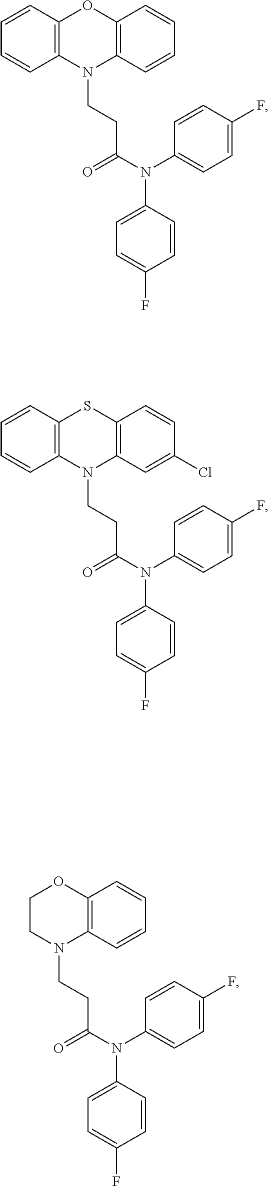 [SMILES: O=C(CCN1c2ccccc2Oc2ccccc21)N(c1ccc(F)cc1)c1ccc(F)cc1 ; O=C(CCN1c2ccccc2Sc2ccc(Cl)cc21)N(c1ccc(F)cc1)c1ccc(F)cc1 ; O=C(CCN1CCOc2ccccc21)N(c1ccc(F)cc1)c1ccc(F)cc1]

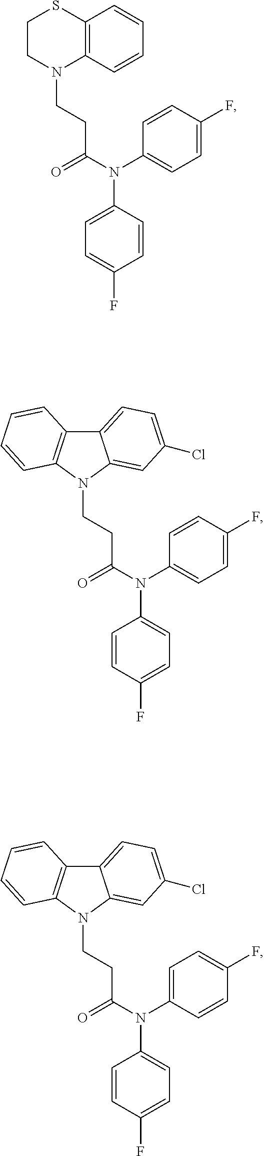 [SMILES: O=C(CCN1CCSc2ccccc21)N(c1ccc(F)cc1)c1ccc(F)cc1 ; O=C(CCn1c2ccccc2c2ccc(Cl)cc21)N(c1ccc(F)cc1)c1ccc(F)cc1 ; O=C(CCn1c2ccccc2c2ccc(Cl)cc21)N(c1ccc(F)cc1)c1ccc(F)cc1]

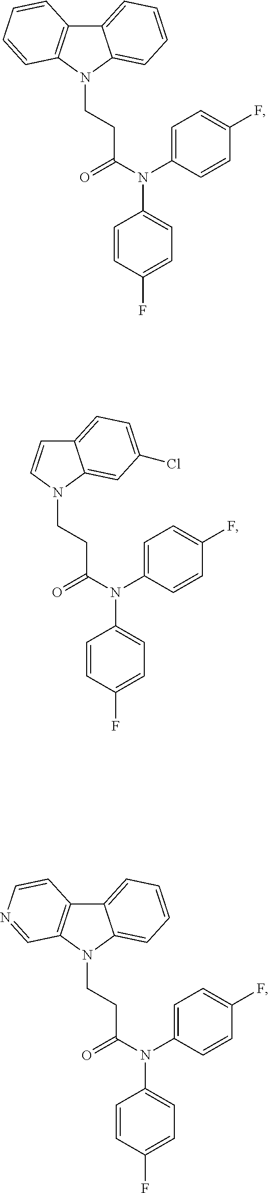 [SMILES: O=C(CCn1c2ccccc2c2ccccc21)N(c1ccc(F)cc1)c1ccc(F)cc1 ; O=C(CCn1ccc2ccc(Cl)cc21)N(c1ccc(F)cc1)c1ccc(F)cc1 ; O=C(CCn1c2ccccc2c2ccncc21)N(c1ccc(F)cc1)c1ccc(F)cc1]

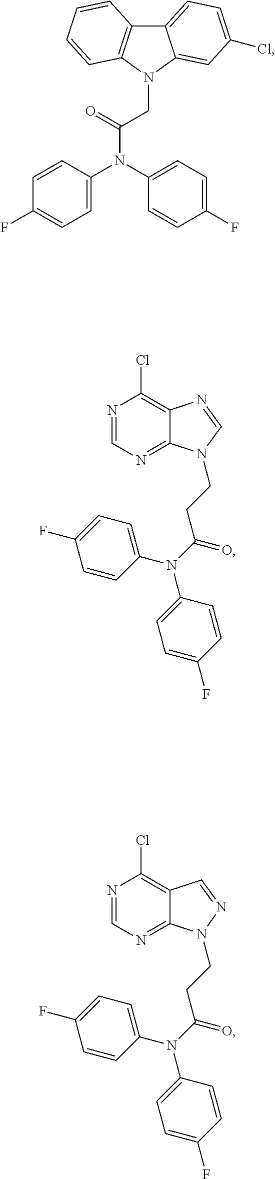 [SMILES: O=C(Cn1c2ccccc2c2ccc(Cl)cc21)N(c1ccc(F)cc1)c1ccc(F)cc1 ; O=C(CCn1cnc2c(Cl)ncnc21)N(c1ccc(F)cc1)c1ccc(F)cc1 ; O=C(CCn1ncc2c(Cl)ncnc21)N(c1ccc(F)cc1)c1ccc(F)cc1]

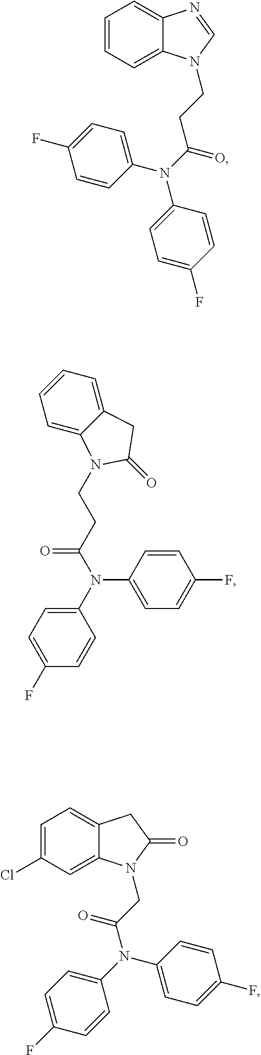 [SMILES: O=C(CCn1cnc2ccccc21)N(c1ccc(F)cc1)c1ccc(F)cc1 ; O=C1Cc2ccccc2N1CCC(=O)N(c1ccc(F)cc1)c1ccc(F)cc1 ; O=C1Cc2ccc(Cl)cc2N1CC(=O)N(c1ccc(F)cc1)c1ccc(F)cc1]

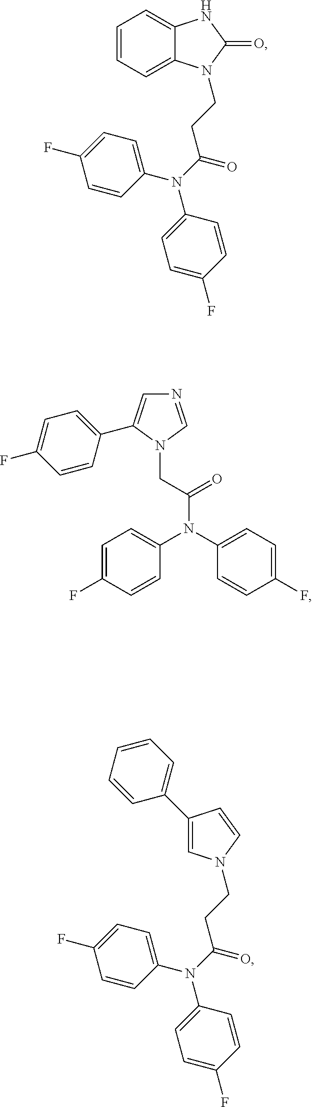 [SMILES: O=C(CCn1c(=O)[nH]c2ccccc21)N(c1ccc(F)cc1)c1ccc(F)cc1 ; O=C(Cn1cncc1-c1ccc(F)cc1)N(c1ccc(F)cc1)c1ccc(F)cc1 ; O=C(CCn1ccc(-c2ccccc2)c1)N(c1ccc(F)cc1)c1ccc(F)cc1]

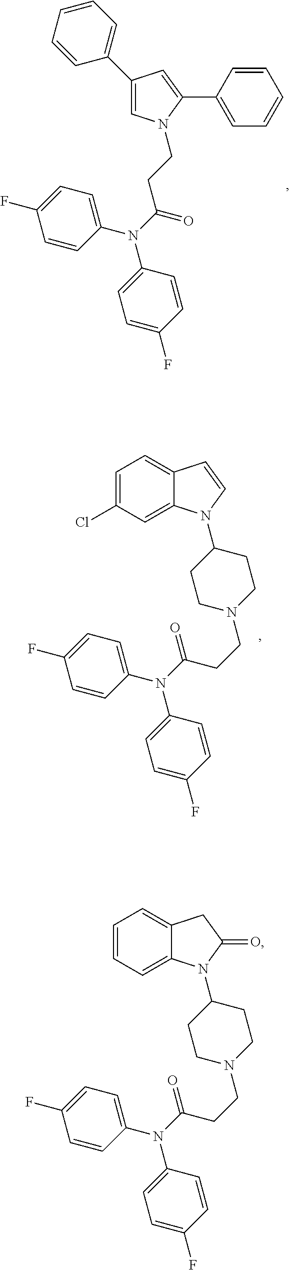 [SMILES: O=C(CCn1cc(-c2ccccc2)cc1-c1ccccc1)N(c1ccc(F)cc1)c1ccc(F)cc1 ; O=C(CCN1CCC(n2ccc3ccc(Cl)cc32)CC1)N(c1ccc(F)cc1)c1ccc(F)cc1 ; O=C(CCN1CCC(N2C(=O)Cc3ccccc32)CC1)N(c1ccc(F)cc1)c1ccc(F)cc1]

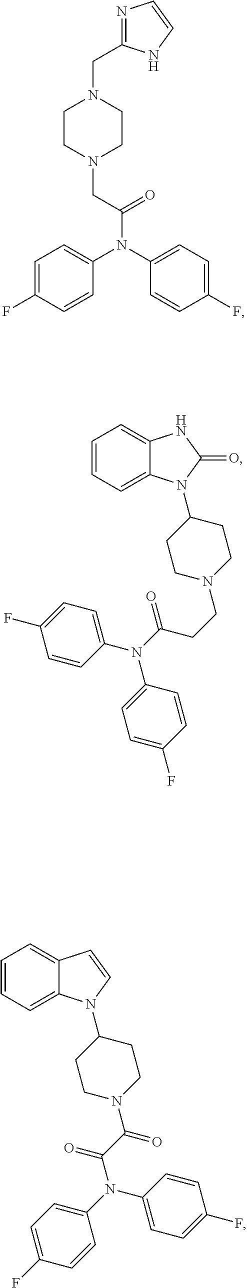 [SMILES: O=C(CN1CCN(Cc2ncc[nH]2)CC1)N(c1ccc(F)cc1)c1ccc(F)cc1 ; O=C(CCN1CCC(n2c(=O)[nH]c3ccccc32)CC1)N(c1ccc(F)cc1)c1ccc(F)cc1 ; O=C(C(=O)N(c1ccc(F)cc1)c1ccc(F)cc1)N1CCC(n2ccc3ccccc32)CC1]

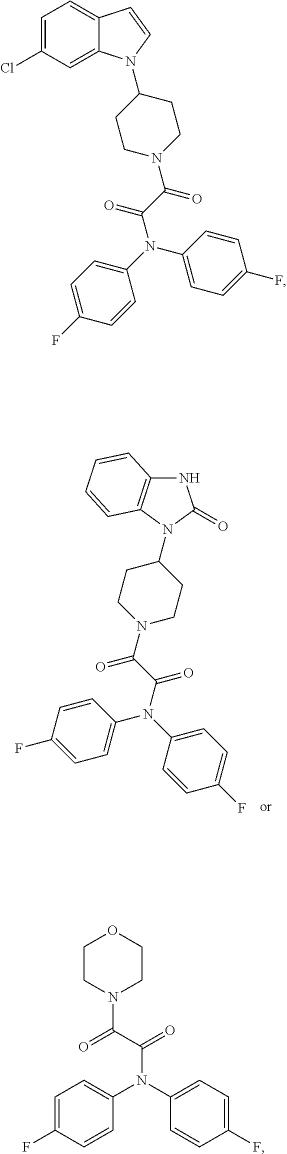 [SMILES: O=C(C(=O)N(c1ccc(F)cc1)c1ccc(F)cc1)N1CCC(n2ccc3ccc(Cl)cc32)CC1 ; O=C(C(=O)N(c1ccc(F)cc1)c1ccc(F)cc1)N1CCC(n2c(=O)[nH]c3ccccc32)CC1 ; O=C(C(=O)N(c1ccc(F)cc1)c1ccc(F)cc1)N1CCOCC1]

or a pharmaceutically acceptable salt, polymorph, prodrug, solvate or clathrate thereof.

Embodiment 35 relates to a pharmaceutical composition comprising one or more compounds of any preceding claim and one or more pharmaceutically acceptable excipients.

Embodiment 36 relates to a method for treating a neurodegenerative disease comprising administering a therapeutically effective amount of chlorpromazine, at least one compound of Embodiments 1-34 or a pharmaceutical composition of Embodiment 35 to a subject in need thereof.

Embodiment 37 relates to the method of Embodiment 36, wherein the neurodegenerative disease is at least one of Parkinson's disease, Alzheimer's disease, Huntington's disease, and ALS.

Embodiment 38 relates to a method for reducing, substantially eliminating or eliminating dysregulation of proteostasis comprising administering a therapeutically effective amount of chlorpromazine, at least one compound of Embodiments 1-34 or a pharmaceutical composition of Embodiment 35 to a subject in need thereof.

Embodiment 39 relates to a method for reducing, substantially eliminating or eliminating the accumulation of intrinsically disordered proteins comprising administering a therapeutically effective amount of chlorpromazine, at least one compound of Embodiments 1-34 or a pharmaceutical composition of Embodiment 35 to a subject in need thereof.

1.-50. (canceled)

51. A compound of the formula (I): 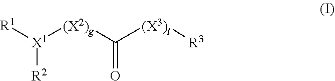
or a pharmaceutically acceptable salt, polymorph, prodrug, solvate or clathrate thereof, wherein:
R¹ is hydrogen, alkyl, cycloalkyl, aryl, heterocyclyl or heteroaryl;
R² is hydrogen, alkyl, cycloalkyl, aryl, heterocyclyl or heteroaryl;
R³ is aryl or heteroaryl;
X¹ is N or CR⁵, wherein R⁵ is absent, hydrogen, alkyl, heterocyclyl or aryl;
X² is alkyl or alkenyl;
X³ is alkyl or alkenyl;
g is 0 or 1; and
t is 0 or 1;
provided that g and t are not simultaneously 0. In some examples, g is 0 and t is 1. In other examples, g is 1 and t is 0.

52. The compound of claim 51, wherein the compound of the formula (I) is a compound of the formula (Ia): 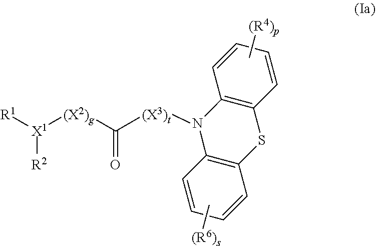
or a pharmaceutically acceptable salt, polymorph, prodrug, solvate or clathrate thereof, wherein:
R¹ is hydrogen, alkyl, cycloalkyl, aryl, heterocyclyl or heteroaryl;
R² is hydrogen, alkyl, cycloalkyl, aryl, heterocyclyl or heteroaryl;
R³ is aryl or heteroaryl;
X¹ is N or CR⁵, wherein R⁵ is absent, hydrogen, alkyl, heterocyclyl or aryl;
X² is alkyl or alkenyl;
X³ is alkyl or alkenyl;
g is 0 or 1; and
t is 0 or 1;
provided that g and t are not simultaneously 0;
R⁴ is alkyl, cycloalkyl, aryl, heteroaryl, halo, amino, OR⁷, wherein R¹ is hydrogen, alkyl, cycloalkyl, aryl or arylalkyl, S(O)ₓ, wherein x is 0, 1 or 2, acyl, amido or heterocyclyl;
R⁶ is alkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
p is 0, 1, 2, 3 or 4; and
s is 0, 1, 2, 3 or 4; or
a compound of the formula (II): 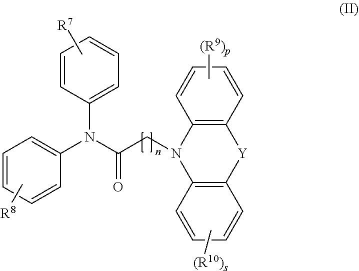
or a pharmaceutically acceptable salt, polymorph, prodrug, solvate or clathrate thereof,
wherein:
n is 0, 1 or 2;
Y is S(O)ₓ, wherein x is 0, 1 or 2, O, CH₂, or Y is N—R¹¹, wherein R¹¹ is a carbon with at least one halo, alkyl, aryl, acyl or heterocyclyl;
R⁷, R⁸, R⁹, and R¹⁰ are each independently halo, a carbon with at least one halo, alkyl, aryl, acyl or heterocyclyl;
p is 0, 1, 2, 3 or 4; and
s is 0, 1, 2, 3 or 4.

53. The compound of claim 51, wherein the compound of formula (I) is a compound of the formula (Ib), (Ic), (Id), (Ie), (If), (Ig), (Ih), (Ii), (Ij) or (Ik): 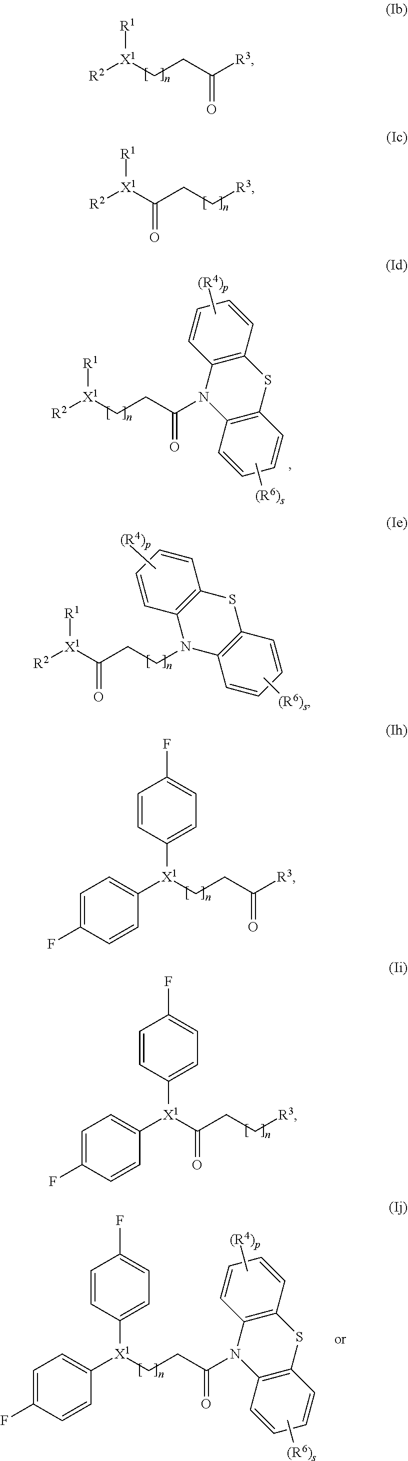 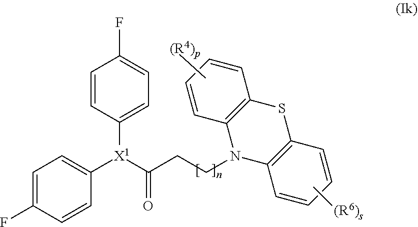
or a pharmaceutically acceptable salt, polymorph, prodrug, solvate or clathrate thereof,
wherein:
n is 0, 1 or 2; or 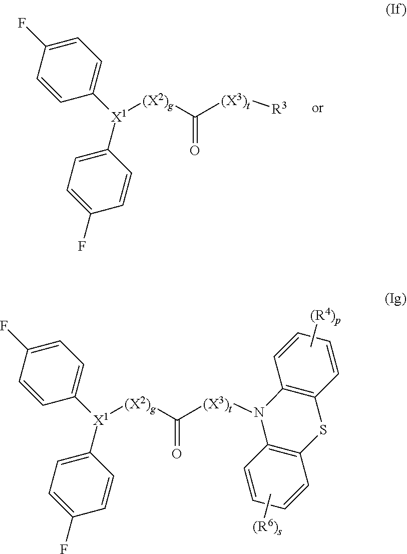
or a pharmaceutically acceptable salt, polymorph, prodrug, solvate or clathrate thereof, wherein:
R³ is aryl or heteroaryl;
X¹ is N or CR⁵, wherein R⁵ is absent, hydrogen, alkyl, heterocyclyl or aryl;
X² is alkyl or alkenyl;
X³ is alkyl or alkenyl;
g is 0 or 1; and
t is 0 or 1;
provided that g and t are not simultaneously 0.

54. The compound of claim 51, wherein the compound of the formula (I) is a compound of the formula: 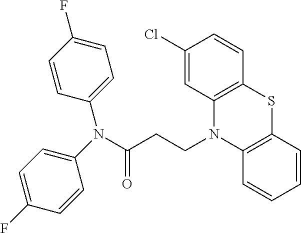 [SMILES: O=C(CCN1c2ccccc2Sc2ccc(Cl)cc21)N(c1ccc(F)cc1)c1ccc(F)cc1]
pharmaceutically acceptable salts, polymorphs, prodrugs, solvates or clathrates thereof.

55. A compound of the formula (III): 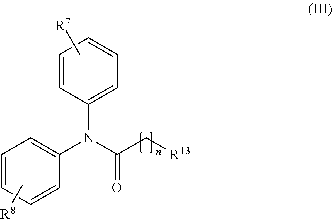
or a pharmaceutically acceptable salt, polymorph, prodrug, solvate or clathrate thereof,
wherein:
n is 0, 1 or 2;
R⁷ and R⁸ are each independently halo, a carbon with at least one halo, alkyl, aryl, acyl or heterocyclyl; and
R¹³ is a heterocyclyl group of the formula: 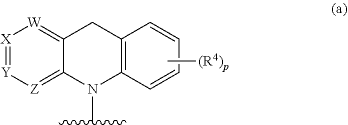
wherein W is N or C—R¹⁴; X is N or C—R¹⁴; Y is N or C—R¹⁴; and Z is N or C—R¹⁴; wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R⁴ is alkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4; 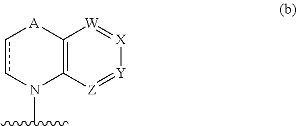
wherein the dashed line can represent a double bond; A is S(O)ₓ, wherein x is 0, 1 or 2; O; C(R¹⁴)₂, wherein each R¹⁴ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or A is N—R¹¹, wherein R¹¹ is a carbon with at least one halo, alkyl, aryl, acyl or heterocyclyl; 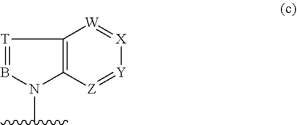
wherein T is CR¹⁴, wherein each R¹⁴ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or T is N; and
B is C—R¹⁴, wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or B is N;

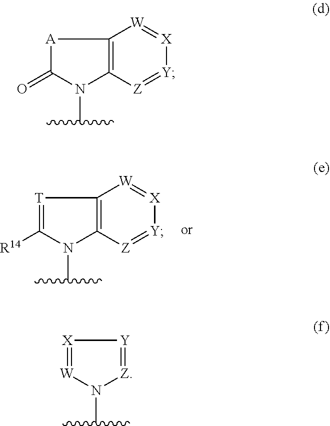

56. The compound of claim 55, wherein the compounds of the formula (III) are compounds of the formulae: 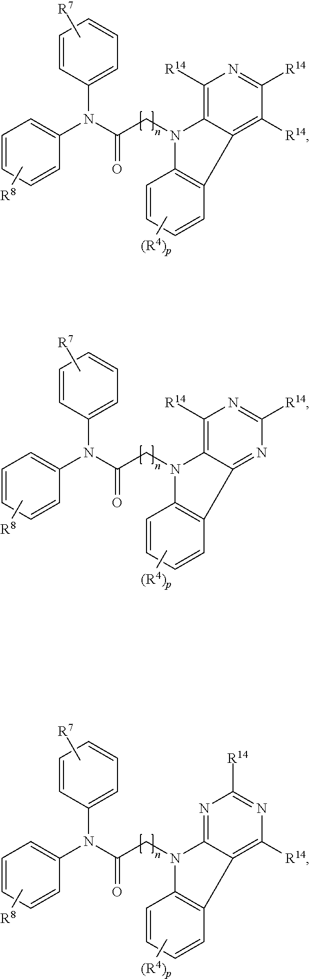 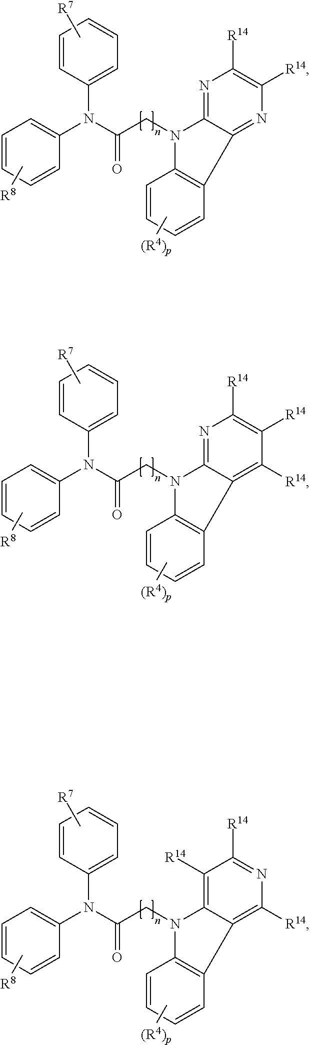 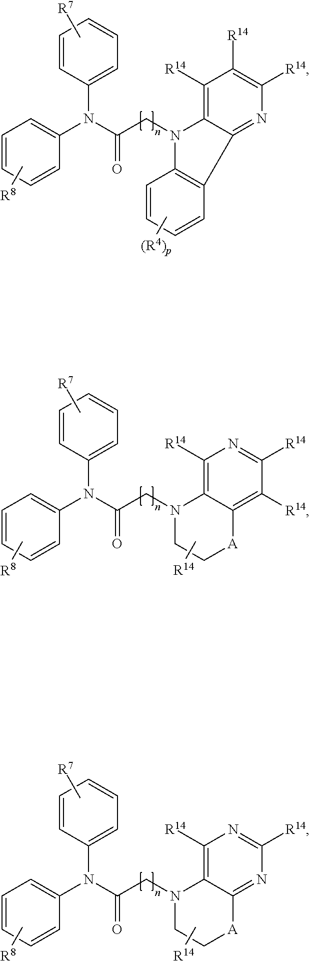 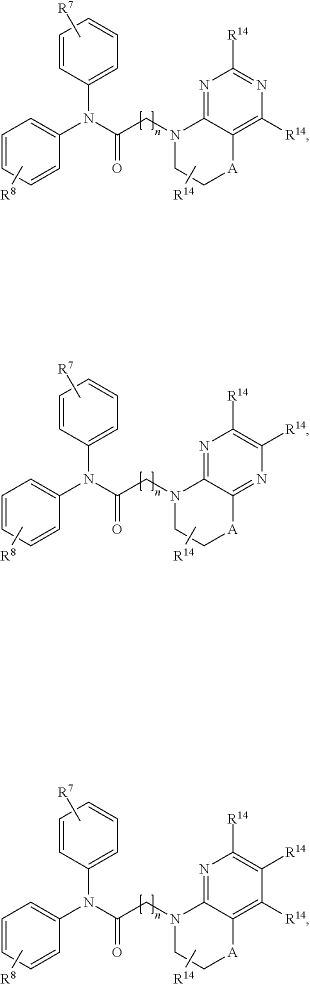 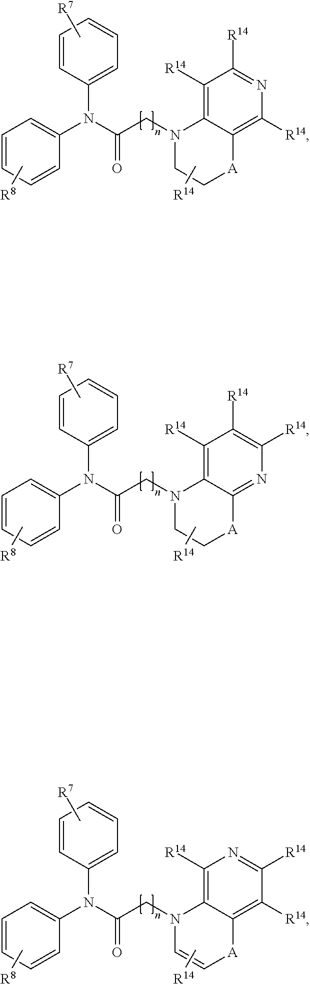 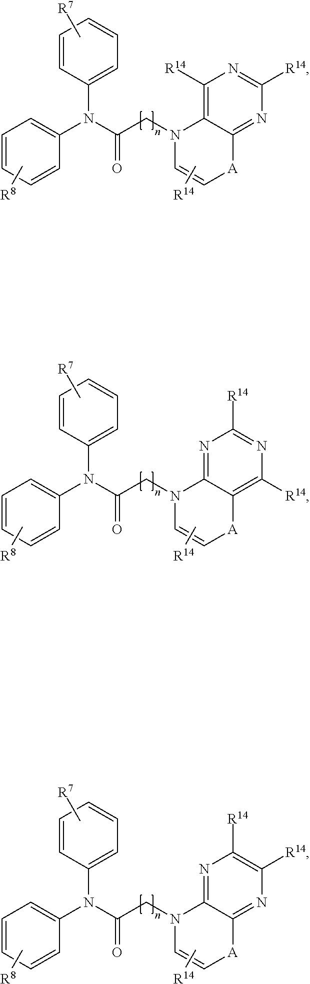 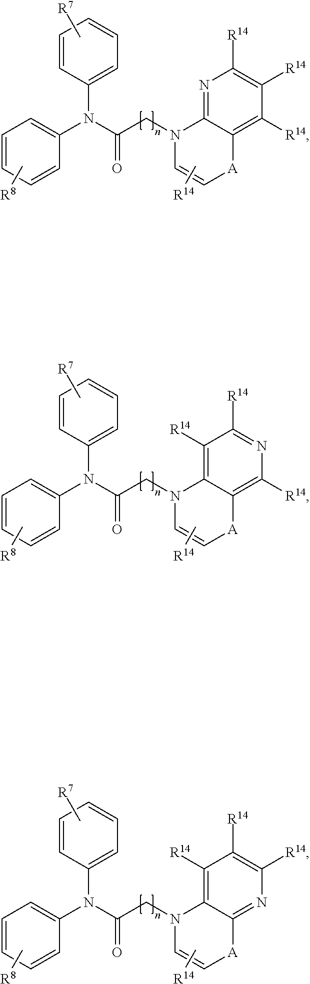 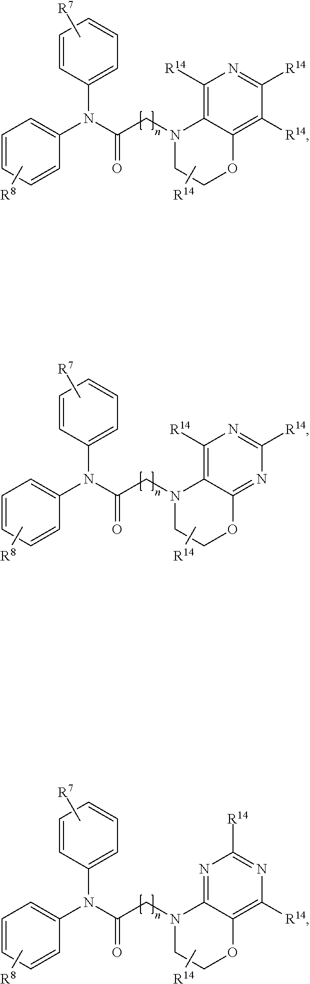 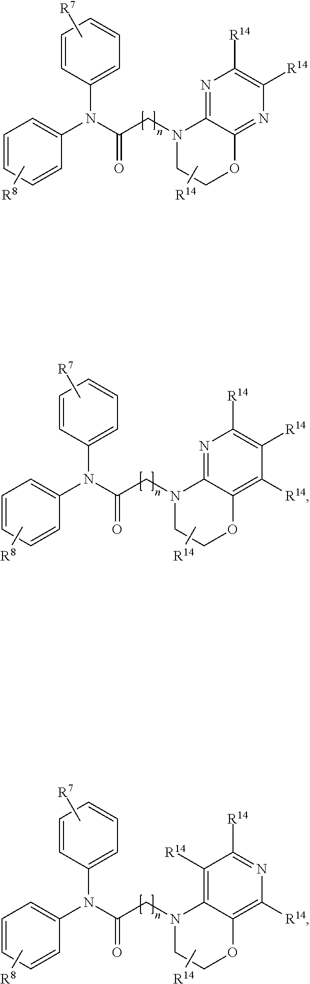 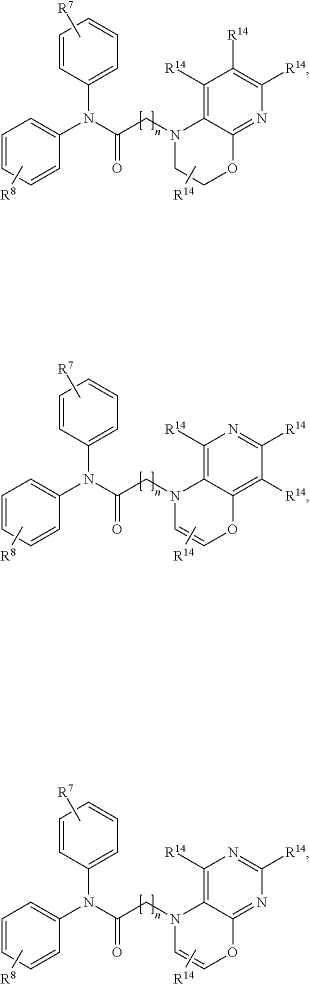 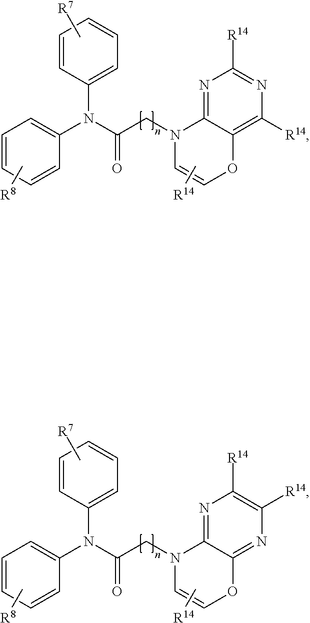 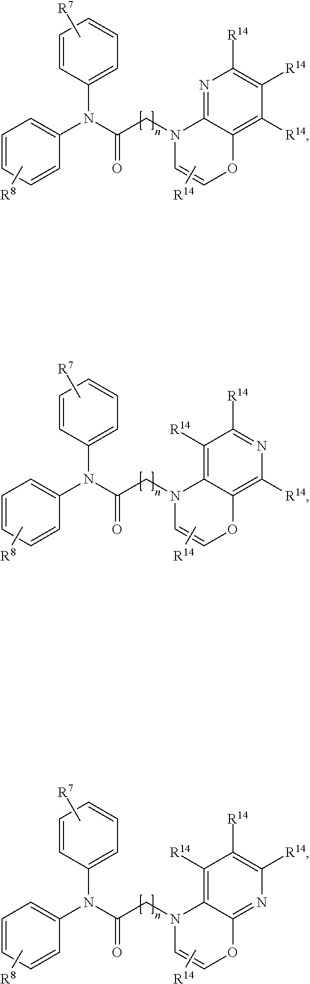 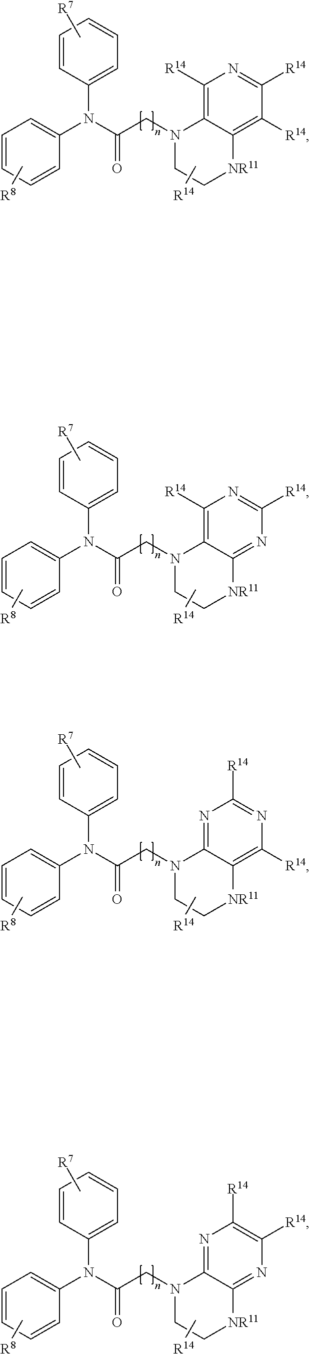 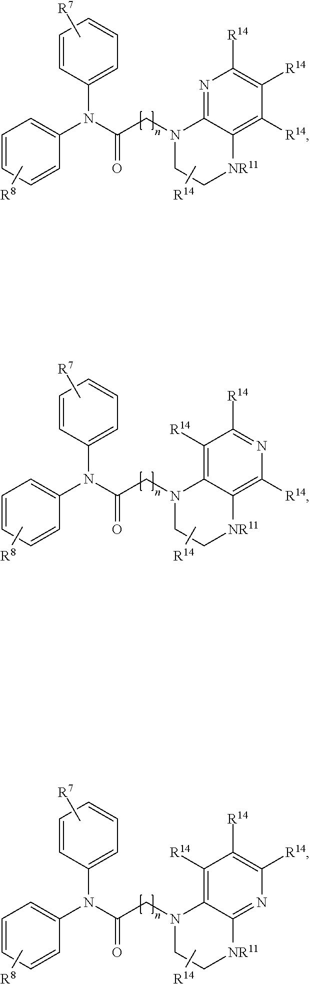 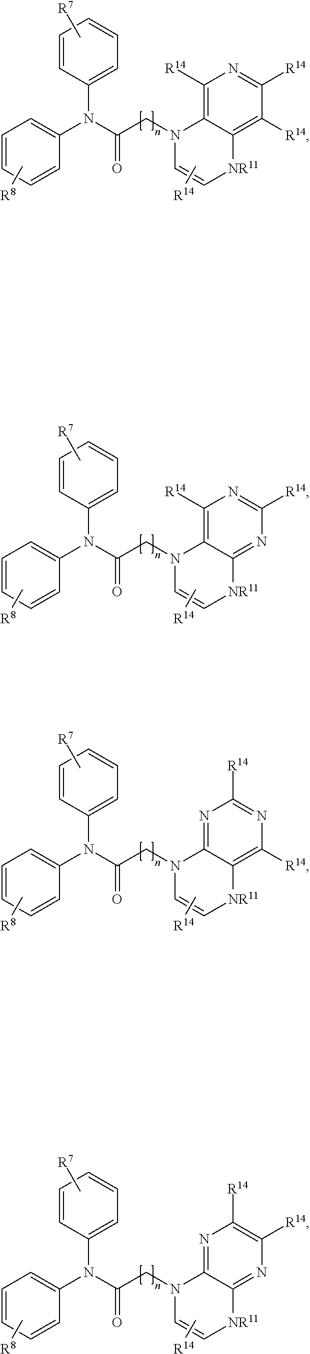 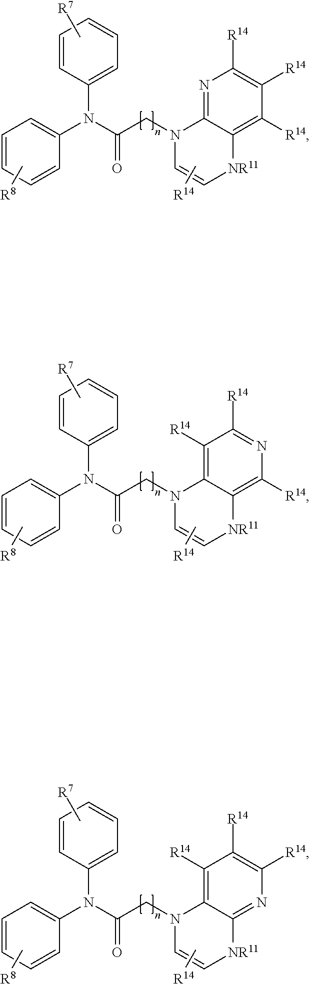 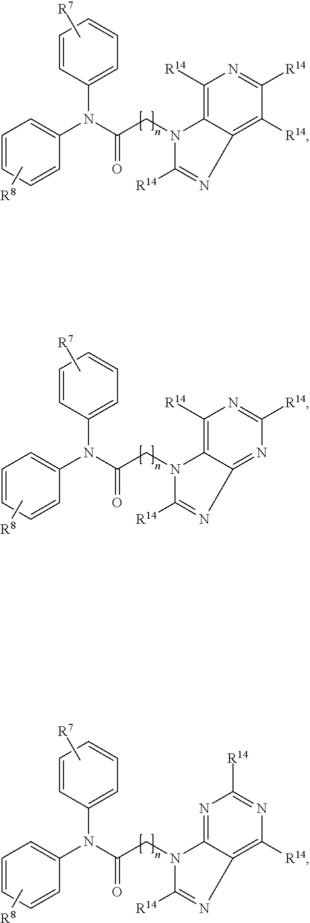 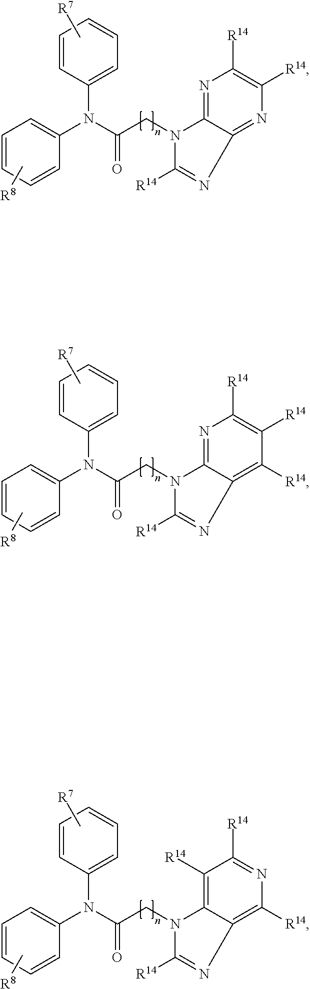 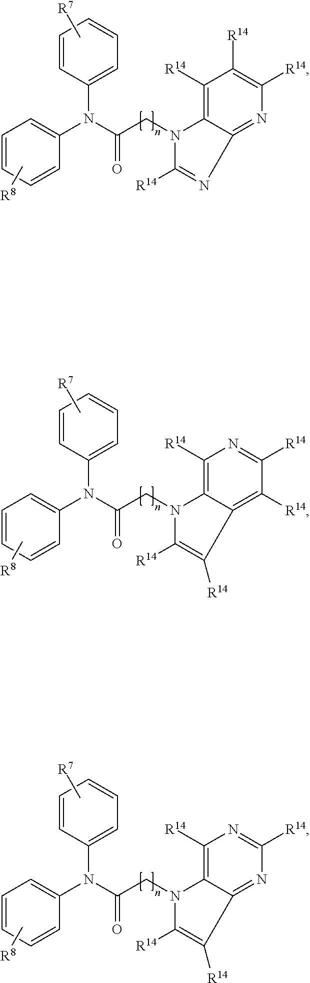 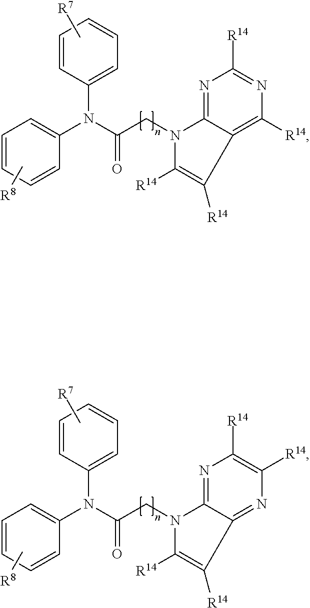 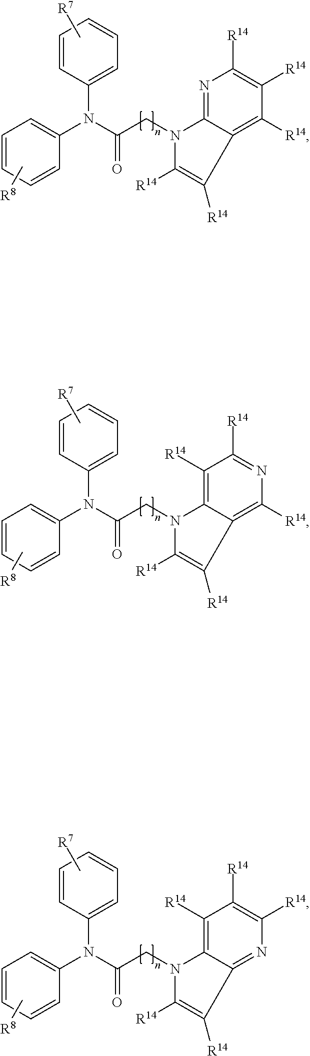 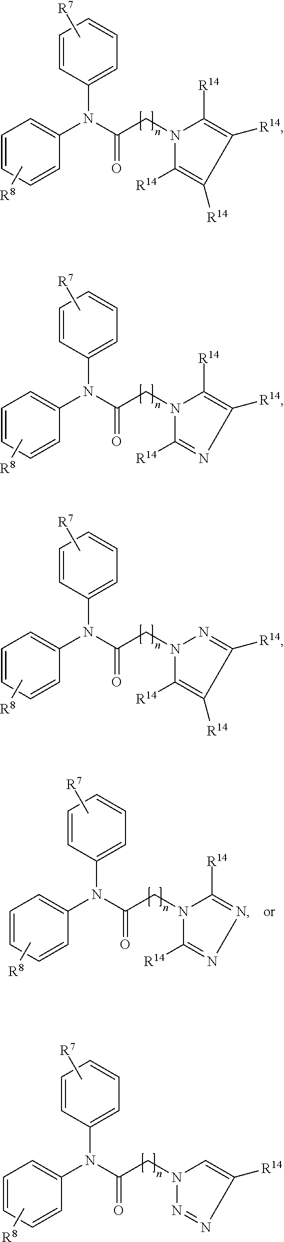
or a pharmaceutically acceptable salt, polymorph, prodrug, solvate or clathrate thereof.

57. A compound of the formula (IV): 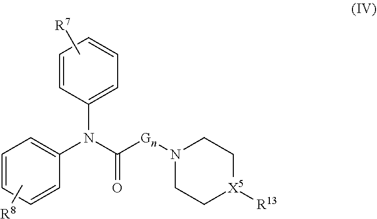
or a pharmaceutically acceptable salt, polymorph, prodrug, solvate or clathrate thereof,
wherein:
n is 0, 1 or 2;
each G is independently alkyl or C(O);
X⁵ is N or CR⁵, wherein R⁵ is hydrogen, alkyl, heterocyclyl or aryl;
R⁷ and R⁸ are each independently halo, a carbon with at least one halo (e.g., one to three halo, such as CHF₂, CCF₃, CCl₃), alkyl, aryl, acyl or heterocyclyl; and
R¹³ is a heterocyclyl group of the formula: 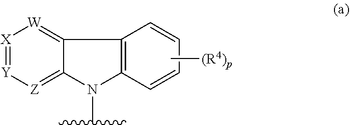
wherein W is N or C—R¹⁴; X is N or C—R¹⁴; Y is N or C—R¹⁴; and Z is N or C—R¹⁴; wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R⁴ is alkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4; 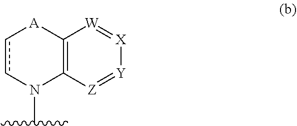
wherein A is S(O)ₓ, wherein x is 0, 1 or 2; O; C—R¹⁴, wherein R¹⁴ is hydrogen, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or A is N—R¹¹, wherein R¹¹ is a carbon with at least one halo, alkyl, aryl, acyl or heterocyclyl; 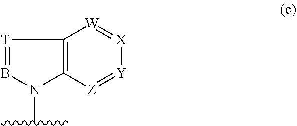
wherein T is CR¹⁴, wherein each R¹⁴ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or T is N; and
B is C—R¹⁴, wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or B is N;

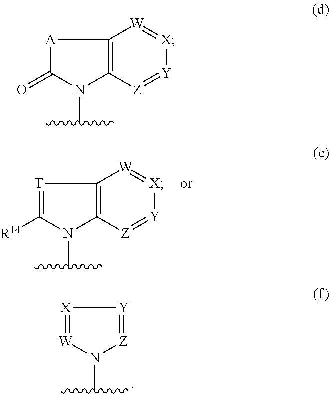

58. The compound of claim 57, wherein the compounds of the formula (IV) are compounds of the formulae: 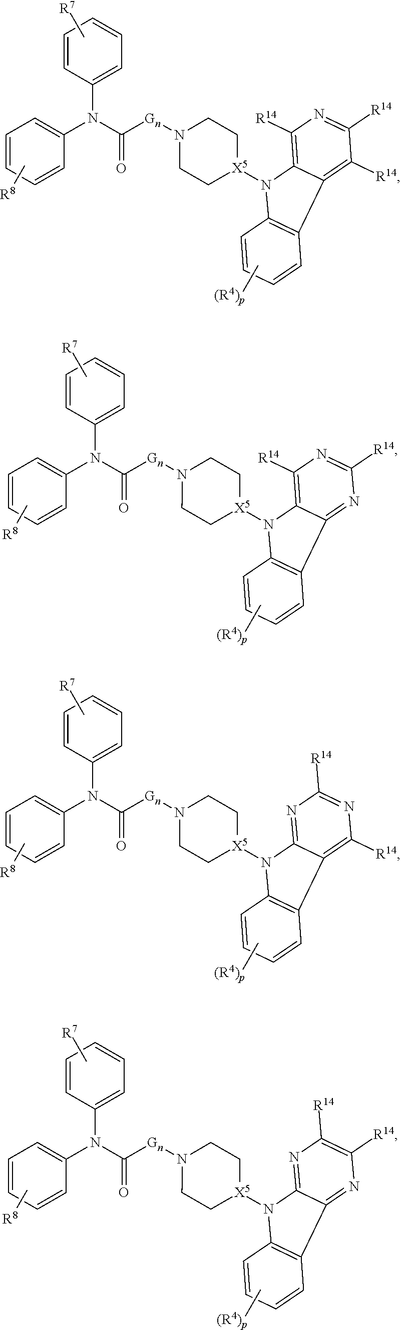 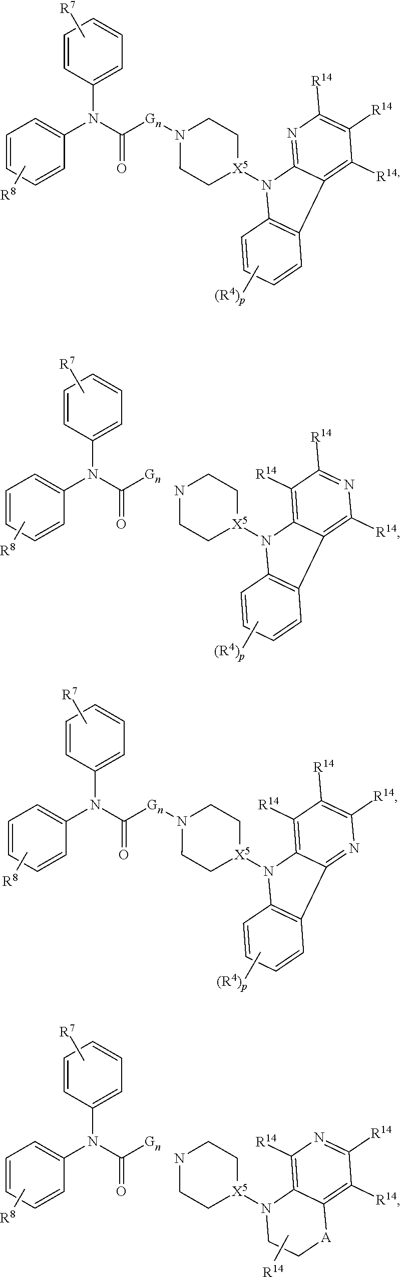 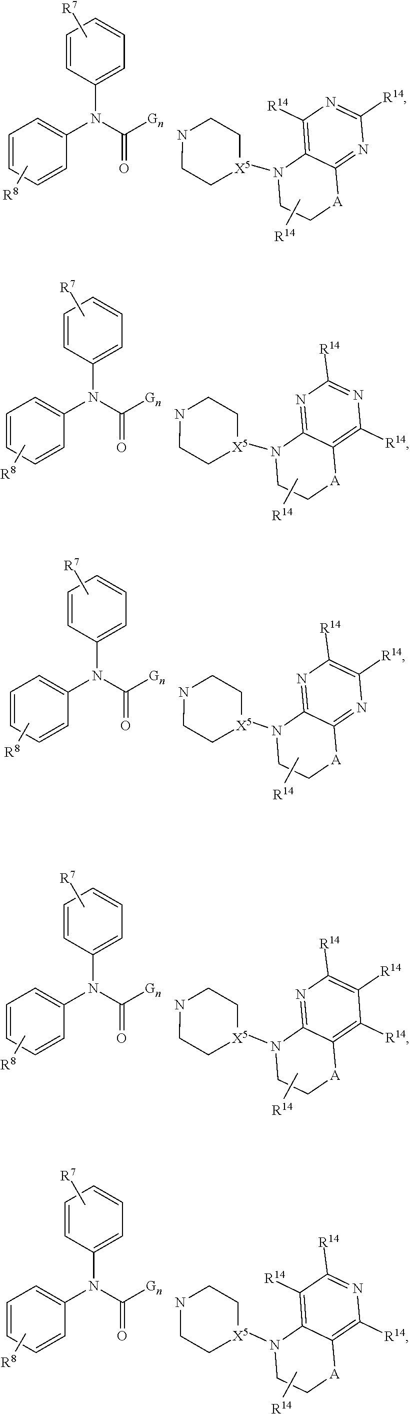 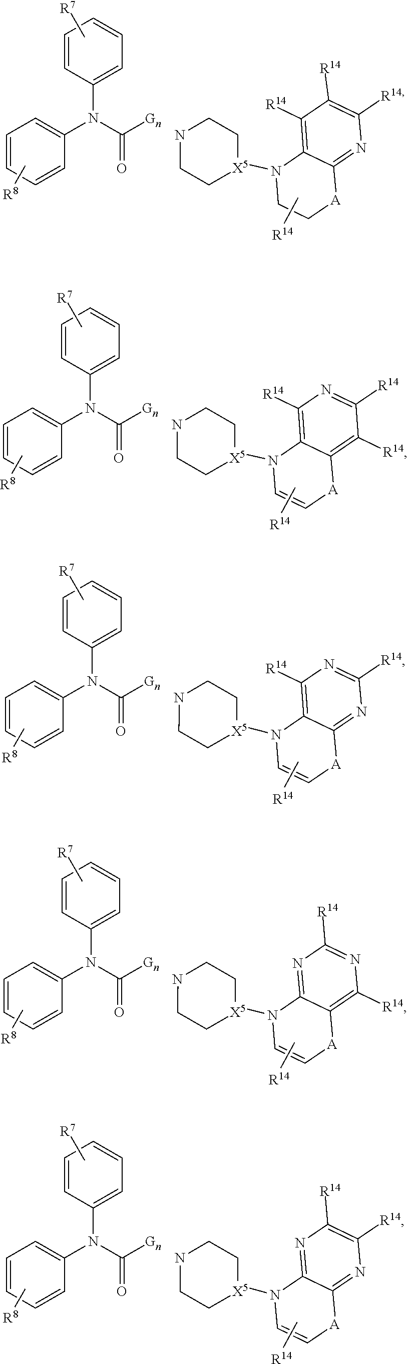 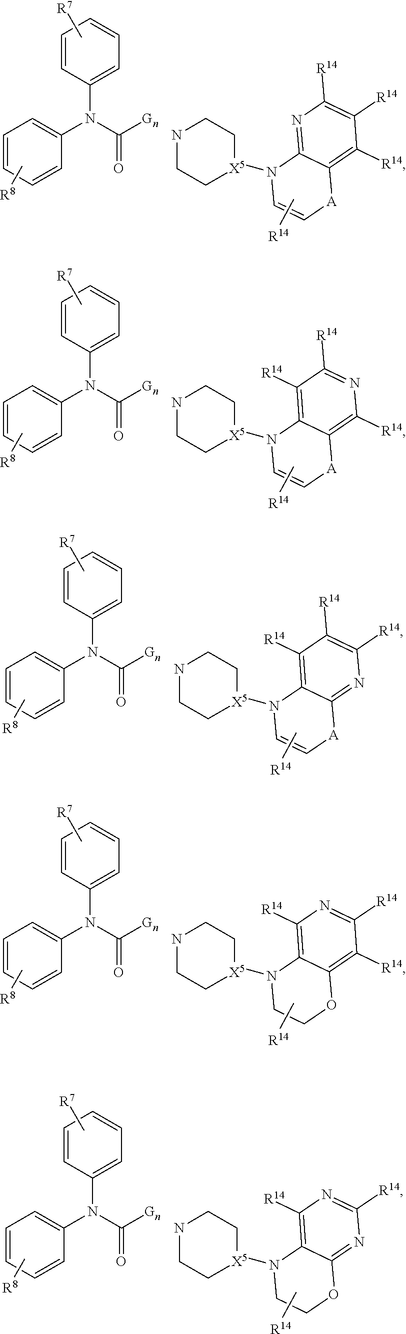 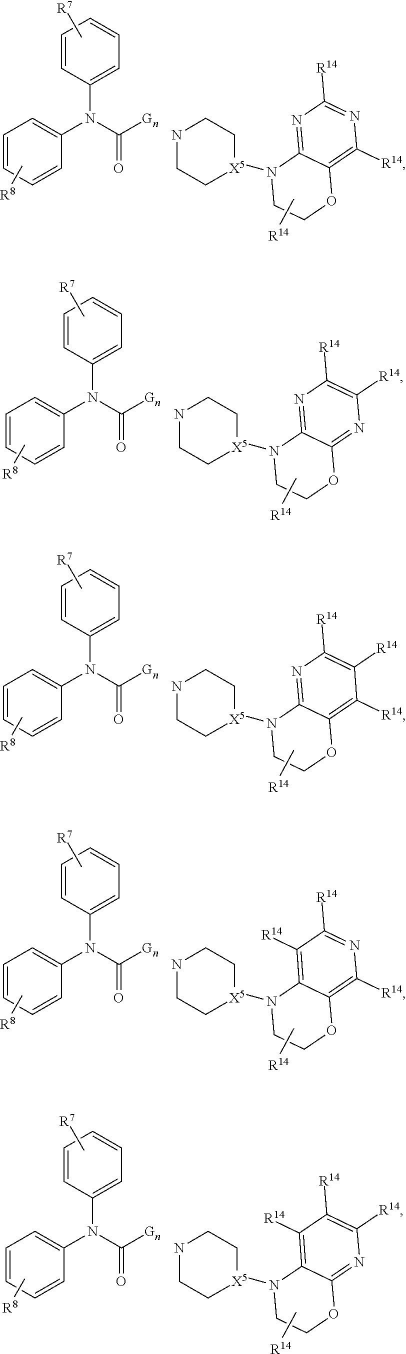 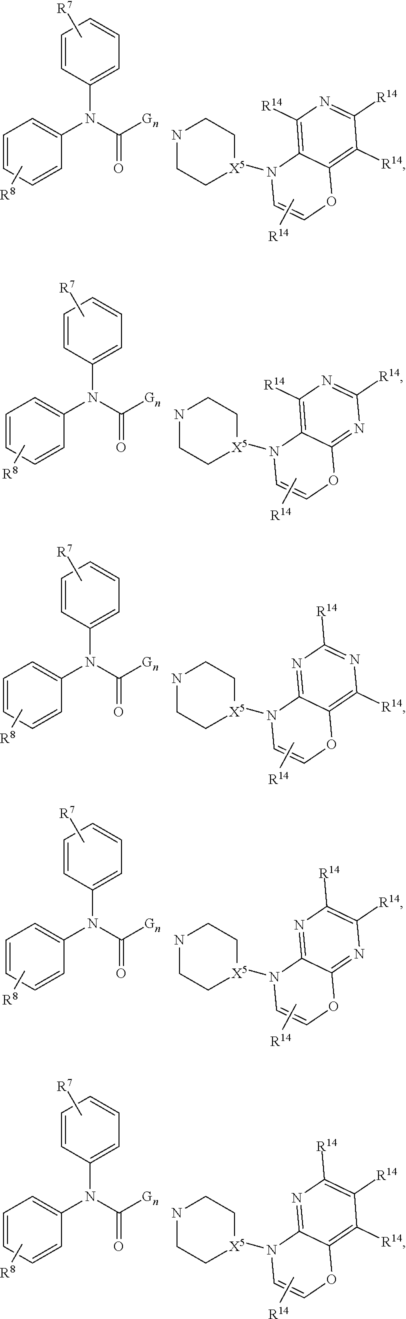 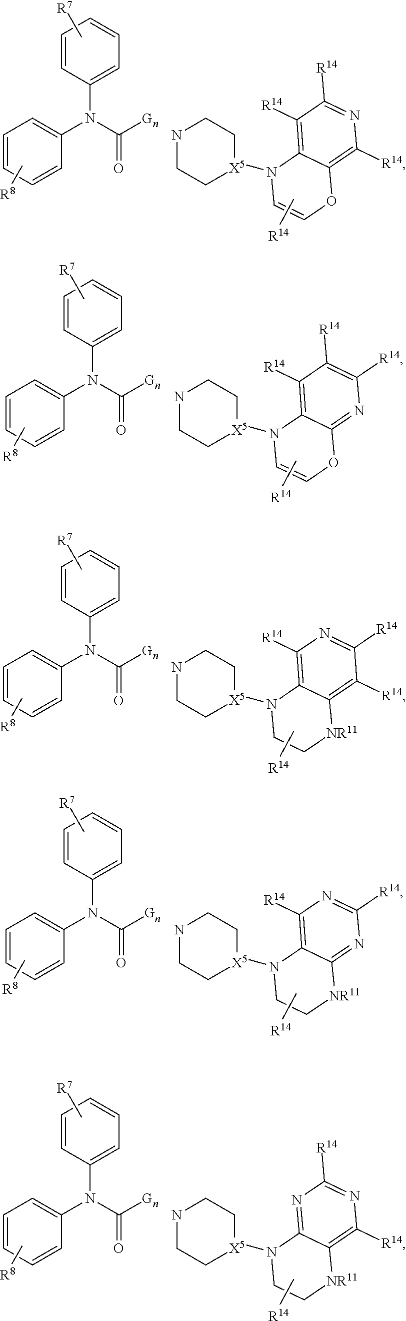 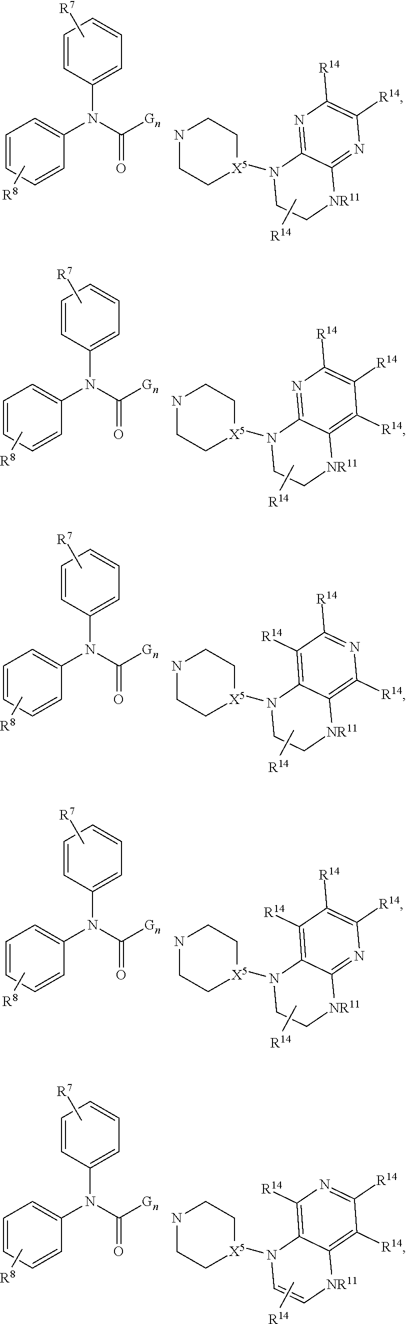 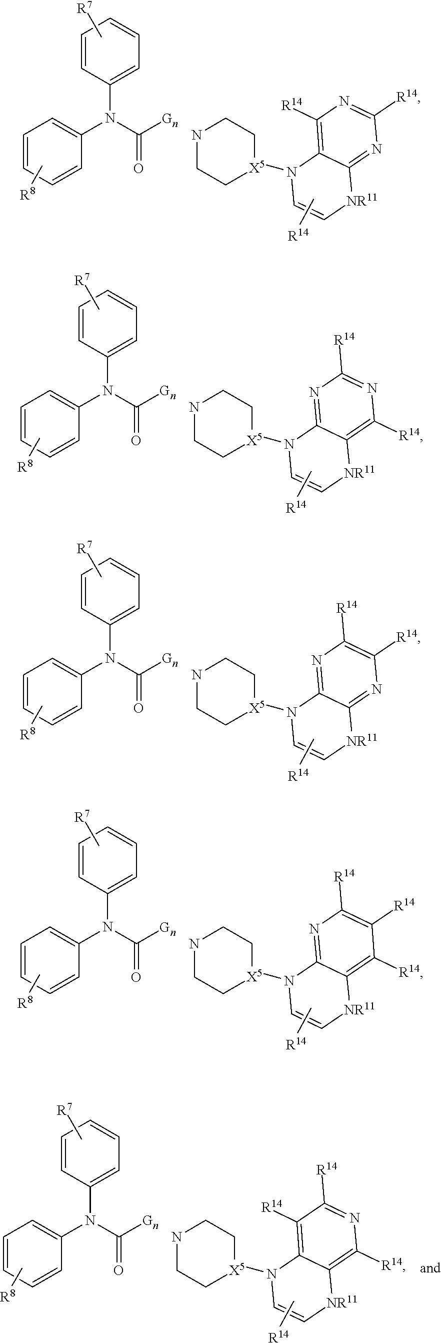 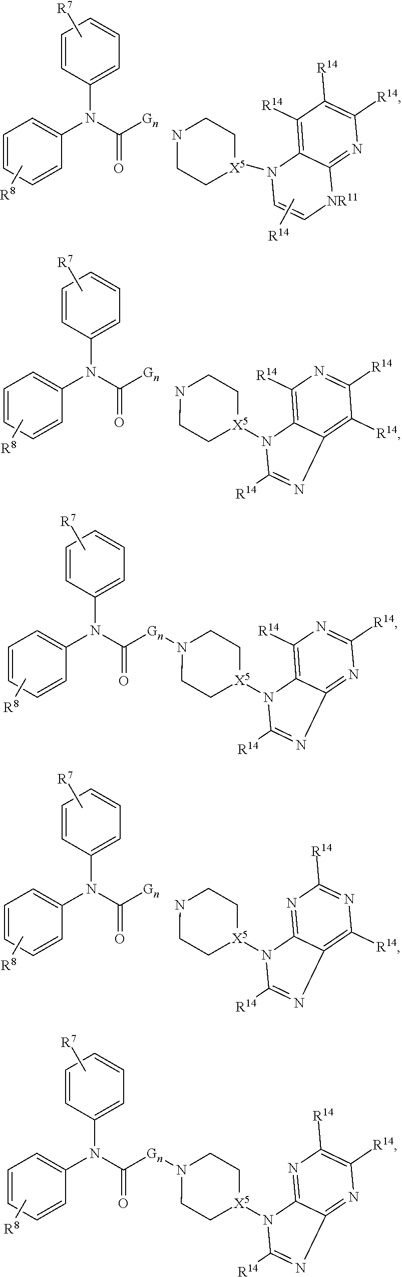 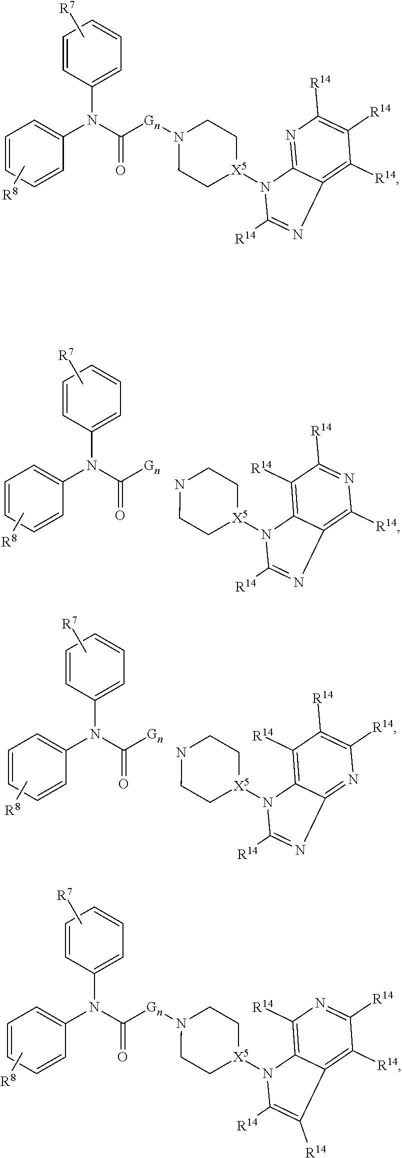 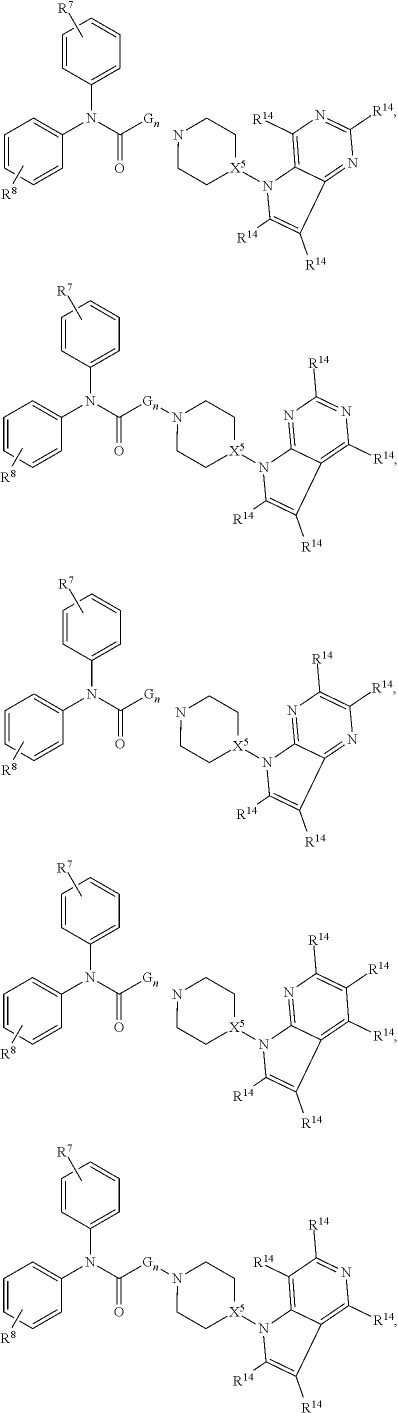 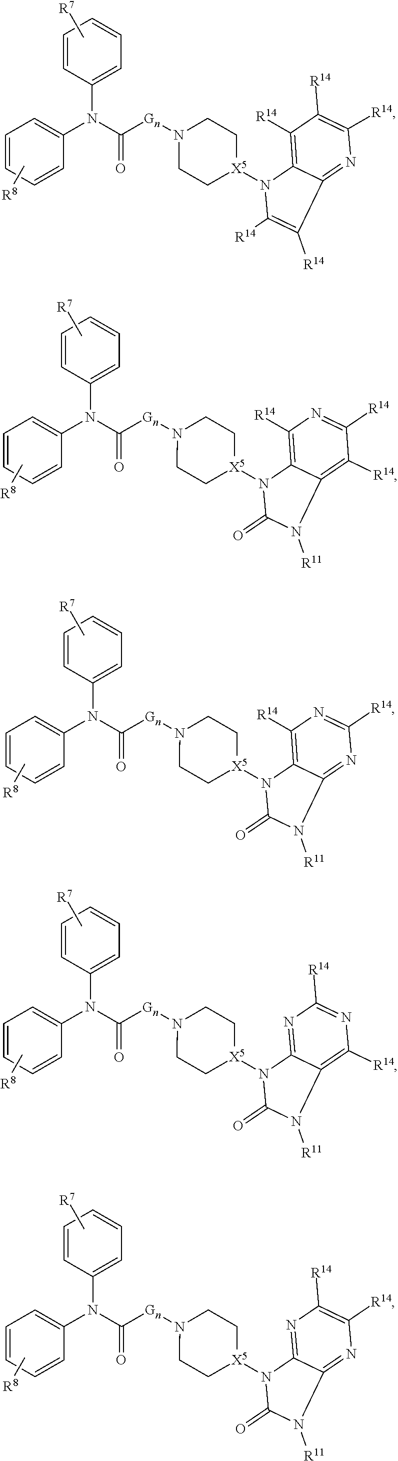 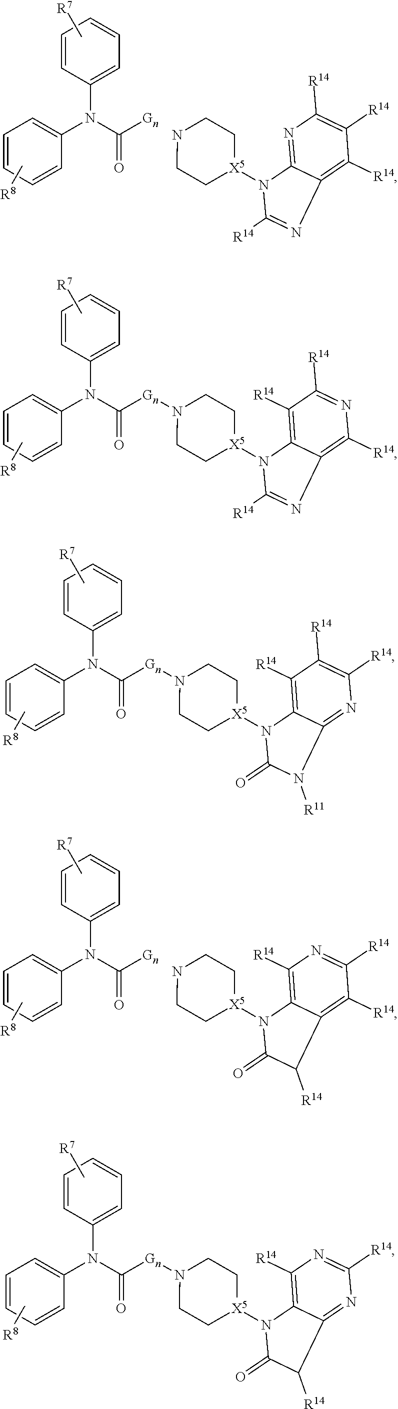 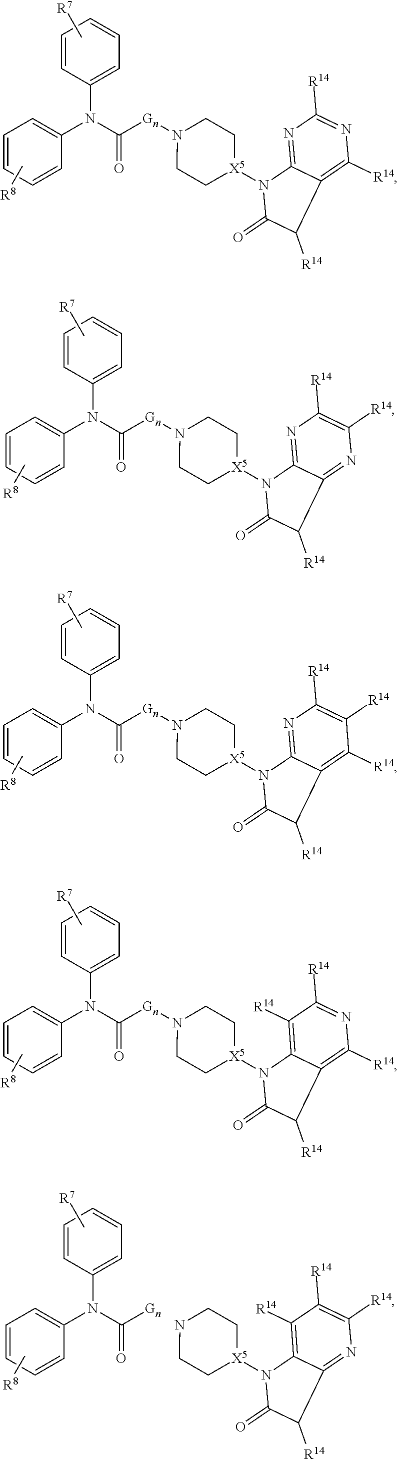 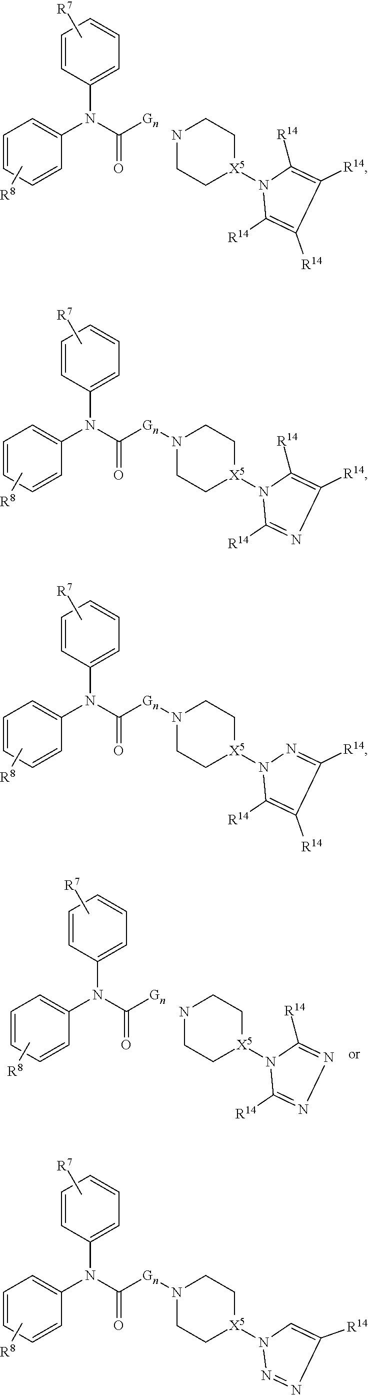
or a or a pharmaceutically acceptable salt, polymorph, prodrug, solvate or clathrate thereof.

59. A compound of the formula (V): 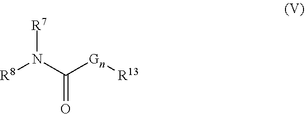
or a pharmaceutically acceptable salt, polymorph, prodrug, solvate or clathrate thereof,
wherein:
n is 0, 1 or 2;
each G is independently alkyl or C(O);
R⁷ and R⁸ are each independently halo, a carbon with at least one halo, alkyl, aryl, acyl or heterocyclyl; or
R⁷ and R⁸, together with the nitrogen atom to which they are attached, can form a heterocyclyl; and
R¹³ is a heterocyclyl group of the formula: 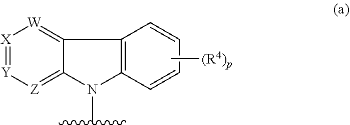
wherein W is N or C—R¹⁴; X is N or C—R¹⁴; Y is N or C—R¹⁴; and Z is N or C—R¹⁴; wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R⁴ is alkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and p is 0, 1, 2, 3 or 4; 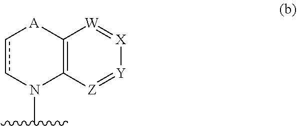
wherein A is S(O)ₓ, wherein x is 0, 1 or 2; O; C—R¹⁴, wherein R¹⁴ is hydrogen, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or A is N—R¹¹, wherein R¹¹ is a carbon with at least one halo, alkyl, aryl, acyl or heterocyclyl; 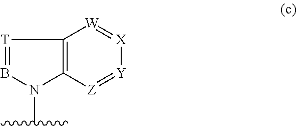
wherein T is CR¹⁴, wherein each R¹⁴ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or T is N; and
B is C—R¹⁴, wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; or B is N;

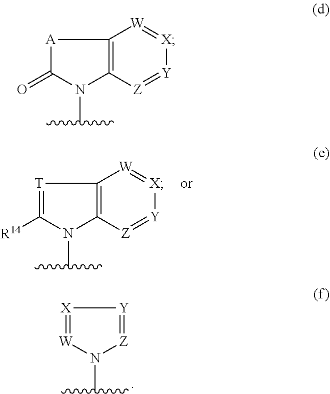

60. The compound of claim 59, wherein the compounds of the formula (V) are compounds of the formulae: 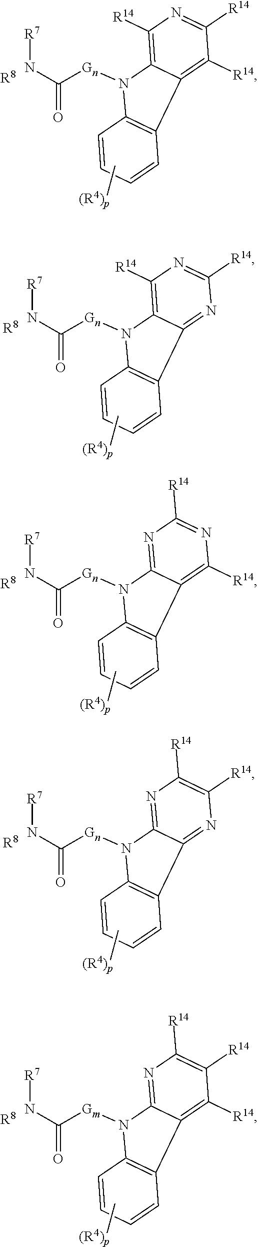 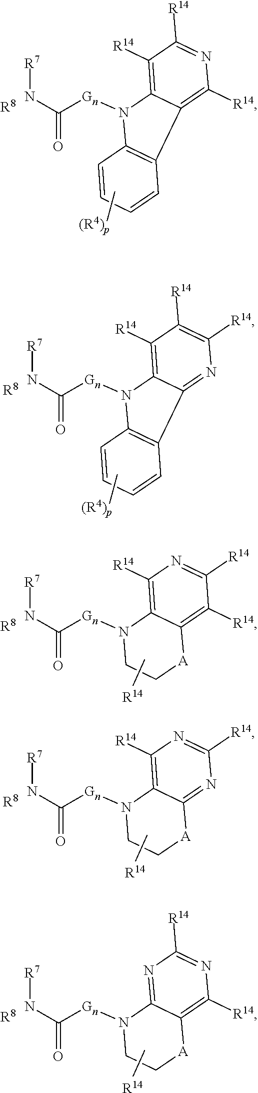 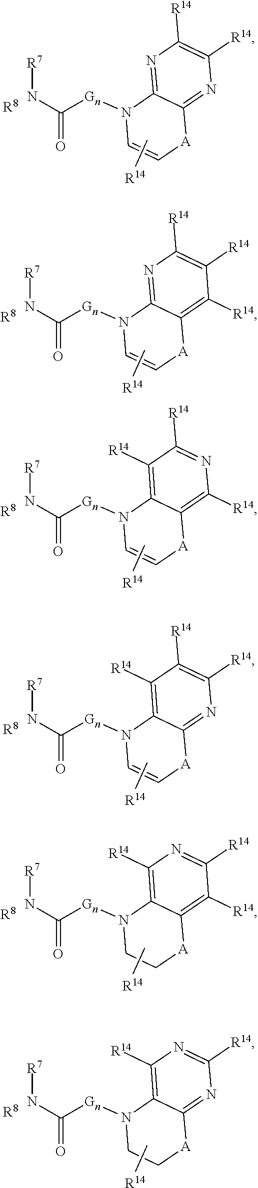 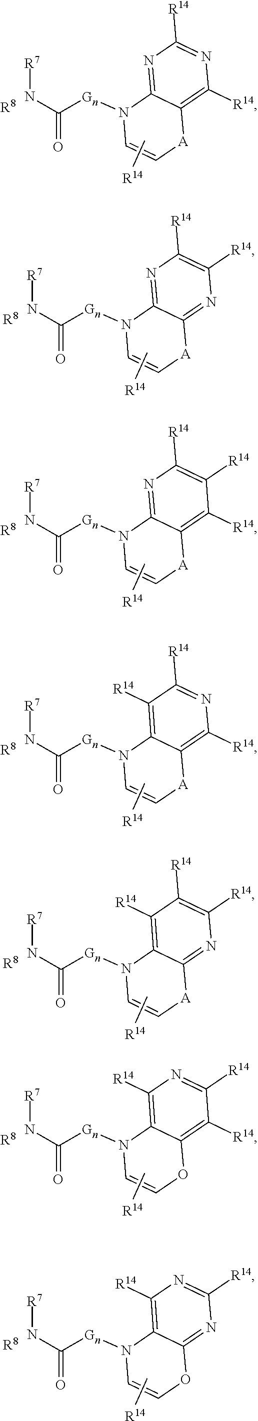 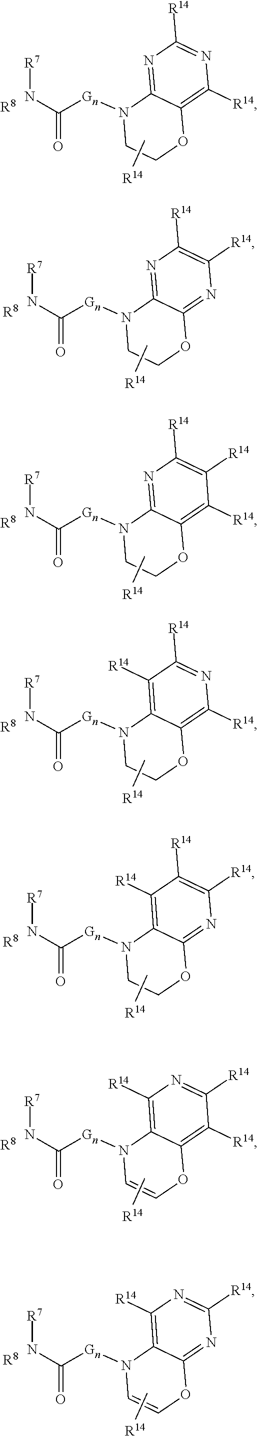 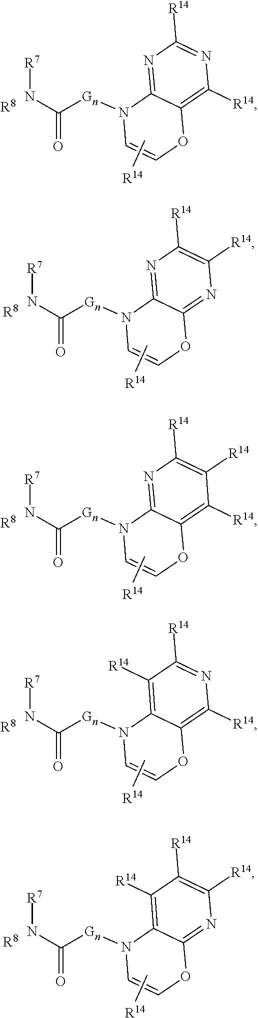 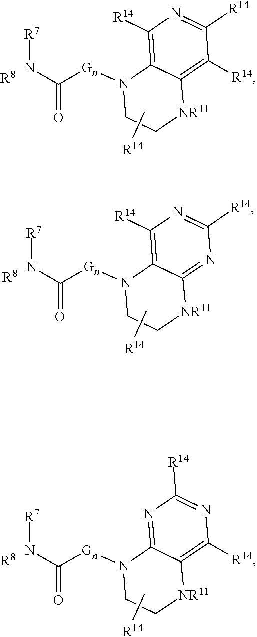 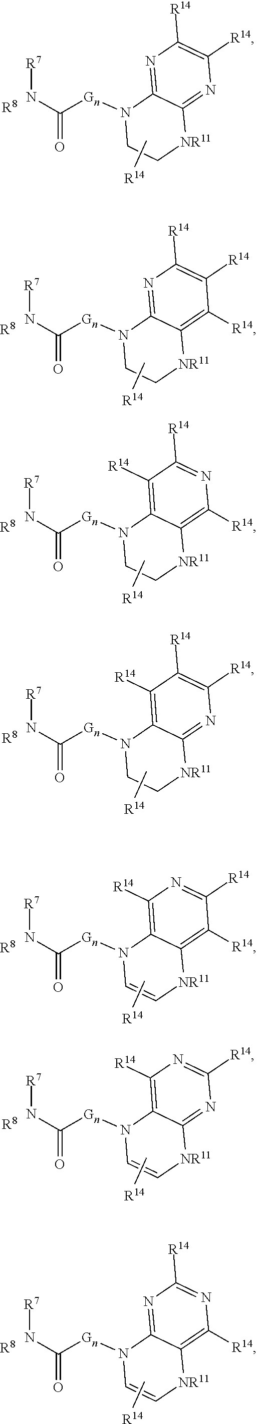 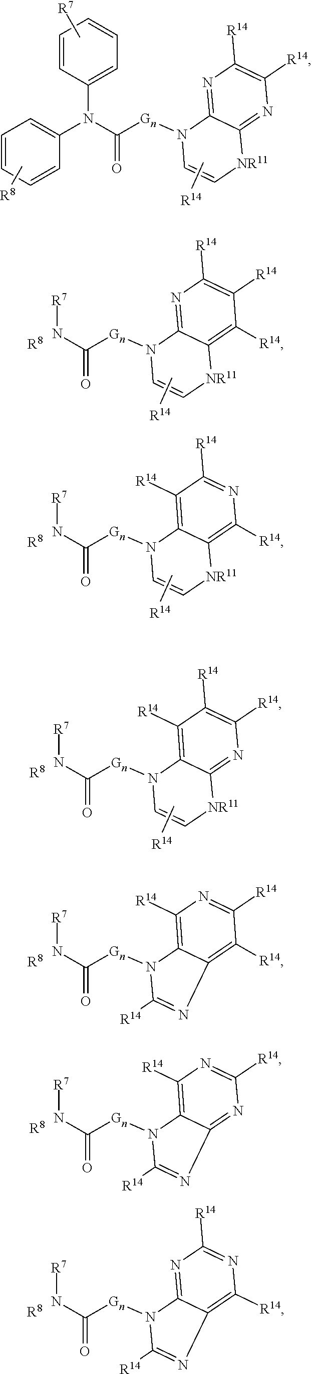 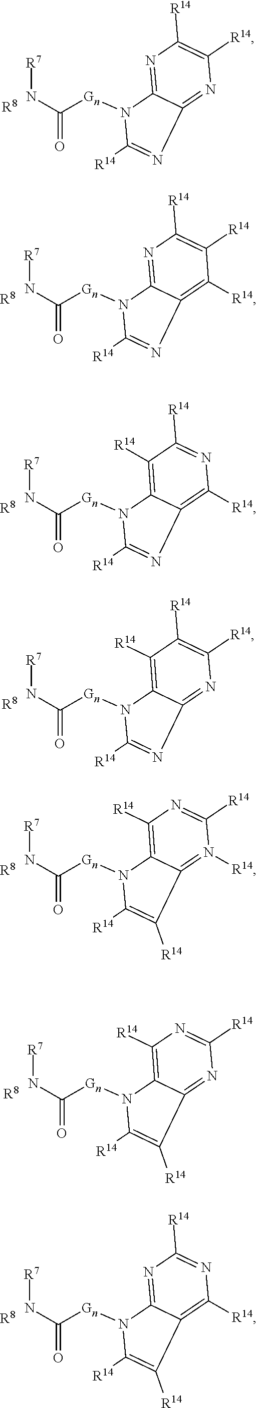 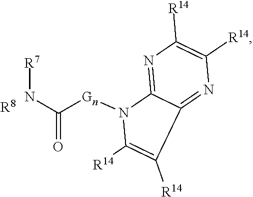 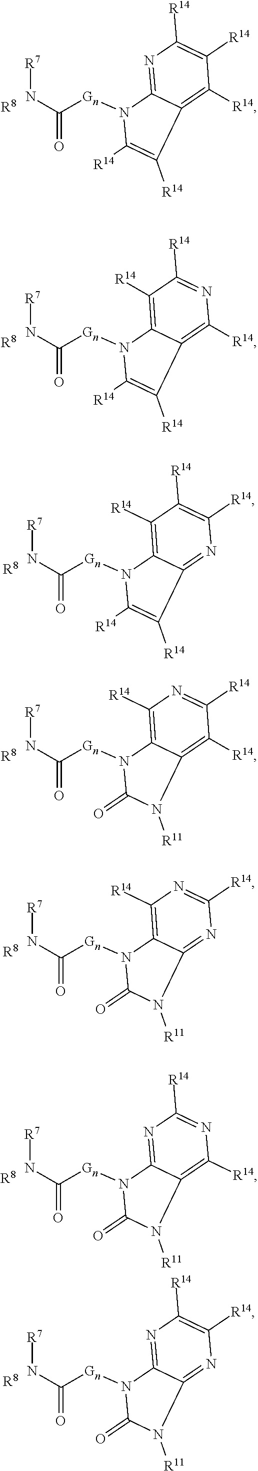 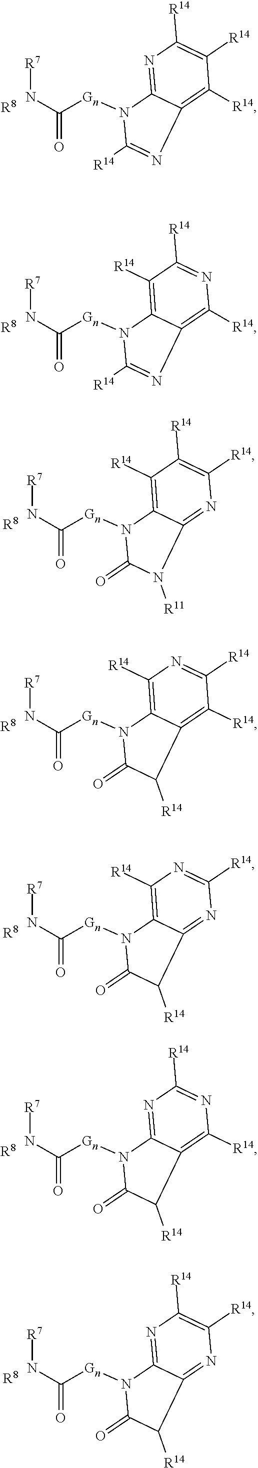 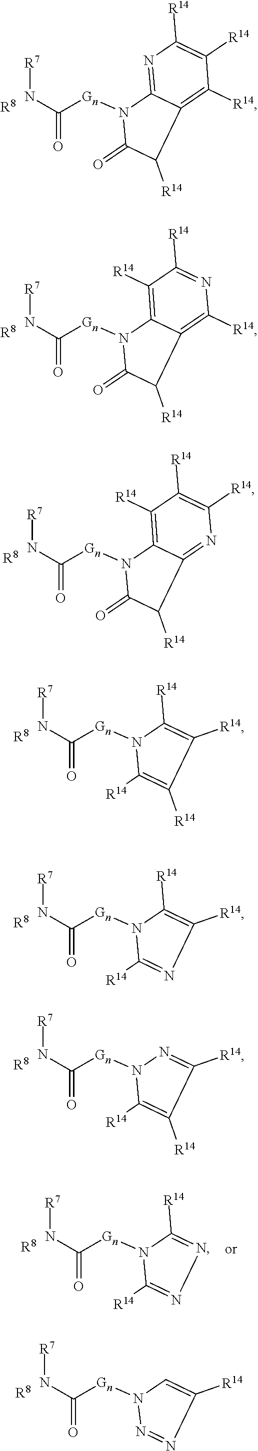
or a pharmaceutically acceptable salt, polymorph, prodrug, solvate or clathrate thereof.

60. A compound of the formula (VI): 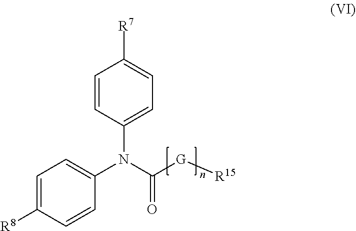
or a pharmaceutically acceptable salt, polymorph, prodrug, solvate or clathrate thereof,
wherein:
n is 0, 1 or 2;
each G is independently alkyl or C(O); and
R⁷ and R⁸ are each independently halo, a carbon with at least one halo, alkyl, aryl, acyl or heterocyclyl; and
R¹⁵ is a heterocyclyl group of the formula: 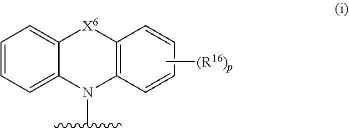
wherein X⁶ is alkyl, alkenyl, S, O or NR¹⁷; wherein R¹⁷ is hydrogen, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R¹⁶ is alkyl, haloalkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and
p is 0, 1, 2, 3 or 4; 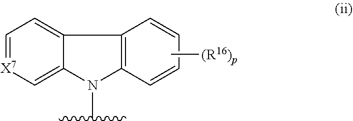
wherein X⁷ is N or C—R¹⁸; wherein R¹⁸ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and
R¹⁶ is alkyl, haloalkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and
p is 0, 1, 2, 3 or 4; 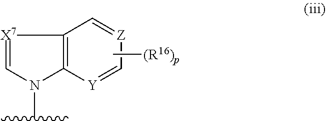
wherein X⁷ is N or C—R¹⁸; wherein R¹⁸ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R¹⁶ is alkyl, haloalkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and
p is 0, 1, 2, 3 or 4;
Y is N or C—R¹⁴; and Z is N or C—R¹⁴; wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; 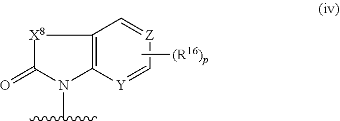
wherein X⁸ is NR¹⁹, wherein R¹⁹ is H, alkyl or aryl or X⁸ is C(R¹⁸)₂; wherein each R¹⁸ is independently hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;
R¹⁶ is alkyl, haloalkyl, cycloalkyl, aryl, heteroaryl, halo, amino, OR⁷, wherein R⁷ is hydrogen, alkyl, cycloalkyl, aryl or arylalkyl, S(O)ₓ, wherein x is 0, 1 or 2, acyl, amido or heterocyclyl; and
p is 0, 1, 2, 3 or 4;
Y is N or C—R¹⁴; and Z is N or C—R¹⁴; wherein R¹⁴ is hydrogen, an electron withdrawing group, alkyl, cycloalkyl, aryl, heteroaryl, acyl, amido or heterocyclyl, wherein each aryl or heteroaryl is optionally substituted with halo, amino, OR⁷, wherein R⁷ is hydrogen, alkyl, cycloalkyl, aryl or arylalkyl, S(O)ₓ, wherein x is 0, 1 or 2, acyl, amido or heterocyclyl;

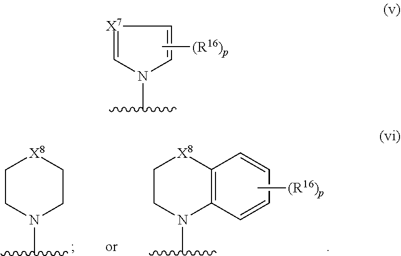

61. The compound of claim 60, wherein the compound is a compound of the formula: 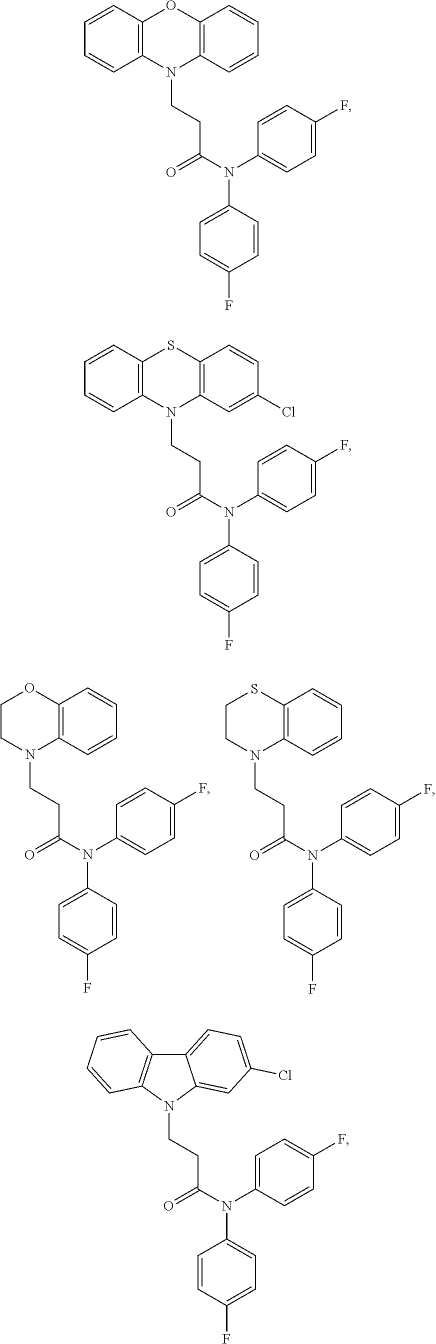 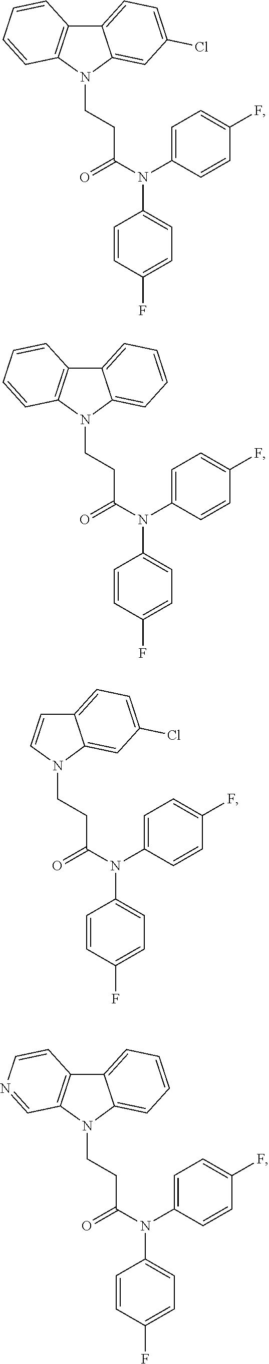 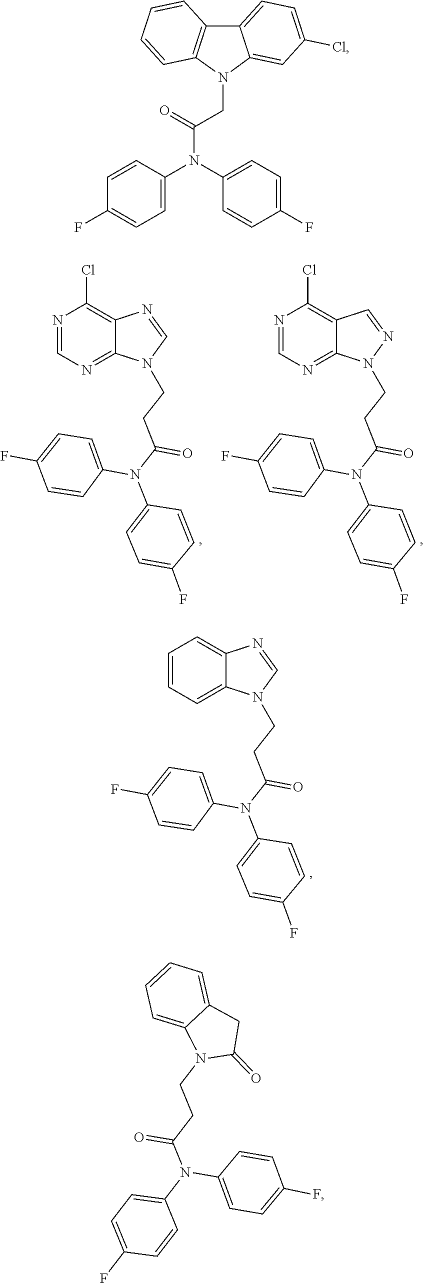 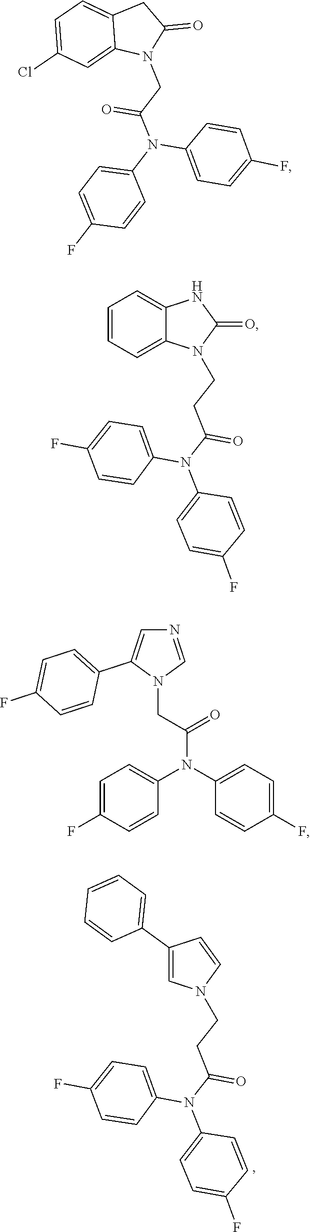 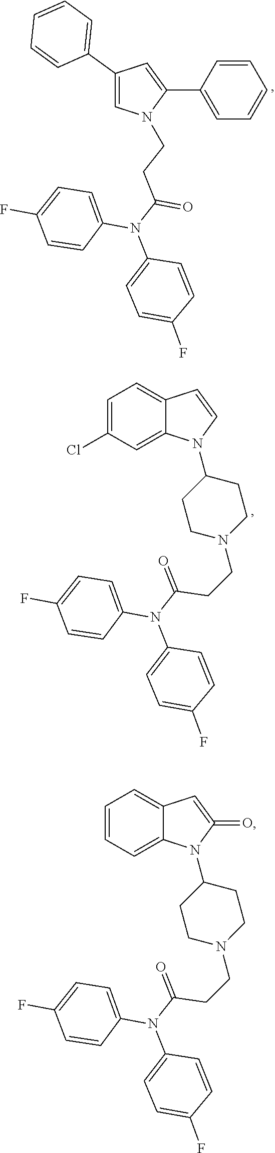 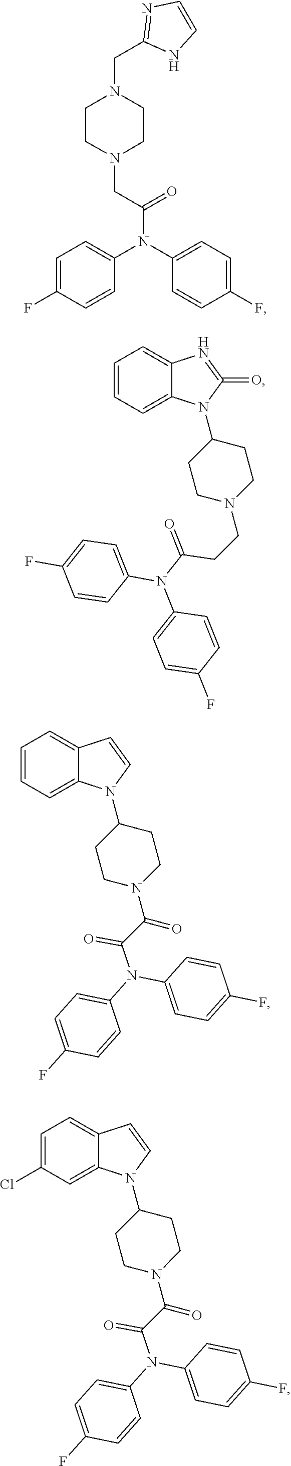 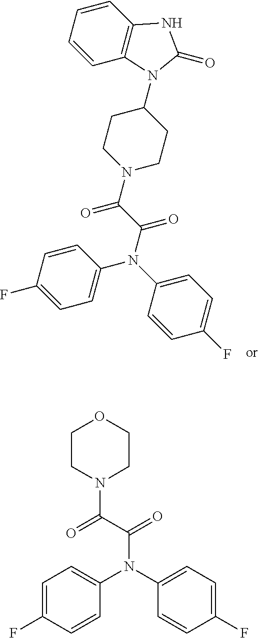 [SMILES: O=C(C(=O)N(c1ccc(F)cc1)c1ccc(F)cc1)N1CCC(n2c(=O)[nH]c3ccccc32)CC1 ; O=C(C(=O)N(c1ccc(F)cc1)c1ccc(F)cc1)N1CCOCC1]
or a pharmaceutically acceptable salt, polymorph, prodrug, solvate or clathrate thereof.

62. A pharmaceutical composition comprising one or more compounds of claim 51 and one or more pharmaceutically acceptable excipients.

63. A method for treating a neurodegenerative disease comprising administering a therapeutically effective amount of chlorpromazine or at least one compound of claim 51 to a subject in need thereof.

64. The method of claim 63, wherein the neurodegenerative disease is at least one of Parkinson's disease, Alzheimer's disease, Huntington's disease, and ALS.

65. A method for reducing, substantially eliminating or eliminating dysregulation of proteostasis comprising administering a therapeutically effective amount of chlorpromazine or at least one compound of claim 51 to a subject in need thereof.

66. A method for reducing, substantially eliminating or eliminating the accumulation of intrinsically disordered proteins comprising administering a therapeutically effective amount of chlorpromazine or at least one compound of claim 51 to a subject in need thereof.

67. A method for treating a neurodegenerative disease comprising administering at least one of a pharmaceutical composition comprising chlorpromazine or a pharmaceutical composition of claim 63 to a subject in need thereof.

68. A method for reducing, substantially eliminating or eliminating dysregulation of proteostasis comprising administering at least one of a pharmaceutical composition comprising chlorpromazine or a pharmaceutical composition of claim 62 to a subject in need thereof.

69. A method for reducing, substantially eliminating or eliminating the accumulation of intrinsically disordered proteins comprising administering at least one of a pharmaceutical composition comprising chlorpromazine or a pharmaceutical composition of claim 62 to a subject in need thereof.
